SEQUENCE LISTING (14 sequences)

SEQ ID NO: 1 - DNA, 20 bp, Synthetic construct
  source 1..20: mol_type = other DNA
ctaacactgaagagcgccgg

SEQ ID NO: 2 - DNA, 20 bp, Synthetic construct
  source 1..20: mol_type = other DNA
tgacaagagttccccgacac

SEQ ID NO: 3 - DNA, 20 bp, Synthetic construct
  source 1..20: mol_type = other DNA
ggccatcaaccggaaactca

SEQ ID NO: 4 - DNA, 20 bp, Synthetic construct
  source 1..20: mol_type = other DNA
tccatccaaggatgcggttg

SEQ ID NO: 5 - DNA, 20 bp, Synthetic construct
  source 1..20: mol_type = other DNA
gaagttgaacgagtaccgcc

SEQ ID NO: 6 - DNA, 20 bp, Synthetic construct
  source 1..20: mol_type = other DNA
atttggatttcaatgtgagg

SEQ ID NO: 7 - DNA, 20 bp, Synthetic construct
  source 1..20: mol_type = other DNA
agtatactcacgccacccac

SEQ ID NO: 8 - DNA, 20 bp, Synthetic construct
  source 1..20: mol_type = other DNA
acgtgtaaggcgaacgcctt

SEQ ID NO: 9 - DNA, 20 bp, Synthetic construct
  source 1..20: mol_type = other DNA
ggccaaccgtgaaaagatga

SEQ ID NO: 10 - DNA, 20 bp, Synthetic construct
  source 1..20: mol_type = other DNA
tacgaccagaggcatacagg

SEQ ID NO: 11 - DNA, 20 bp, Synthetic construct
  source 1..20: mol_type = other DNA
ccctgtgagcctatggactc

SEQ ID NO: 12 - DNA, 20 bp, Synthetic construct
  source 1..20: mol_type = other DNA
tgtggaagggaagacagagc

SEQ ID NO: 13 - DNA, 20 bp, Synthetic construct
  source 1..20: mol_type = other DNA
acatccagagccctcagttc

SEQ ID NO: 14 - DNA, 20 bp, Synthetic construct
  source 1..20: mol_type = other DNA
ggctccacagttcttcacac

(19) United States
(12) Patent Application Publication
(10) Pub. No.: US 2025/0144107 A1
(43) Pub. Date: May 8, 2025

(54) USE OF A PIKFYVE INHIBITOR IN COMBINATION WITH IMMUNOTHERAPY

(71) Applicant: The Regents of the University of Michigan, Ann Arbor, MI (US)

(72) Inventors: Arul M. Chinnaiyan, Ann Arbor, MI (US); Yi Bao, Ann Arbor, MI (US); Yuanyuan Qiao, Ann Arbor, MI (US)

(21) Appl. No.: 18/883,386

(22) Filed: Sep. 12, 2024

Related U.S. Application Data
(60) Provisional application No. 63/557,154, filed on Feb. 23, 2024.
(60) Provisional application No. 63/537,996, filed on Sep. 12, 2023.

(51) Int. Cl. A61K 31/5377; A61K 31/506; A61K 39/00; A61K 39/395; A61K 40/11; A61K 40/31; A61K 40/32; A61K 40/42; A61P 35/00; A61P 37/04

(52) U.S. Cl. CPC A61K 31/5377; A61K 31/506; A61K 39/0011; A61K 39/3955; A61K 40/11; A61K 40/31; A61K 40/32; A61K 40/421; A61P 35/00; A61P 37/04

(57) ABSTRACT

The present disclosure provides methods for treating cancer comprising administering 13-isobutyl-4-methyl-10-(pyrimidin-2-ylamino)-1,2,4,7,8,13-hexahydro-6H-indazolo[5,4-a]pyrrolo[3,4-c]carbazol-6-one or N-[(E)-(3-methylphenyl)methylideneamino]-6-morpholin-4-yl-2-(2-pyridin-2-ylethoxy)pyrimidin-4-amine to a subject in combination with adoptive cell therapy (ACT) or a personalized cancer vaccine to a subject.

### CROSS-REFERENCE TO RELATED APPLICATIONS

The present application claims priority to U.S. Provisional Application No. 63/537,996, filed Sep. 12, 2023, and U.S. Provisional Application No. 63/557,154 filed Feb. 23, 2024, which are incorporated herein by reference in their entireties.

### STATEMENT OF GOVERNMENTAL INTEREST

This invention was made with government support under CA231996 and CA186786 awarded by the National Institutes of Health. The government has certain rights in the invention.

### SEQUENCE LISTING

The text of the computer readable sequence listing filed herewith, titled “UM_42362_203_SequenceListing.xml”, created Sep. 12, 2024, having a file size of 13,371 bytes, is hereby incorporated by reference in its entirety.

### BACKGROUND

### Field

The present disclosure provides methods for treating cancer comprising administering a PIKfyve inhibitor, e.g., 13-isobutyl-4-methyl-10-(pyrimidin-2-ylamino)-1,2,4,7,8,13-hexahydro-6H-indazolo[5,4-a]pyrrolo[3,4-c]carbazol-6-one (ESK981) or N-[(E)-(3-methylphenyl)methylideneamino]-6-morpholin-4-yl-2-(2-pyridin-2-ylethoxy)pyrimidin-4-amine (apilimod), to a subject in combination with an immunotherapy, e.g., adoptive cell therapy (ACT) or a personalized cancer vaccine.

### Background

Immunotherapy harnesses the power of the immune system to fight cancer. See, e.g., Sharma, P. et al., Cell 2015; 161(2):205-14; Sharma, P., et al., Cancer Discov, 2021; 11(4):838-857; Rosenberg, S. A. et al., Science 2015; 348(6230):62-68; Saxena, M., et al., Nat. Rev. Cancer 2021; 21(6):360-378. Many patients, however, fail to benefit from immunotherapies by exhibiting primary resistance. See, e.g., Janjigian, Y. Y., et al., Lancet 2021; 398(10294):27-40; Sahin, U., et al., Nature 2020; 585(7823):107-112; Larkin, J. et al., N. Engl. J. Med. 2015; 373(1):23-34; Borghaei, H. et al., N. Engl. J. Med. 2015; 373(17):1627-39. Understanding what enables successful immunotherapy treatment and developing strategies that overcome resistance to immunotherapies remains a critical pursuit. See, e.g., Janjigian, Y. Y., et al., Lancet 2021; 398(10294):27-40; Sahin, U., et al., Nature 2020; 585(7823):107-112; Larkin, J. et al., N. Engl. J. Med. 2015; 373(1):23-34; Borghaei, H. et al., N. Engl. J. Med. 2015; 373(17):1627-39.

The role of CD8+ T cells in anticancer immunity and the clinical success of immunotherapies has been well-established. See, e.g., Matsushita, H. et al., Nature 2012; 482(7385):400-404; Robbins, P. F. et al., Nat. Med. 2013; 19(6):747-52; Gubin, M. M. et al., Nature 2014; 515(7528):577-581; Waldman, A. D. et al., Nat. Rev. Immunol. 2020; 20(11):651-668. For example, antigen presentation by major histocompatibility complex class I (MHC-I) on tumor cells is crucial for CD8+ T cells to recognize malignant cells. See, e.g., Dhatchinamoorthy, K. et al., Frontiers in Immunol. 2021; 12; Jhunjhunwala, S. et al., Nat. Rev. Cancer 2021; 21(5):298-312. While complete genetic loss of MHC-I components is rare, malignant cells often down-regulate expression of MHC-I, leading to escape from CD8+ T cell recognition and failure of immunotherapies. See, e.g., Dhatchinamoorthy, K. et al., Frontiers in Immunol. 2021; 12; Jhunjhunwala, S., et al., Nat. Rev. Cancer 2021; 21(5):298-312; Ennishi, D. et al., Cancer Discov, 2019; 9(4):546-563; Yamamoto, K. et al., Nature 2020; 581(7806):100-105; Lee, J. H., et al., Nat. Commun. 2020; 11(1):1897.

Autophagy has recently been found to degrade MHC-I, resulting in decreased MHC-I surface expression in pancreatic ductal adenocarcinoma (PDAC) and several non-small-cell lung cancer cell lines. See, e.g., Yamamoto, K. et al., Nature 2020; 581(7806):100-105. PIKfyve plays an essential role in autophagy and lysosomal adaptation processes, whereby inhibiting PIKfyve impairs autophagy via blocking autophagic flux and exhibits antitumor efficacy. See, e.g., Qiao, Y. et al., Nat. Cancer 2021; 2:978-993; Gayle, S., et al., Blood 2017; 129(13):1768-1778; Sharma, G. et al., Autophagy 2019; 15(10):1694-1718; Gayle, S. et al., Autophagy 2017; 13(6):1082-1083. Inhibition of autophagy restores tumor-specific MHC-I surface expression, leading to increased intratumoral CD8+ T cells and immune checkpoint blockade (ICB) sensitization in mouse PDAC models. See, e.g., Yamamoto, K. et al., Nature 2020; 581(7806):100-105. Concordantly, accumulating evidence suggests that autophagy inhibition may augment antitumor immunity and enhance the efficacy of ICB therapy. See, e.g., Wei, H. et al., Genes Dev. 2011; 25(14):1510-1527; Noman, M. Z. et al., Sci. Adv. 2020; 6(18):7881; Mgrditchian, T. et al., Proc. Natl. Acad. Sci. USA 2017; 114(44):E9271-E9279. However, autophagy inhibitors tested in the clinic, chloroquine and hydroxychloroquine, show limited cellular uptake in acidic conditions. See, e.g., Pellegrini, P. et al., Autophagy 2014; 10(4):562-571. This is potentially attributed to the low clinical efficacy of these autophagy inhibitors, as the tumor microenvironment is acidic. See, e.g., Karasic, T. B. et al., Jama Oncology 2019; 5(7):993-998; Zeh, H. J. et al., Clinical Cancer Research 2020; 26(13):3126-3134; Arnaout, A. et al., Breast Cancer Res. and Treat. 2019; 178(2):327-335; Boedtkjer, E. et al., Ann. Rev. Phy. 2020; 82:103-126. Furthermore, chloroquine and hydroxychloroquine are non-specific autophagy inhibitors. See, e.g., Mauthe, M. et al., Autophagy 2018; 14(8):1435-1455; Schrezenmeier, E. et al., Nat. Rev. Rheuma. 2020; 16(3):155-166. Novel therapeutic strategies are needed to target autophagy.

PIKfyve is a class III lipid kinase that phosphorylates PI3P (phosphatidylinositol 3-phosphate) to generate PI(3,5)P2 (phosphatidylinositol 3,5-biphosphate). PIKfyve, which is related to the more well-known lipid-related kinase PIK3CA, plays an essential role in autophagy and lysosomal adaptation processes. PIKfyve inhibition has been shown to mediate autophagy dysfunction by blocking autophagic flux and to exhibit antitumor efficacy in various cancer types. See, e.g., Qiao, Y., et al., Nat. Cancer 2021; 2:978-993; Gayle, S., et al., Blood 2017; 129(13):1768-1778; Sharma, G. et al., Autophagy 2019; 15(10):1694-1718; Gayle, S. et al., Autophagy 2017; 13(6):1082-1083. At least two PIKfyve inhibitors, apilimod, and ESK981 (formerly CEP-11981), are in clinical development for cancer therapy See, e.g., Harb, W. A. et al., Blood 2017; 130; Diefenbach, C. S. M. et al., J. Clin. Oncology 2020; 38 (15); Pili, R. et al., Invest. New. Drugs 2014; 32(6):1258-68.

### BRIEF SUMMARY

In one aspect, the present disclosure provides methods for treating cancer in a subject in need thereof, the method comprising administering a therapeutically effective amount of:
(a) 13-isobutyl-4-methyl-10-(pyrimidin-2-ylamino)-1,2,4,7,8,13-hexahydro-6H-indazolo[5,4-a]pyrrolo[3,4-c]carbazol-6-one (ESK981); or
(b) N-[(E)-(3-methylphenyl)methylideneamino]-6-morpholin-4-yl-2-(2-pyridin-2-ylethoxy)pyrimidin-4-amine (apilimod); or
(c) ESK981 and apilimod,
to the subject in combination with a therapeutically effective amount of an immunotherapy.

In another aspect, the present disclosure provides methods for treating cancer in a subject in need thereof, the method comprising administering a therapeutically effective amount of ESK981 and/or apilimod to the subject in combination with a therapeutically effective amount of an immunotherapy, wherein the cancer is characterized as overexpressing PIKfyve.

In another aspect, the immunotherapy is immune checkpoint blockade.

In another aspect, the immunotherapy is adoptive cell therapy.

In another aspect, the immunotherapy is a personalized cancer vaccine.

### BRIEF DESCRIPTION OF THE DRAWINGS

FIG. 1A is a series of 6 western blot images showing levels of PikfyveKO1 and PikfyveKO2 proteins in KPC1361 and B16-F10 cells.

FIG. 1B is a series of 6 images showing MHC-I localization in KPC1361 cells with non-targeting single-guide RNA (control), or independent single-guide RNAs depleting Pikfyve (Pikfyve KO1 and Pikfyve KO2).

FIG. 1C (Left) is line graphs of flow cytometry measuring MHC-I surface expression in control or Pikfyve KO cells, treated with or without IFN-γ; and (Right) bar graphs of quantification of flow cytometry measuring MHC-I surface expression in control or Pikfyve KO cells, treated with or without IFN-γ.

FIG. 1D is a series of 6 images showing MHC-I localization in IFN-γ stimulated KPC1361 cells treated with DMSO, Apilimod, or ESK981.

FIG. 1E (Left) is line graphs of flow cytometry measuring MHC-I surface expression in KPC1361 cells, stimulated with or without IFN-γ, treated with DMSO, Apilimod, or ESK981; (Right) are bar graphs of quantification of flow cytometry measuring MHC-I surface expression in KPC1361 cells, stimulated with or without IFN-γ, treated with DMSO, Apilimod, or ESK981.

FIG. 1F (Left) is line graphs of flow cytometry measuring MHC-I surface expression in KPC1361 cells with or without Pikfyve KO, treated with DMSO, Bafilomycin, chloroquine, Apilimod, or ESK981; (Right) is bar graphs of quantification of flow cytometry measuring MHC-I surface expression in KPC1361 cells with or without Pikfyve KO, treated with DMSO, Bafilomycin, chloroquine, Apilimod, or ESK981.

FIG. 1G (Left) is a line graph of flow cytometry measuring MHC-I surface expression in KPC1361 cells with non-targeting single-guide RNA (control), or single-guide RNAs depleting Atg5 or Atg7, treated with DMSO or Apilimod; (Right) is a bar graph of quantification of flow cytometry measuring MHC-I surface expression in KPC1361 cells with non-targeting single-guide RNA (control), or single-guide RNAs depleting Atg5 or Atg7, treated with DMSO or Apilimod.

FIG. 2A (Left) is line graphs of flow cytometry measuring MHC-I surface expression in MyC-CaP cells, stimulated with or without IFN-γ, treated with DMSO, Apilimod, or ESK981; (Right) is a bar graph of quantification of flow cytometry measuring MHC-I surface expression in MyC-CaP cells, stimulated with or without IFN-γ, treated with DMSO, Apilimod, or ESK981.

FIG. 2B (Left) is line graphs of flow cytometry measuring MHC-I surface expression in MIA PaCa2 cells, stimulated with or without IFN-γ, treated with DMSO, Apilimod, or ESK981; (Right) is a bar graph of quantification of flow cytometry measuring MHC-I surface expression in MIA PaCa2 cells, stimulated with or without IFN-γ, treated with DMSO, Apilimod, or ESK981.

FIG. 2C (Left) is a line graph of flow cytometry measuring MHC-I surface expression in LNCaP cells, stimulated with or without IFN-γ, treated with DMSO, Apilimod, or ESK981; (Right) is a bar graph of quantification of flow cytometry measuring MHC-I surface expression in LNCaP cells, stimulated with or without IFN-γ, treated with DMSO, Apilimod, or ESK981.

FIG. 2D (Left) is a series of 3 images showing MHC-I expression in prostate cancer patient-derived xenografts (PDX) in severe combined immunodeficiency (SCID) mice treated with DMSO, Apilimod, or ESK981; (Right) is a bar graph showing Area of MHC-I positive expression in quantification of prostate cancer patient-derived xenografts (PDX) in severe combined immunodeficiency (SCID) mice treated with DMSO, Apilimod, or ESK981.

FIG. 2E is a series of 3 western blot images showing levels of MHC-I, LC3A/B-II, and H3 expression in KPC1361, MIA PaCa2, and LNCaP cells treated with DMSO, Apilimod, or ESK981.

FIG. 2F is a bar graph of RT-qPCR expression of H2-K1 and H2-D1 in KPC1361 cells treated with DMSO, Apilimod, or ESK981.

FIG. 2G is a series of 2 western blot images showing knockout efficacy on Atg5 or Atg7 in KPC1361 cells.

FIG. 3A is a western blot image showing ovalbumin (OVA) expression in KPC1361 and B16-F10 cells.

FIG. 3B is a line graph of flow cytometry showing surface expression of OVA-peptide (SIINFEKL) bound MHC-I (H-2Kb) in IFN-γ stimulated KPC1361 and B16-F10 cells.

FIG. 3C (Left) is line graphs of flow cytometry showing surface expression of an OVA-peptide (SIINFEKL) bound MHC-I (H-2Kb) in IFN-γ stimulated KPC1361 and B16-F10 cells, treated with DMSO, Apilimod, or ESK981; (Right) are bar graphs of quantification of flow cytometry measuring surface expression of an OVA-peptide (SIINFEKL) bound MHC-I (H-2Kb) in IFN-γ stimulated KPC1361 and B16-F10 cells, treated with DMSO, Apilimod, or ESK981.

FIG. 4A (Left) are bar graphs of KPC1361 and B16-F10 cell viability, with or without ovalbumin overexpression (OVA) treated with DMSO, Apilimod, or ESK981 then subsequent co-culture with activated CD8+ T cells; (Right) are bar graphs of control or Pikfyve-null KPC1361 and B16-F10 cell viability, with or without ovalbumin overexpression (OVA) treated with DMSO, Apilimod, or ESK981 then subsequent coculture with activated CD8+ T cells.

FIG. 4B (Left) is a series of 6 images of flow cytometry of the IFN-γ⁺ and Ki67⁺ cells in the activated CD8⁺ T cells co-cultured with the indicated cancer cells in FIG. 4A; (Right) are bar graphs of quantification of flow cytometry measuring the IFN-γ⁺ and Ki67⁺ cells in the activated CD8⁺ T cells cocultured with the indicated cancer cells in FIG. 4A.

FIG. 4C (Left) is line graphs of flow cytometry measuring surface expression of an OVA-peptide (SIINFEKL) bound MHC-I (H-2Kb) in the control or Pikfyve-null KPC1361 cells, treated with or without IFN-γ; (Right) are bar graphs of quantification of flow cytometry measuring surface expression of an OVA-peptide (SIINFEKL) bound MHC-I (H-2Kb) in the control or Pikfyve-null KPC1361 cells, treated with or without IFN-γ.

FIG. 5A (Left) is a line graph showing tumor volume of pancreatic tumors injected, orthotopically, with control or Pikfyve-null KPC1361 cells; (Right) is a box plot of tumor weight of pancreatic tumors, orthotropically, injected with control or Pikfyve-null KPC1361 cells.

FIG. 5B (Left) is a line graph showing tumor volume of pancreatic tumors injected, subcutaneously, with control or Pikfyve-null B16-F10 cells; (Right) is a box plot of tumor weight of pancreatic tumors injected, subcutaneously, with control or Pikfyve-null B16-F10 cells.

FIG. 5C (Left) is a series of 2 images of flow cytometry measuring the amount of total CD8⁺ T cells and proportion of activated or proliferative CD8⁺ T cells in the indicated tumors established as in FIG. 5A and FIG. 5B; (Right) are bar graphs of quantification of flow cytometry measuring the amount of total CD8⁺ T cells and proportion of activated or proliferative CD8⁺ T cells in the indicated tumors established as in FIG. 5A and FIG. 5B.

FIG. 5D (Left) is a line graph of flow cytometry measuring surface expression of fluorescently labeled MHC-I in KPC1361 cells in the indicated tumors established in FIG. 5A and FIG. 5B; (Right) is a bar graph quantification of flow cytometry measuring the surface expression of MHC-I in KPC1361 cells in the indicated tumors established in FIG. 5A and FIG. 5B.

FIG. 5E (Left) is a line graph of flow cytometry measuring surface expression of fluoresenctly labeled MHC-I in B16-F10 cells in the indicated tumors established in FIG. 5A and FIG. 5B; (Right) is a bar graph quantification of flow cytometry measuring the surface expression of MHC-I in B16-F10 cells in the indicated tumors established in FIG. 5A and FIG. 5B.

FIG. 5F (Left) is a box plot of tumor weight of tumors injected, subcutaneously, with control or Pikfyve-knockout B16-F10 cells to mice, with (αCD8) or without (IgG) CD8⁺ T cell depletion; (Right) is a line graph showing tumor volume of tumors injected, subcutaneously, with control or Pikfyve-knockout B16-F10 cells to mice, with (αCD8) or without (IgG) CD8⁺ T cell depletion.

FIG. 5G (Left) is a box plot of tumor weight of tumors injected, subcutaneously, with control, B2m or Pikfyve single knockout, or B2m and Pikfyve double knockout B16-F10 cells to mice; (Right) is a line graph showing tumor volume of tumors injected, subcutaneously, with control, B2m or Pikfyve single knockout, or B2m and Pikfyve double knockout B16-F10 cells to mice.

FIG. 6A is a series of 3 images of flow cytometry identifying CD8⁺ T cells.

FIG. 6B are bar graphs showing quantification of flow cytometry measuring activated or proliferative CD8⁺ T cells in the tumor-draining lymph nodes (TdLN) from KPC1361 pancreatic tumor bearing mice.

FIG. 6C is a series of 3 images of flow cytometry identifying GFP-labelled cancer cells in tumors.

FIG. 6D (Left) is a series of 3 images of MHC-I expression in mice with pancreatic tumors, from orthotopic injections of KPC1361 cells; (Right) is a bar graph of quantification of MHC-I expression in mice with pancreatic tumors, from orthotopic injections of KPC1361 cells.

FIG. 6E (Left) is a line graph of Hallmark pathways enriched by bulk RNA-sequencing of Pikfyve-KO tumors; (Right) is a line graph of the Hallmark pathway enriched by IFN-γ.

FIG. 6F are bar graphs of the quantification of CD8⁺ T cells in KPC1361 pancreatic tumors enriched by IFN-γ

FIG. 7A (Left) is a series of 2 images of flow cytometry identifying CD8⁺ T cells in the blood from tumor-bearing mice treated with isotype control (IgG) or anti-mouse CD8 antibody (αCD8); (Right) is a bar graph of quantification of CD8⁺ T cells in the blood from tumor-bearing mice treated with isotype control (IgG) or anti-mouse CD8 antibody (αCD8).

FIG. 7B (Left) is a box plot of tumor weight of tumors injected, orthotopically, with control, Pikfyve-null KPC1361 cells in mice, with or without depletion of CD8⁺ T cells as in FIG. 7A; (Right) is a line graph of tumor volume of tumors injected, orthotopically, with control, Pikfyve-null KPC1361 cells in mice, with or without depletion of CD8⁺ T cells as in FIG. 7A.

FIG. 7C is a series of 4 western blots showing Beta-2 microglobulin (B2M) expression after B2m depletion by single-guide RNA (B2m KO) in KPC1361 and B16-F10 cells.

FIG. 7D (Left) is a box plot of tumor weight of pancreatic tumors injected, orthotopically, with control, B2m or Pikfyve single knockout, or B2m and Pikfyve double knockout KPC1361 cells to mice; (Right) is a line graph of tumor volume of pancreatic tumors injected, orthotopically with control, B2m or Pikfyve single knockout, or B2m and Pikfyve double knockout KPC1361 cells to mice.

FIG. 8A (Left) is a box plot of tumor volume of the efficacy of immune checkpoint inhibitors in combination with apilimod or ESK981 treatment against metastasis in a subcutaneous B16BL6 tumor; (Middle) is a series of 12 images of the efficacy of immune checkpoint inhibitors in combination with apilimod or ESK981 treatment against metastasis in a subcutaneous B16BL6 tumor; (Right) is a box plot of the counts of lung tumor metastasis treated with immune checkpoint inhibitors in combination with apilimod or ESK981.

FIG. 8B is a line graph of the efficacy of α-PD1 in combination with apilimod or ESK981 against pancreatic tumors from orthotopically injected KPC1361 cells to mice.

FIG. 8C (Left) is a line graph of flow cytometry measuring surface expression of MHC-I on GPF-labelled cancer cells in pancreatic tumors treated with α-PD1 in combination with apilimod or ESK981; (Right) is a bar graph of quantification of flow cytometry measuring surface expression of MHC-I on GPF-labelled cancer cells in pancreatic tumors treated with α-PD1 in combination with apilimod or ESK981.

FIG. 8D (left) is a series of 3 images of flow cytometry measuring the amount of total CD8⁺ T cells, and proportion of activated or proliferative CD8⁺ T cells in pancreatic tumors treated with α-PD1 in combination with apilimod or ESK981; (Right) are bar graphs of flow cytometry quantification of the amount of total CD8⁺ T cells, and proportion of activated or proliferative CD8⁺ T cells in pancreatic tumors treated with α-PD1 in combination with apilimod or ESK981.

FIG. 8E (Left) is a bar graph of the patient's proportion with tumors showing high or low pretreatment PIKfyve mRNA levels in complete response (CR) group; (Right) is a line graph of the overall survival of patients with tumors showing high or low pretreatment PIKfyve mRNA levels treated with immune checkpoint inhibitors.

FIG. 8F is a line graph of the overall survival of patients with tumors showing high or low pretreatment PIKfyve mRNA levels treated with immune checkpoint inhibitors.

FIG. 9A is a line graph of the survival of pancreatic tumor bearing mice, orthotopically injected with KPC1361 cells and treated with an immune checkpoint inhibitor, apilimod, or ESK981 individually, or in combination with each other.

FIG. 9B is a line graph of the body weight of pancreatic tumor bearing mice, orthotopically injected with KPC1361 cells and treated with an immune checkpoint inhibitor, apilimod, or ESK981 individually, or in combination with each other.

FIG. 9C is a box plot of Cox regression analysis for the overall survival of mice with pre-treatment PIKfyve expression.

FIG. 10A is an experimental timeline of the efficacy against subcutaneous ovalbumin-expressing B16-F10 (B16-F10-OVA) tumors treated with adoptive cell therapy (ACT) in combination with apilimod or ESK981.

FIG. 10B is a series of 6 line graphs showing the growth of subcutaneous ovalbumin-expressing B16-F10 (B16-F10-OVA) tumors treated with adoptive cell therapy (ACT) in combination with apilimod or ESK981.

FIG. 10C is a line graph of the survival of mice with subcutaneous ovalbumin-expressing B16-F10 (B16-F10-OVA) tumors treated with adoptive cell therapy (ACT) in combination with apilimod or ESK981.

FIG. 10D is an experimental timeline of the efficacy against subcutaneous B16-F10 tumors treated with a personalized therapeutic vaccine derived from irradiated B16-F10 cells and poly(I:C) in combination with apilimod or ESK981.

FIG. 10E is a series of 6 line graphs showing the growth of subcutaneous B16-F10 tumors treated with personalized therapeutic vaccine derived from irradiated B16-F10 cells and poly(I:C) in combination with apilimod or ESK981.

FIG. 10F is a line graph of the survival of mice with subcutaneous B16-F10 tumors treated with a personalized therapeutic vaccine derived from irradiated B16-F10 cells and poly(I:C) in combination with apilimod or ESK981.

FIG. 10G (Left) is a series of 2 images of flow cytometry identifying TRP2-tetramer⁺ CD8⁺ T cells; (Right) is a bar graph of flow cytometry quantification of TRP2-tetramer⁺ CD8⁺ T cells in tumors in subcutaneous B16-F10 tumors treated with personalized therapeutic vaccine derived from irradiated B16-F10 cells and poly(I:C) in combination with apilimod or ESK981.

FIG. 11A is a line graph of mice body weight treated with adoptive cell therapy (ACT) in combination with apilimod or ESK981.

FIG. 11B is a line graph of mice body weight treated with personalized vaccines in combination with apilimod or ESK981

FIG. 12A is a pie chart of the RECIST-defined response to treatment of patients with immune checkpoint blockade (ICB).

FIG. 12B is a line graph of the overall survival of patients treated with ICB by RECIST-defined treatment response.

FIG. 12C is a box plot of having complete response (CR) vs. not CR for 25 common Phase I/Phase II/FDA-approved drug target genes.

FIG. 12D is a box plot of the hazard ratios for progression-free survival of patients treated with ICB, by high vs. low gene expression for candidate drug target genes.

FIG. 12E is a box plot of the hazard ratios for overall survival of patients treated with ICB, by high vs. low gene expression for PIKfyve score and CD8⁺ T cell activation score.

FIG. 12F is a scatter plot of immune cells in pre-treatment tumors from patients with melanoma from scRNA-seq data.

FIG. 12G is a series of twelve box and whisker plots of PIKfyve expression across immune cell types in nonresponders “NR”) or responders “R” to ICB treatment.

FIG. 13A is a box and whisker plot of relative median fluorescent intensity of surface XCR1 in CD45⁺ cells of splenic DCs in PIKfyve inhibitor-treated non-tumor-bearing mice.

FIG. 13B is a box and whisker plot of cDC1 cells from spleens of mice treated with vehicle or apilimod, 30 mg/kg daily, after 5 days of treatment.

FIG. 13C (Left) is a line graph of relative median fluorescent intensity of the surface XCR1 on Pikfyve^{f/f} (“WT”) or Itgax^{Tg/0} Pikfyve^{f/f} (“KO”) cDCs after 9 days of culture. FIG. 13C (Right) is a box and whisker plot of the surface XCR1 on Pikfyve^{f/f} (“WT”) or Itgax^{Tg/0} Pikfyve^{f/f} (“KO”) cDCs after 9 days of culture.

FIG. 13D a western blot showing the total histone h3, loading control, and total XCR1 in Pikfyve WT vs. KO cDC lysates after 9 days of culture.

FIG. 13E (Left) is a line graph of relative median fluorescent intensity of the surface MHC-I (H-2kb, H-2kd) on WT or KO cDCs after 9 days of culture. FIG. 13E (Right) is a box and whisker plot of the surface MHC-I (H-2kb, H-2kd) on WT or KO cDCs after 9 days of culture.

FIG. 13F a western blot showing the total vinculin, loading control, and total MHC-I in PIKfyve WT vs. KO cDC lysates after 9 days of culture.

FIG. 13G (Left) is a line graph of the relative median fluorescent intensity of the surface MHC-I on DMSO or apilimod-treated cDCs treated for 20 hours after 6 days of culture. FIG. 13G (Right) is a box and whisker plot of the surface MHC-I on DMSO or apilimod-treated cDCs treated for 20 hours after 6 days of culture

FIG. 13H a western blot showing the total vinculin, loading control, and total MHC-I in DMSO or apilimod-treated, 20 hours, cDC lysates after 6 days of culture.

FIG. 13I (Left) is a line graph of the relative median fluorescent intensity of the surface MHC-II (MHC-IA-IE) on PIKfyve WT or KO cDCs after 9 days of culture. FIG. 13I (Right) is a box and whisker plot of the surface MHC-II (MHC-IA-IE) on PIKfyve WT or KO cDCs after 9 days of culture.

FIG. 13J a western blot showing the total vinculin, loading control, and total MHC-II (MHC-IA-IE) in PIKfyve WT vs. KO cDC lysates after 9 days of culture

FIG. 13K (Left) is a line graph of the relative median fluorescent intensity of the surface MHC-II on DMSO or apilimod-treated, 20 hours, cDCs treated after 6 days of culture. FIG. 13K (Right) is a box and whisker plot of the surface MHC-II on DMSO or apilimod-treated, 20 hours, cDCs treated after 6 days of culture.

FIG. 13L a western blot showing the total vinculin, loading control, and total MHC-II in DMSO or apilimod-treated, 20 hours, cDC lysates after 6 days of culture.

FIG. 13M a western blot showing the total histone H3, loading control, and total CD80 and CD86 in DMSO-treated, 20 hours, PIKfyve WT vs. KO cDC lysates after 9 days of culture.

FIG. 13N a western blot showing the total histone H3, loading control, and total CD80 and CD86 in apilimod-treated, 20 hours, PIKfyve WT vs. KO cDC lysates after 6 days of culture.

FIG. 13O is a series of two scatter plots of surface CD80 and CD86 on Pikfyve WT vs. KO cDC lysates after 9 days of culture.

FIG. 13P is a series of six scatter plots of surface CD80 and CD86 on DMSO or apilimod-treated cDCs, with or without 20 hours of PolyI:C, 50 μg/ml, or lipopolysaccharide (LPS), 50 ng/ml, treatment after 6 days of culture.

FIG. 13Q a western blot showing the total vinculin, loading control, and total CD40 in PIKfyve WT vs. KO cDC lysates after 9 days of culture.

FIG. 13R a western blot showing the total vinculin, loading control, and total CD40 in DMSO or apilimod-treated, 20 hours, cDC lysates treated with or without additional LPS, 50 ng/ml, after 6 days of culture day 6.

FIG. 13S is a box and whisker plot of the percentage of IFNγ⁺ OT-I cells after 72 hours of co-culture with PIKfyve WT vs. KO.

FIG. 13T is a box and whisker plot of the percentage of IFNγ⁺ OT-I cells after 72 hours of co-culture with DMSO or apilimod pre-treated cDCs with or without sOVA, 10 μg/ml.

FIG. 13U is a box and whisker plot of the percentage of IFNγ⁺ OT-II cells after 72 hours of co-culture with PIKfyve WT vs. KO.

FIG. 13V is a box and whisker plot of the percentage of IFNγ⁺ OT-II cells after 72 hours of co-culture with DMSO or apilimod pre-treated cDCs with or without sOVA, 10 μg/ml.

FIG. 14A is a line graph of the enrichment of NF-κB target genes in PIKfyve KO vs. WT cells after 9 days of culture.

FIG. 14B is a line graph of the enrichment of NF-κB target genes in DMSO or apilimod-treated, 3 hours, cDCs after 6 days of culture.

FIG. 14C is a line graph of the enrichment of NF-κB target genes in DMSO or apilimod-treated, 8 hours, cDCs after 6 days of culture.

FIG. 14D is a western blot showing the total histone H3, loading control, p-NF-κB-p65, and total NF-κB-p65 in PIKfyve WT vs. KO cDC lysates after 9 days of cultures and in DMSO or apilimod-treated, 20 hours, cDC lysates after 6 days of culture.

FIG. 14E is a western blot showing the total histone H3, loading control, p-IκB-α, IκB-α, and IκB-β in PIKfyve WT vs. KO cDC lysates after 9 days of culture and in DMSO or apilimod-treated, 20 hours, cDC lysates after 6 days of culture.

FIG. 14F is a western blot showing the total histone H3, loading control, IKK-α, IKK-β, and IKK-γ in PIKfyve WT vs. KO cDC lysates after 9 days of culture and in DMSO or apilimod-treated, 20 hours, cDC lysates after 6 days of culture.

FIG. 14G is a western blot showing the total histone H3, loading control, IKK-ε, p-TBK-1, and TBK-1 in PIKfyve WT vs. KO cDC lysates after 9 days of culture and in DMSO or apilimod-treated, 20 hours, cDC lysates after 6 days of culture.

FIG. 14H is a scatter plot of differentially expressed genes in apilimod versus DMSO-treated, 8 hours, cDCs after 6 days of culture.

FIG. 14I is a TCGA Pan-Cancer scatter plot of the correlation between SQSTM1 normalized gene expression and PIKfyve score.

FIG. 14J is a box plot of having high vs. low SQSTM1 for patients with high vs. low NF-κB gene targets expression or

FIG. 14K is a box plot of bulk RNA-seq data of having high vs. low SQSTM1 for patients with high vs. low cDC1 maturation score expression See, e.g., Ghislat, G. et al., Sci. Immunol. 2021:6.

FIG. 15A is a line graph of the subcutaneous tumor volume of MC38 tumors in PIKfyve KO mice or WT mice after 24 days.

FIG. 15B is a line graph of the subcutaneous tumor volume of MCA-205 tumors in PIKfyve KO mice or WT mice after 15 days.

FIG. 15C is a line graph of the subcutaneous tumor volume of B16F10 tumors in PIKfyve KO mice or WT mice after 15 days.

FIG. 15D is a line graph of the subcutaneous tumor volume of B16F10 tumors in PIKfyve KO mice or WT mice treated with anti-PD1 antibody, 200 μg every 3 days, or isotype control after 15 days.

FIG. 15E is a box and whisker plot of the XCR1 median fluorescent intensity by % cDC1 subset in B16F10 tumor model treated with vehicle or apilimod, 30 mg/kg daily.

FIG. 15F is a series of three box and whisker plot of the XCR1 median fluorescent intensity by all DCs, cDC1 or cDC2 subset in PIKfyve KO mice or WT mice treated with vehicle or apilimod, 30 mg/kg daily.

FIG. 15G is a box and whisker plot of the percentage of intracellular IL-12⁺ expression in WT vs. KO cDCs from tumor-draining lymph nodes of MC38 tumors after 20 hours of LPS, 20 ng/ml.

FIG. 15H is a box and whisker plot of the median fluorescent intensity MC38 tumor cDC1 subset in MHC-I tumors of PIKfyve KO or WT mice.

FIG. 15I is a box and whisker plot of the median fluorescent intensity MC38 tumor cDC1 subset in MHC-II tumors of PIKfyve KO or WT mice.

FIG. 15J is a box and whisker plot of the median fluorescent intensity MC38 tumor cDC1 subset in CD80 tumors of PIKfyve KO or WT mice.

FIG. 15K is a box and whisker plot of the median fluorescent intensity MC38 tumor cDC1 subset in CD86 tumors of PIKfyve KO or WT mice.

FIG. 15L is a box and whisker plot of the relative median fluorescent intensity of surface H2-kb-SIINFEKL in WT vs. KO cDCs from tumor-draining lymph nodes of B16F10-OVA tumors.

FIG. 15M is a box and whisker plot of the percentage of KSPWFTTL Tetramer⁺ CD8⁺ T cells isolated from MC38-OVA WT or KO tumors.

FIG. 15N is a series of two scatter plots of KSPWFTTL Tetramer⁺ CD8⁺ T cells isolated from MC38-OVA WT or KO tumors.

FIG. 16A is a line graph of the subcutaneous tumor volume of MC38 tumors in mice treated with vehicle or apilimod, 30 mg/kg×5 days/week.

FIG. 16B is a series of two scatter plots of the percent IFNγ⁺ of CD8⁺ T cells in vehicle or apilimod-treated MC38 tumors.

FIG. 16C is a line graph of the subcutaneous tumor volume of MC38 tumors in mice treated with vehicle versus apilimod, 30 mg/kg×5 days/week, in wild-type or Batf3^{-/-} mice.

FIG. 16D is a line graph of the subcutaneous tumor volume of B16F10 tumors in mice treated with vehicle or apilimod, 30 mg/kg daily, in wild-type or Batf3^{-/-} mice.

FIG. 16E is an experimental timeline of the vaccination experiment schedule demonstrating pre-treatment with oral, vehicle or apilimod, or subcutaneous, water or PolyI:C, reagents followed by B16F10-OVA tumor inoculation.

FIG. 16F is a series of four line graphs of the Individual growth curves of subcutaneous B16F10-OVA tumor volume following 21 days of pre-treatment with vehicle versus apilimod, 30 mg/kg daily, with or without subcutaneous injection of water versus PolyI:C, 100 μg on Day 1 and Day 14.

FIG. 16G is an experimental timeline of the vaccination therapy experiment demonstrating combination therapy treatment with oral, vehicle or apilimod, or subcutaneous, water or PolyI:C, reagents following B16F10-OVA tumor inoculation.

FIG. 16H is a series of four line graphs of the Individual growth curves of subcutaneous B16F10-OVA tumor volume in mice treated with vehicle or apilimod, 30 mg/kg daily, with or without a subcutaneous injection of water versus PolyI:C, 100 μg once weekly.

FIG. 17A is a box and whisker plot of the PIKfyve scores in patients with melanoma who were ICB treatment-naïve (“NAÏVE”) versus those who had previously progressed on ICB treatment (“PROG”) See, e.g., Riaz, N. et al., Cell 2017:171: 934-949.

FIG. 17B is a box plot of the low neoantigen load, disease stage, reference is MO, and mutation subtype, reference is None, from bulk RNA-seq data See, e.g., Riaz, N. et al., Cell 2017:171: 934-949.

FIG. 17C is a box plot of the hazard ratios of PROG cohort by high vs. low PIKfyve score, high vs. low neoantigen load, disease stage, reference is MIA, and mutation subtype, reference is None, from bulk RNA-seq data See, e.g., Riaz, N. et al., Cell 2017:171: 934-949.

FIG. 17D is a scatter plot of immune cells in pre-treatment tumors from patients with breast cancer, based on scRNA-seq data See, e.g., Qian, J. et al., Cell Res. 2020:30: 745-762.

FIG. 17E is a scatter plot of immune cells in pre-treatment tumors from patients with colorectal cancer, based on scRNA-seq data See, e.g., Qian, J. et al., Cell Res. 2020:30: 745-762.

FIG. 17F is a scatter plot of immune cells in pre-treatment tumors from patients with lung cancer, based on scRNA-seq data See, e.g., Qian, J. et al., Cell Res. 2020:30: 745-762.

FIG. 17G is a scatter plot of immune cells in pre-treatment tumors from patients with ovarian cancer, based on scRNA-seq data See, e.g., Qian, J. et al., Cell Res. 2020:30: 745-762.

FIG. 17H is a bar plot of PIKfyve cluster average expression by cancer, stroma, or immune cell type based on scRNA-seq data See, e.g., Qian, J. et al., Cell Res. 2020:30: 745-762.

FIG. 17G is a scatter plot of immune cells in pre-treatment tumors from patients with endometrial cancer based on scRNA-seq data See, e.g., Chow, R. D. et al., Cancer Discov. 2023:13: 312-331.

FIG. 17J is a box and whisker plot of the log PIKfyve expression in individual cDCs in patients with endometrial cancer, who were nonresponders CR to ICB treatment See, e.g., Chow, R. D. et al., Cancer Discov. 2023:13: 312-331.

FIG. 18A is a series of nine scatter plots of the gating strategy for CD69⁺ effector, naïve, terminally differentiated, resident memory, central memory, and exhausted CD8⁺ T cells from spleens of non-tumor-bearing mice treated with vehicle or apilimod, 30 mg/kg daily, after 5 days of treatment.

FIG. 18B is a box and whisker plot comparison of the percentage of CD69⁺ effector CD8⁺ T cells from spleens of non-tumor-bearing mice treated with vehicle or apilimod, 30 mg/kg daily, after 5 days of treatment.

FIG. 18C is a box and whisker plot comparison of the percentage of naïve CD8⁺ T cells from spleens of non-tumor-bearing mice treated with vehicle or apilimod, 30 mg/kg daily, after 5 days of treatment.

FIG. 18D is a box and whisker plot comparison of the percentage of terminally differentiated CD8⁺ T cells from spleens of non-tumor-bearing mice treated with vehicle or apilimod, 30 mg/kg daily, after 5 days of treatment.

FIG. 18E is a box and whisker plot comparison of the percentage of resident memory CD8⁺ T cells from spleens of non-tumor-bearing mice treated with vehicle or apilimod, 30 mg/kg daily, after 5 days of treatment.

FIG. 18F is a box and whisker plot comparison of the percentage of central memory CD8⁺ T cells from spleens of non-tumor-bearing mice treated with vehicle or apilimod, 30 mg/kg daily, after 5 days of treatment.

FIG. 18G is a box and whisker plot comparison of the percentage of exhausted CD8⁺ T cells from spleens of non-tumor-bearing mice treated with vehicle or apilimod, 30 mg/kg daily, after 5 days of treatment.

FIG. 18H is a series of nine scatter plots of the gating strategy for FIG. 13A-B and FIG. 18I for myeloid cells and cDCs.

FIG. 18I is a box and whisker plot comparison of the percentage of total CD11c in CD45⁺ cells from spleens of non-tumor-bearing mice treated with vehicle or apilimod, 30 mg/kg daily, after 5 days of treatment.

FIG. 18J is a box and whisker plot comparison of the percentage of total F4/80 in CD45⁺ cells from spleens of non-tumor-bearing mice treated with vehicle or apilimod, 30 mg/kg daily, after 5 days of treatment.

FIG. 18K is a box and whisker plot comparison of the percentage of total CD11b in CD45⁺ cells from spleens of non-tumor-bearing mice treated with vehicle or apilimod, 30 mg/kg daily, after 5 days of treatment.

FIG. 18L is a box and whisker plot comparison of the percentage of total cDC2 cells from spleens of non-tumor-bearing mice treated with vehicle or apilimod, 30 mg/kg daily, after 5 days of treatment.

FIG. 19A is a series of three scatter plots of the gating strategy for FIG. 13C-R and FIG. 18B-H, CD11c⁺ gate.

FIG. 19B is a line graph of representative density plot of median fluorescent intensity of surface XCR1 in DMSO or apilimod-treated, 20 hours, cDCs after 6 days of culture.

FIG. 19C a western blot showing total histone H3, loading control, and PIKfyve in Pikfyve WT vs. KO cDC lysates after 9 days of culture.

FIG. 19D is a box and whisker plot comparison of the percentage of CD80⁺CD86⁺⁻ cDCs in PIKfyve WT vs KO models.

FIG. 19E is a line graph (Left) and box and whisker plot (Right) of relative median fluorescent intensity of surface H-2kb-SIINFEKL in Pikfyve WT vs. KO cDC with or without 12 hours of pOVA, 100 ng/ml treatment after 9 days of culture.

FIG. 19F is a scatter plot (Left) and box and whisker plot (Right) of relative median fluorescent intensity of percent H-2kb-SIINFEKL⁺ cells in Pikfyve WT vs. KO cDC with or without 12 hours of pOVA, 100 ng/ml treatment after 9 days of culture.

FIG. 19G is a line graph (Left) and box and whisker plot (Right) of relative median fluorescent intensity of surface H-2kb-SIINFEKL in DMSO or apilimod-treated cDCs with or without 12 hours of pOVA, 100 ng/ml, treatment.

FIG. 19H is a scatter plot (Left) and box and whisker plot (Right) of relative median fluorescent intensity of percent H2kb-SIINFEKL⁺ cells in DMSO or apilimod-treated cDCs with or without 12 hours of pOVA, 100 ng/ml, treatment.

FIG. 20A is a series of three scatter plots of the CD90⁺ CD8⁺ gating strategy for (OT-I cells.

FIG. 20B is a scatter plot (Left) and box and whisker plot (Right) of percentage of IFNγ⁺ and OT-I cells after 48 hours of co-culture with Pikfyve WT vs. KO cDCs with or without pOVA, 100 ng/ml.

FIG. 20C is a scatter plot (Left) and box and whisker plot (Right) of percentage of granzyme B⁺ OT-I cells after 48 hours of co-culture with Pikfyve WT vs. KO cDCs with or without pOVA, 100 ng/ml.

FIG. 20D is a scatter plot (Left) and box and whisker plot (Right) of percentage of IFNγ⁺ OT-I cells after 48 hours of co-culture with DMSO or apilimod pre-treated cDCs with or without pOVA, 100 ng/ml.

FIG. 20E is a scatter plot (Left) and box and whisker plot (Right) of percentage of granzyme B⁺ OT-I cells after 48 hours of co-culture with DMSO or apilimod pre-treated cDCs with or without pOVA, 100 ng/ml.

FIG. 20F is a box and whisker plot of the percentage of Ki67⁺ OT-I cells after 72 hours of co-culture with Pikfyve WT vs. KO pre-treated cDCs with or without sOVA, 10 μg/ml.

FIG. 20G is a box and whisker plot of the percentage of Ki67⁺ OT-II cells after 72 hours of co-culture with Pikfyve WT vs. KO pre-treated cDCs with or without sOVA, 10 μg/ml.

FIG. 20H is a box and whisker plot of the percent of Ki67⁺ OT-I cells after 72 hours of co-culture with DMSO or apilimod pre-treated cDCs with or without sOVA, 10 μg/ml.

FIG. 20I is a box and whisker plot of the percent of Ki67⁺ OT-II cells after 72 hours of co-culture with DMSO or apilimod pre-treated cDCs with or without sOVA, 10 μg/ml.

FIG. 21A is a line graph of Dendritic Cell Maturation Up gene signature (M4562: LENAOUR_DENDRITIC_CELL_MATURATION_UP) in PIKfyve KO vs. WT cDCs after 9 days of culture.

FIG. 21B is a line graph of MSigDB Hallmark “TNF_SIGNALING_VIA_NFκB” in PIKfyve KO vs. WT cDCs after 9 days of culture.

FIG. 21C is a line graph of MSigDB Hallmark “TNF_SIGNALING_VIA_NFκB” in apilimod vs. DMSO-treated, 3 hours, cDCs after 6 days of culture.

FIG. 21D is a line graph of MSigDB Hallmark “TNF_SIGNALING_VIA_NFκB” in apilimod vs. DMSO-treated, 8 hours, cDCs after 6 days of culture.

FIG. 21E is a bar graph comparison of Il12b expression fold change by RT-qPCR in apilimod vs. DMSO-treated, 3 or 12 hours, cDCs after 6 days of culture.

FIG. 21F is a bar graph comparison of IL-12p40 protein levels by ELISA in media from apilimod vs. DMSO-treated, 12 hours, cDCs after 6 days of culture.

FIG. 21G is a bar graph Supp. 5 g Comparison of IL-12p70 protein levels by ELISA in media from apilimod vs. DMSO-treated, 24 hours, cDCs after 6 days of culture.

FIG. 22A is a line graph of the overall survival of patients in the TCGA Pan-Cancer bulk RNA-seq dataset, by median high or low SQSTM1 normalized gene expression.

FIG. 22B is a box and whisker plot of the comparison of SQSTM1 normalized gene expression across myeloid lineage cell types. Data plotted are mean±s.d. from bulk RNA-seq data See, e.g., Murakami, K. et al., Nat. Immunol. 2021:22: 301-311.

FIG. 22C is a scatter plot of the correlation between SQSTM1 normalized gene expression and NF-κB target genes expression in the TCGA Pan-Cancer bulk RNA-seq dataset.

FIG. 23A is a line graph of the tumor weights for MC38 tumor measured on Day 26.

FIG. 23B is a line graph of the tumor weights for MCA-205 tumor measured on Day 16.

FIG. 23C is a line graph of the tumor weights for B16F10 tumor measured on Day 16.

FIG. 23D is a series of six scatter plots of the gating strategy for dendritic cells from tumors and tumor-draining lymph nodes (TDLN), CD45⁺ Lin(−) CD11c⁺ MHC-II⁺ gate, based on FIG. 15E-L, FIG. 23E-O, and FIG. 25D-E.

FIG. 23E is a scatter plot of the gating strategy for additional cDC1 and cDC2 gates for dendritic cells from tumors based on FIG. 15E-K, FIG. 23F-O.

FIG. 23F is a box and whisker plot of the % cDC2 of all DC cells in PIKfyve KO mice or WT mice treated with vehicle or apilimod, 30 mg/kg daily.

FIG. 23G is a series of three box and whisker plots of the SIRP1α median fluorescent intensity by all DC, cDC1 or cDC2 subset in PIKfyve KO mice or WT mice treated with vehicle or apilimod, 30 mg/kg daily.

FIG. 23H is a box and whisker plot of the MHC-I median fluorescent intensity in the cDC2 subset tumors of PIKfyve KO or WT mice.

FIG. 23I is a box and whisker plot of the MHC-II median fluorescent intensity in the cDC2 subset tumors of PIKfyve KO or WT mice.

FIG. 23J is a box and whisker plot of the CD80 median fluorescent intensity in the cDC2 subset tumors of PIKfyve KO or WT mice.

FIG. 23K is a box and whisker plot of the CD86 median fluorescent intensity in the cDC2 subset tumors of PIKfyve KO or WT mice.

FIG. 23L is a box and whisker plot of the MHC-I median fluorescent intensity in all DCs in tumors of PIKfyve KO or WT mice.

FIG. 23M is a box and whisker plot of the MHC-II median fluorescent intensity in all DCs in tumors of PIKfyve KO or WT mice.

FIG. 23N is a box and whisker plot of the MHC-I CD80 median fluorescent intensity in all DCs in tumors of PIKfyve KO or WT mice.

FIG. 23O is a box and whisker plot of the CD86 median fluorescent intensity in all DCs in tumors of PIKfyve KO or WT mice.

FIG. 24A is a box and whisker plot of the comparison percentages of CXCR3⁺CD8⁺ T cells isolated from tumors of PIKfyve KO or WT mice.

FIG. 24B is a box and whisker plot of the comparison percentages of CXCR3⁺CD4⁺ T cells isolated from tumors of PIKfyve KO or WT mice.

FIG. 24C is a box and whisker plot of the comparison percentages of CD69⁺CD8⁺ T cells isolated from tumors of PIKfyve KO or WT mice.

FIG. 24D is a box and whisker plot of the comparison percentages of CD69⁺CD4⁺ T cells isolated from tumors of PIKfyve KO or WT mice.

FIG. 24E is a box and whisker plot of the comparison percentages of CD44^{Hi}CD8⁺ T cells isolated from tumors of PIKfyve KO or WT mice.

FIG. 24F is a box and whisker plot of the comparison percentages of CD44^{Hi}CD4⁺ T cells isolated from tumors of PIKfyve KO or WT mice.

FIG. 24G is a box and whisker plot of the comparison percentages of CD62L⁺CD8⁺ T cells isolated from tumors of PIKfyve KO or WT mice.

FIG. 24H is a box and whisker plot of the comparison percentages of CD62L⁺CD4⁺ T cells isolated from tumors of PIKfyve KO or WT mice.

FIG. 24I is a box and whisker plot of the comparison percentages of KLRG1⁺CD8⁺ T cells isolated from tumors of PIKfyve KO or WT mice.

FIG. 24J is a box and whisker plot of the comparison percentages of KLRG1⁺CD4⁺ T cells isolated from tumors of PIKfyve KO or WT mice.

FIG. 24K is a box and whisker plot of the comparison percentages of PD-1⁺ CD8⁺ T cells isolated from tumors of PIKfyve KO or WT mice.

FIG. 24L is a box and whisker plot of the comparison percentages of PD-1⁺CD4⁺ T cells isolated from tumors of PIKfyve KO or WT mice.

FIG. 24M is a box and whisker plot of the comparison percentages of TIM-3⁺CD8⁺ T cells isolated from tumors of PIKfyve KO or WT mice.

FIG. 24N is a box and whisker plot of the comparison percentages of TIM-3⁺CD4⁺ T cells isolated from tumors of PIKfyve KO or WT mice.

FIG. 25A is a line graph of the subcutaneous tumor volume Experiment 1 of B16F10-OVA tumors in PIKfyve KO mice or WT mice.

FIG. 25B is a line graph of the subcutaneous tumor volume Experiment 2 of B16F10-OVA tumors in PIKfyve KO mice or WT mice.

FIG. 25C is a series of two images of bilateral subcutaneous B16F10-OVA tumors were excised from 3 PIKfyve KO mice or WT mice per Experiment 1 and 2.

FIG. 25D is a box and whisker plot of the percent dendritic cells of CD45⁺ cells in PIKfyve WT vs. KO tumor-draining lymph nodes of B16F10-OVA tumors.

FIG. 25E is a box and whisker plot of the percent H2-kb-SIINFEKL⁺ dendritic cells in WT vs. KO tumor-draining lymph nodes of B16F10-OVA tumors.

FIG. 25F is a series of five scatter plots of the gating strategy for 7-AAD⁻ CD90⁺ CD3⁺CD8⁺ gate for intratumoral CD8⁺ T cells based on FIG. 15M and FIG. 25G-I).

FIG. 25G is a box and whisker plot of the percentage of SIINFEKL Tetramer⁺ CD8⁺ T cells isolated from subcutaneous B16F10-OVA WT or KO tumors.

FIG. 25H is a series of six scatter plots of SIINFEKL Tetramer⁺ CD8⁺ T cells isolated from subcutaneous B16F10-OVA WT or KO tumors.

FIG. 25I is a box and whisker plot of the percentage of SIINFEKL Tetramer⁺ CD8⁺ T cells isolated from MC38-OVA WT or KO tumors.

FIG. 25J is a series of two scatter plots of SIINFEKL Tetramer⁺ CD8⁺ T cells isolated from MC38-OVA WT or KO tumors.

FIG. 26A is a bar graph of the percent IFNγ⁺ of CD8⁺ T cells in vehicle or apilimod-treated, 30 mg/kg×5 days/week MC38 tumors.

FIG. 26B is a line graph of the subcutaneous tumor volume of MC38 tumors treated with vehicle versus apilimod, 30 mg/kg×5 days/week, in NSG™ mice.

FIG. 26C is a line graph of the subcutaneous tumor volume of MC38 tumors in mice treated with vehicle versus apilimod, 30 mg/kg×5 days/week, and additionally treated with IgG2a or αIL-12p40 blocking antibody.

FIG. 26D is a line graph of the subcutaneous tumor volume of MC38 tumors in mice treated with vehicle versus apilimod, 30 mg/kg×5 days/week, and additionally treated with IgG1 or αIFNγ blocking antibody.

FIG. 26E is a line graph of the subcutaneous tumor volume of B16F10 tumors in mice treated with vehicle or apilimod, 30 mg/kg daily, in NSG™ mice.

FIG. 26F is a line graph of the subcutaneous tumor volume of B16F10 tumors in mice treated with vehicle versus apilimod, 30 mg/kg daily, in NSG™ mice.

FIG. 26G is a line graph of the relative median fluorescent intensity of the surface MHC-I, H2-kb, H2-kd, on DMSO or apilimod-treated cDCs with or without PolyI:C, 50 μg/ml or LPS, 50 ng/ml, treatment, 20 hours, after 6 days of culture.

FIG. 26H is a line graph of the relative median fluorescent intensity of the surface MHC-II, MHC-IA-IE, on DMSO or apilimod-treated cDCs with or without PolyI:C, 50 μg/ml or LPS, 50 ng/ml, treatment, 20 hours, after 6 days of culture.

FIG. 26I is a line graph of the subcutaneous tumor volume of B16F10-OVA tumors following 21 days of pre-treatment with vehicle versus apilimod, 30 mg/kg×5 days/week, with or without subcutaneous injection of water versus PolyI:C, 100 μg on Day 1 and Day 14.

FIG. 26J is a line graph of the subcutaneous tumor volume of B16F10-OVA tumors in mice treated with vehicle or apilimod, 30 mg/kg×5 days/week, with or without subcutaneous injection of water versus PolyI:C, 100 μg once weekly.

### DETAILED DESCRIPTION

Applicant has discovered that inhibition of PIKfyve with ESK981 and/or apilimod upregulates surface expression of MHC-I in cancer cells resulting in enhanced cancer cell killing. The antitumor responses by Pikfyve inhibition was found to be CD8⁺ T cell- and MHC-I-dependent. In addition, inhibition of PIKfyve improved the response to ACT and therapeutic vaccine administration in pre-clinical models. High expression of PIKfyve was also predictive of poor response to ICB and prognostic of poor survival in ICB-treated patient cohorts. Collectively, these results show that inhibiting PIKfyve surprising increases immunotherapy response in cancer subjects.

Additionally, the applicant has discovered that among shared gene targets of clinically relevant protein kinase inhibitors, high PIKfyve expression was least predictive of complete response in patients who received immune checkpoint blockade (ICB). In immune cells, high PIKfyve expression in DCs was associated with worse response to ICB. Genetic and pharmacological studies demonstrated that PIKfyve ablation enhanced DC function via selectively altering the alternate/non-canonical NF-κB pathway. Both loss of Pikfyve in DCs and treatment with apilimod, a potent and specific PIKfyve inhibitor, restrained tumor growth, enhanced DC-dependent T cell immunity, and potentiated ICB efficacy in tumor-bearing mouse models. Furthermore, the combination of a vaccine adjuvant and apilimod reduced tumor progression in vivo. Thus, PIKfyve negatively controls DCs, and PIKfyve inhibition has promise for cancer immunotherapy and vaccine treatment strategies.

In one embodiment, the present disclosure provides a method of treating cancer in a subject in need thereof, the method comprising administering a therapeutically effective amount of ESK981 and/or apilimod, i.e., (a) ESK981 alone; or (b) apilimod alone; or (c) ESK981 and apilimod, to the subject in combination with a therapeutically effective amount of an immunotherapy.

In another embodiment, the immunotherapy is CD8⁺ T cell- and major histocompatibility complex (MHC) class I-dependent immunotherapy.

In another embodiment, the immunotherapy is adoptive cell therapy (ACT) or a personalized cancer vaccine.

In another embodiment, the immunotherapy is an adoptive cell therapy.

In another aspect, the immunotherapy is a personalized cancer vaccine.

In another aspect, the immunotherapy is immune checkpoint blockade.

In another embodiment, the adoptive cell therapy comprises T cells, dendritic cells, macrophages, peripheral blood mononuclear cells (PBMCs), or a combination thereof.

In another embodiment, the adoptive cell therapy comprises T cells. In another embodiment, the adoptive cell therapy comprises engineered cells expressing a heterologous T cell receptor, a chimeric antigen receptor, or a combination thereof.

In another embodiment, the adoptive cell therapy comprises engineered cells expressing a heterologous T cell receptor.

In another embodiment, the heterologous T cell receptor comprises an extracellular binding moiety that specifically binds a tumor-associated antigen.

In another embodiment, the heterologous T cell receptor comprises an anti-CD3 binding moiety.

In another embodiment, the heterologous T cell receptor comprises an anti-CD28 binding moiety.

In another embodiment, the heterologous T cell receptor comprises an extracellular binding moiety, a transmembrane domain, and an intracellular domain that triggers the activation, proliferation, or both of lymphocytes.

In another embodiment, the intracellular domain comprises a CD3 signaling domain. In another embodiment, the intracellular domain comprises a CD28 signaling domain.

In another embodiment, the heterologous T cell receptor comprises at least one costimulatory domain.

In another embodiment, the heterologous T cell receptor comprises a CD28 costimulatory domain.

In another embodiment, the immunotherapy is a personalized cancer vaccine.

In another embodiment, personalized cancer vaccine comprises a phagocytosis stimulating agent, an immunostimulatory adjuvant, and attenuated cancer cells.

In another embodiment, the attenuated cancer cells are obtained from a tumor of the subject.

In another embodiment, the immunostimulatory adjuvant comprises a Toll like receptor (TLR) agonist.

In another embodiment, the immunostimulatory adjuvant comprises an anti-CD3 antibody.

In another embodiment, the immunostimulatory adjuvant comprises an anti-CD28 antibody.

In another embodiment, the cancer is or has become resistant to immunotherapy in the absence of ESK981 or apilimod.

In another embodiment, the cancer is any cancer listed in Table 1 and/or Table 2.

In another embodiment, the present disclosure provides a method of treating cancer in a subject in need thereof, the method comprising administering a therapeutically effective amount of ESK981 and/or apilimod to the subject in combination with a therapeutically effective amount of an immunotherapy, wherein the cancer is characterized as having an overexpression of PIKfyve.

In another embodiment, the present disclosure provides a method of treating cancer in a subject, the method comprising:
(a) determining whether an overexpression of PIKfyve is present or absent in a biological sample taken from the subject; and
(b) administering a therapeutically effective amount of ESK981 and/or apilimod in combination with an immunotherapy to the subject if an overexpression of PIKfyve is present in the biological sample.

In another embodiment, the present disclosure provides a method, comprising administering a therapeutically effective amount of ESK981 and/or apilimod to a subject in need thereof, wherein:
(a) the subject has cancer; and
(b) the cancer is characterized as having an overexpression of PIKfyve.

In another embodiment, the present disclosure provides a method of identifying whether a subject having cancer as a candidate for treatment with ESK981 and/or apilimod, the method comprising:
(a) determining whether an overexpression of PIKfyve is present or absent in a biological sample taken from the subject; and
(b) identifying the subject as being a candidate for treatment if an overexpression of PIKfyve is present; or
(c) identifying the subject as not being a candidate for treatment if an overexpression of PIKfyve is absent.

In another embodiment, the present disclosure provides a method of predicting treatment outcome in a subject having cancer, the method comprising determining whether an overexpression of PIKfyve is present or absent in a biological sample taken from the subject, wherein:
(a) the presence of an overexpression of PIKfyve in the biological sample indicates that administering ESK981 and/or apilimod in combination with an immunotherapy to the subject will likely cause a favorable therapeutic response; and
(b) the absence of an overexpression of PIKfyve in the biological sample indicates that administering ESK981 and/or apilimod in combination with an immunotherapy to the subject will likely cause an unfavorable therapeutic response.

In another embodiment, the present disclosure provides a method of treating cancer in a subject in need thereof comprising administering a therapeutically effective amount of a PIKfyve inhibitor to the subject in combination with a therapeutically effective amount of ACT or a therapeutically effective amount of a therapeutic cancer vaccine.

In another embodiment, the present disclosure provides a method of treating cancer in a subject in need thereof comprising administering a therapeutically effective amount of ESK981 to the subject in combination with a therapeutically effective amount of ACT or a therapeutically effective amount of a therapeutic cancer vaccine.

In another embodiment, the present disclosure provides a method of treating cancer in a subject in need thereof comprising administering a therapeutically effective amount of apilimod to the subject in combination with a therapeutically effective amount of ACT or a therapeutically effective amount of a therapeutic cancer vaccine.

In some embodiments, ESK981 and/or apilimod, and an immunotherapy are administered to a subject under one or more of the following conditions: at different periodicities, at different durations, at different concentrations, by different administration routes, etc. In some embodiments, for example, ESK981 and/or apilimod is administered prior to the immunotherapy, e.g., 0.5, 1, 2, 3, 4, 5, 10, 12, or 18 hours, 1, 2, 3, 4, 5, or 6 days, or 1, 2, 3, or 4 weeks prior to the administration of the immunotherapy. In some embodiments, ESK981 and/or apilimod is administered after the immunotherapy, e.g., 0.5, 1, 2, 3, 4, 5, 10, 12, or 18 hours, 1, 2, 3, 4, 5, or 6 days, or 1, 2, 3, or 4 weeks after the administration of the immunotherapy. In some embodiments, ESK981 and/or apilimod and the immunotherapy are administered concurrently but on different schedules, e.g., ESK981 is administered daily while the immunotherapy is administered once a week, once every two weeks, once every three weeks, or once every four weeks.

In some embodiments, compositions within the scope of this disclosure include compositions wherein ESK981 and/or apilimod is administered in an amount which is effective to achieve its intended purpose. While individual needs vary, determination of optimal ranges of effective amounts of each component is within the skill of the art. Typically, ESK981 and/or apilimod may be administered to subjects, e.g., human cancer patients, orally at a dose of 0.0025 to 100 mg/kg, or an equivalent amount of the pharmaceutically acceptable salt thereof, per day of the body weight of the mammal being treated for disorders responsive to induction of apoptosis. In one embodiment, for example, about 0.01 to about 100 mg/kg of ESK981 is orally administered to treat, ameliorate, or prevent cancer, e.g., prostate cancer or pancreatic cancer, e.g., NEPC, PDAC, PNETs, or NECs. For intramuscular injection, the dose is generally about one-half of the oral dose. For example, a suitable intramuscular dose would be about 0.0025 to about 25 mg/kg, or from about 0.01 to about 5 mg/kg.

In some embodiments, the unit oral dose may comprise from about 0.01 to about 1000 mg, for example, about 0.1 to about 100 mg of ESK981 or apilimod. The unit dose may be administered one or more times daily as one or more tablets or capsules each containing from about 0.1 to about 10 mg or about 0.25 to 50 mg of ESK981 or apilimod.

In some embodiments, for example, ESK981 may be present at a concentration of about 0.01 to 100 mg per gram of carrier. In an embodiment, ESK981 is present at a concentration of about 0.07-1.0 mg/ml, for example, about 0.1-0.5 mg/ml, and in one embodiment, about 0.4 mg/ml.

In some embodiments, in addition to administering ESK981 and/or apilimod as a raw chemical, these compounds may be administered as part of a pharmaceutical formulation containing suitable pharmaceutically acceptable carriers comprising excipients and auxiliaries, which facilitate the processing of these compounds into preparations, which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01% to 99%, in one embodiment from about 0.25% to 75% of ESK981 or apilimod, together with the excipient.

In some embodiments, the pharmaceutical compositions comprising ESK981 or apilimod may be administered to any subject which may experience the beneficial effects of these drugs. Foremost among such subjects are mammals, e.g., humans, e.g., human cancer patients, although the present disclosure is not intended to be so limited. Other subjects include veterinary animals (cows, sheep, pigs, horses, dogs, cats, and the like).

ESK981 and/or apilimod, and pharmaceutical compositions thereof, may be administered to a subject by any means that achieve their intended purpose. For example, administration may be by parenteral, subcutaneous, intravenous, intramuscular, intraperitoneal, transdermal, buccal, intrathecal, intracranial, intranasal, or topical routes. Alternatively, or concurrently, administration may be by the oral route. The dosage administered will be dependent upon the age, health, and weight of the recipient, kind of concurrent treatment, if any, frequency of treatment, and the nature of the effect desired.

In some embodiments, the pharmaceutical preparations of the present disclosure are manufactured in a manner which is itself known, for example, by means of conventional mixing, granulating, dragee-making, dissolving, or lyophilizing processes. Thus, pharmaceutical preparations for oral use can be obtained, for example, by combining ESK981 with solid excipients, optionally grinding the resulting mixture, and processing the mixture of granules, after adding suitable auxiliaries, if desired or necessary, to obtain tablets or dragee cores.

In some embodiments, suitable excipients are, in particular, fillers such as saccharides, for example, lactose or sucrose, mannitol or sorbitol, cellulose preparations and/or calcium phosphates, for example, tricalcium phosphate or calcium hydrogen phosphate, as well as binders such as starch paste, using, for example, maize starch, wheat starch, rice starch, potato starch, gelatin, tragacanth, methylcellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid, or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions, and suitable organic solvents or solvent mixtures. In order to produce coatings resistant to gastric juices, solutions of suitable cellulose preparations such as acetylcellulose phthalate or hydroxypropylmethylcellulose phthalate, are used. Dye stuffs or pigments may be added to the tablets or dragee coatings, for example, for identification or in order to characterize combinations of active compound doses.

In some embodiments, other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

In some embodiments, possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

In some embodiments, suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides, or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

In some embodiments, the topical compositions of this disclosure are formulated in one embodiment as oils, creams, lotions, ointments, and the like by choice of appropriate carriers. Suitable carriers include vegetable or mineral oils, white petrolatum (white soft paraffin), branched chain fats or oils, animal fats, and high molecular weight alcohol (greater than C₁₂). The carriers may be those in which the active ingredient is soluble. Emulsifiers, stabilizers, humectants, and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

In some embodiments, ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

In some embodiments, the present disclosure provides methods of treating a subject having cancer comprising (a) determining whether a biomarker is present or absent in a biological sample taken from the subject; and (b) administering a therapeutically effective amount of ESK981 and/or apilimod in combination with an immunotherapy to the subject if the biomarker is present in the biological sample See, e.g., Goossens et al., Transl Cancer Res. 4:256-269 (2015); Kamel and Al-Amodi, Genomics Proteomics Bioinformatics 15:220-235 (2017); and Konikova and Kusenda, Neoplasma 50:31-40 (2003). In some embodiments, the biomarker is an elevated expression level of PIKfyve.

In some embodiments, biomarker standards can be predetermined, determined concurrently, or determined after a biological sample is obtained from the subject. Biomarker standards for use with the methods described herein can, for example, include data from samples from subjects without cancer; data from samples from subjects with cancer, e.g., prostate cancer, that is not metastatic; and data from samples from subjects with cancer, e.g., prostate cancer and pancreatic cancer, that is metastatic. Comparisons can be made to establish predetermined threshold biomarker standards for different classes of subjects, e.g., diseased vs. undiseased subjects. The standards can be run in the same assay or can be known standards from a previous assay.

In some embodiments, a biomarker is differentially present between different phenotypic status groups if the mean or median expression or mutation levels of the biomarker is are calculated to be different, i.e., higher or lower, between the groups. Thus, biomarkers provide an indication that a subject, e.g., a cancer patient, belongs to one phenotypic status or another.

In some embodiments, the determination of the expression level or mutation status of a biomarker in a subject can be performed using any of the many methods known in the art. Any method known in the art for quantitating specific proteins and/or detecting the expression or mutation levels of PIKfyve in a subject or a biological sample may be used in the methods of the present disclosure. Examples include, but are not limited to, PCR (polymerase chain reaction), or RT-PCR, flow cytometry, Northern blot, Western blot, ELISA (enzyme linked immunosorbent assay), RIA (radioimmunoassay), gene chip analysis of RNA expression, immunohistochemistry or immunofluorescence See, e.g., Slagle et al., Cancer 83:1401 (1998); Hudlebusch et al., Clin Cancer Res 17:2919-2933 (2011). Certain embodiments of the present disclosure include methods wherein biomarker RNA expression (transcription) is determined. Other embodiments of the present disclosure include methods wherein protein expression in the biological sample is determined See, e.g., Harlow et al., Antibodies: A Laboratory Manual, Cold Spring Harbor Laboratory, Cold Spring Harbor, NY, (1988); Ausubel et al., Current Protocols in Molecular Biology, John Wiley & Sons, New York 3rd Edition, (1995); Kamel and Al-Amodi, Genomics Proteomics Bioinformatics 15:220-235 (2017). For northern blot or RT-PCR analysis, RNA is isolated from the tumor tissue sample using RNAse free techniques. Such techniques are commonly known in the art.

In one embodiment of the disclosure, a biological sample is obtained from the subject and the biological sample is assayed for PIKfyve expression.

The term “ESK981” refers to 13-isobutyl-4-methyl-10-(pyrimidin-2-ylamino)-1,2,4,7,8,13-hexahydro-6H-indazolo[5,4-a]pyrrolo[3,4-c]carbazol-6-one:

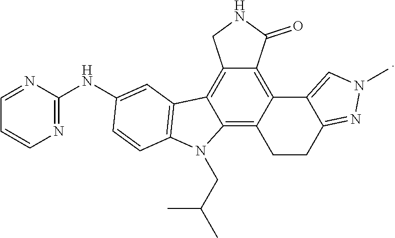 [SMILES: CC(C)Cn1c2ccc(Nc3ncccn3)cc2c2c3c(c4c(c21)CCc1nn(C)cc1-4)C(=O)NC3]

ESK981 (formerly known as CEP-11981) is an oral multi-tyrosine kinase inhibitor (MTKI) See, e.g., Hudkins et al., J. Med. Chem. 55:903-913 (2012); Invest New Drugs. 2014; 32(6):1258-68. ESK981 also inhibits autophagy through direct targeting of the lipid kinase PIKfyve See, e.g., Qiao et al., Nat Cancer. 2021; 2:978-93; WO 2022/094058.

The term “apilimod” refers to N-[(E)-(3-methylphenyl)methylideneamino]-6-morpholin-4-yl-2-(2-pyridin-2-ylethoxy)pyrimidin-4-amine:

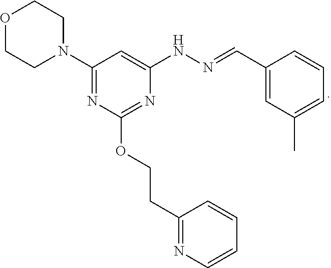 [SMILES: Cc1cccc(/C=N/Nc2cc(N3CCOCC3)nc(OCCc3ccccn3)n2)c1]

Apilimod (formerly known as STA-5326) is an interleukins IL-12 and IL-23 inhibitor that was initially developed for the treatment of autoimmune conditions like Crohn's disease and rheumatoid arthritis See, e.g., Billich A. IDrugs 2007:10(1):53-59. Apilimod also inhibits autophagy by inhibiting lipid kinase enzyme PIKfyve See, e.g., Shisheva, A. et al., Molec. and Cell. Bio. 1999; 64(6):1750-1755; Cai, X. Chem. & Bio. 2013; 20(7):912-921.

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of cancer, e.g., prostate cancer or pancreatic cancer, e.g., PDAC, PNETs, or NECs, in one embodiment, a therapeutically effective amount will refer to the amount of a therapeutic agent that decreases the rate of tumor growth, decreases tumor mass, decreases the number of metastases, increases time to tumor progression, or increases survival time by at least 1%, at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The term “hyperproliferative disease,” as used herein, refers to any condition in which a localized population of proliferating cells in an animal is not governed by the usual limitations of normal growth. Examples of hyperproliferative disorders include tumors, neoplasms, lymphomas and the like. A neoplasm is said to be benign if it does not undergo invasion or metastasis and malignant if it does either of these. A “metastatic” cell means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell.

The term “normal cell,” as used herein, refers to a cell that is not undergoing abnormal growth or division. Normal cells are non-cancerous and are not part of any hyperproliferative disease or disorder.

The terms “a” and “an” refer to one or more.

The term “about,” as used herein, includes the recited number±10%. Thus, “about 10” means 9 to 11.

The terms “treat,” “treating,” “treatment,” and the like as used herein refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of ESK981 to a subject in need of such treatment.

The terms “prevent,” “preventing,” and “prevention,” as used herein, refer to a decrease in the occurrence of pathological cells, e.g., hyperproliferative or neoplastic cells, in an subject. The prevention may be complete, e.g., the total absence of pathological cells in a subject. The prevention may also be partial, such that the occurrence of pathological cells in a subject is less than that which would have occurred without the present disclosure.

The term “biological sample” as used herein refers any tissue or fluid from a subject that is suitable for detecting a biomarker, e.g., PIKfyve expression. Examples of useful biological samples include, but are not limited to, biopsied tissues and/or cells, e.g., solid tumor, lymph gland, inflamed tissue, tissue and/or cells involved in a condition or disease, blood, plasma, serous fluid, cerebrospinal fluid, saliva, urine, lymph, cerebral spinal fluid, and the like. Other suitable biological samples will be familiar to those of ordinary skill in the relevant arts. A biological sample can be analyzed for genetic aberrations using any technique known in the art. Such techniques include, but are not limited to, polymerase chain reaction (PCR) methodology, reverse transcription-polymerase chain reaction (RT-PCR) methodology, or cytoplasmic light chain immunofluorescence combined with fluorescence in situ hybridization (cIg-FISH). A biological sample can be obtained using techniques that are well within the scope of ordinary knowledge of a clinical practioner. In one embodiment of the present disclosure, the biological sample comprises blood cells.

The phrase “in combination” as used in connection with the administration of (i) ESK981 and/or apilimod; and (ii) an immunotherapy, e.g., ACT or a personalized cancer vaccine, to a subject means that ESK981 and/or apilimod, and the immunotherapy can be administered to the subject together, e.g., as part of a single pharmaceutical composition or formulation, or separately, e.g., as part of two or more separate pharmaceutical compositions or formulations. The phrase “in combination” as used in this context is thus intended to embrace, for example, administration of ESK981 and an immunotherapy in a sequential manner, wherein ESK981 and the immunotherapy are administered to the subject at different times, e.g., 1 hour, 1 day, or 1 week apart, as well as administration concurrently, or in a substantially simultaneous manner. Sequential or substantially simultaneous administration of the ESK981 and the immunotherapy can be accomplished by any appropriate route including, but not limited to, oral routes, intravenous routes, intramuscular routes, and direct absorption through mucous membrane tissues. ESK981 and the immunotherapy can be administered by the same route or by different routes. For example, an immunotherapy may be administered by intravenous injection while ESK981 of the combination may be administered orally. ESK981 and an immunotherapy may also be administered in alternation. In one embodiment, ESK91 and the immunotherapy are administered to a subject separately, e.g., as par of two or more separate pharmaceutical compositions or formulations. In one embodiment, ESK91 and the immunotherapy are administered to a subject according to different dosing schedules, e.g., ESK981 is administered daily and the immunotherapy is administered once a week, once a month, etc.

In some embodiments, the present disclosure provides a method of treating cancer in a subject comprising administering a therapeutically effective amount of ESK9-1 and/or apilimod in combination with an immune checkpoint inhibitor, an adoptive cell therapy (ACT), or a personalized cancer vaccine. While not being limited to a specific mechanism, in some embodiments, the combinations of the present disclosure treat cancer at least in part by inhibiting PIKfyve. Examples of treatable cancers include, but are not limited to, any one or more of the cancers of Table 1.

TABLE 1

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic fibroma |
| lymphoma | rhabdomyosarcoma | sarcoma | |
| anaplastic large | anaplastic thyroid | angioimmunoblastic | angiomyolipoma |
| cell lymphoma | cancer | T-cell lymphoma | |
| angiosarcoma | astrocytoma | atypical teratoid | B-cell chronic |
| | | rhabdoid tumor | lymphocytic leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial tumor | |
| embryonal | endocrine gland | endodermal sinus | enteropathy- |
| carcinoma | neoplasm | tumor | associated T-cell |
| | | | lymphoma |
| esophageal cancer | fetus in fetu | fibroma | fibrosarcoma |
| follicular | follicular thyroid | ganglioneuroma | gastrointestinal cancer |
| lymphoma | cancer | | |
| germ cell tumor | gestational | giant cell | giant cell tumor of the |
| | choriocarcinoma | fibroblastoma | bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck | hemangiopericytoma | hematological cancer | hepatoblastoma |
| cancer | | | |
| hepatosplenic T- | Hodgkin's lymphoma | non-Hodgkin's | invasive lobular |
| cell lymphoma | | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic lymphocytic |
| | leukemia | leukemia | leukemia |
| liver cancer | small cell lung cancer | non-small cell lung | MALT lymphoma |
| | | cancer | |
| malignant fibrous | malignant peripheral | malignant triton tumor | mantle cell lymphoma |
| histiocytoma | nerve sheath tumor | | |
| marginal zone B- | mast cell leukemia | mediastinal germ cell | medullary carcinoma |
| cell lymphoma | | tumor | of the breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid | myxoma | myxosarcoma | nasopharyngeal |
| liposarcoma | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell | renal medullary | retinoblastoma | rhabdomyoma |
| carcinoma | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell carcinoma |
| carcinoma | | tumors | |
| soft tissue sarcoma | somatostatinoma | soot wart | spinal tumor |
| splenic marginal | squamous cell | synovial sarcoma | Sezary's disease |
| zone lymphoma | carcinoma | | |
| small intestine | squamous carcinoma | stomach cancer | T-cell lymphoma |
| cancer | | | |
| testicular cancer | thecoma | thyroid cancer | transitional cell |
| | | | carcinoma |
| throat cancer | urachal cancer | urogenital cancer | urothelial carcinoma |
| uveal melanoma | uterine cancer | verrucous carcinoma | visual pathway glioma |
| vulvar cancer | vaginal cancer | Waldenstrom's | Warthin's tumor |
| | | macroglobulinemia | |
| Wilms' tumor | | | |

In another embodiment, the cancer is a solid tumor. In another embodiment, the cancer a hematological cancer. Exemplary hematological cancers include, but are not limited to, the cancers listed in Table 2. In another embodiment, the hematological cancer is acute lymphocytic leukemia, chronic lymphocytic leukemia (including B-cell chronic lymphocytic leukemia), or acute myeloid leukemia.

TABLE 2

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic |
| | lymphoma |
| Waldenstrom's macroglobulinemia | T-cell lymphoma |
| (WM) | |
| diffuse large B-cell lymphoma | mast cell leukemia |
| (DLBCL) | |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell |
| | lymphoma |
| Richter's transformation | |

In some embodiments, the present disclosure provides a method of treating cancer in a subject comprising administering a therapeutically effective amount of ESK981 and/or apilimod in combination with adoptive cell therapy. Adoptive cell therapy is an immunotherapy using a subject's own immune cells (or a donor's immune cells) to treat diseases such as, for example, cancer. In adoptive cell therapy, T cells are isolated based upon their ability to expand in response to tumor or are genetically modified to target certain molecules on cells, such as antigens on cancer cells. The tumor reactive T cells are then expanded and infused back into the subject.

In some embodiments, one form of adoptive cell therapy called TIL therapy involves tumor infiltrating lymphocytes. In TIL therapy, tumor infiltrating lymphocytes that have penetrated a tumor are collected from a tumor biopsy taken from a subject and expanded in vitro for re-infusion into the patient. The tumor infiltrating lymphocytes are actively engaged in tumor destruction. In one method, following excision of the biopsy, DNA isolated from the tumor is sequenced to identify mutations found in the cancer that are recognized as neoantigens.

In some embodiments, mutated neoantigens are inserted into autologous dendritic cells, which are co-cultured with the tumor infiltrating lymphocytes. Tumor infiltrating lymphocytes are then assayed for neoantigen recognition. Those tumor infiltrating lymphocytes that recognize the neoantigen are then selected, expanded, and transfused back into the subject. In other methods, the T cells are expanded in number due to their capacity to recognize the tumor biopsy from which they were isolated and are infused back into the patient.

In some embodiments, TCR therapy involves engineering a subject's or donor's T cells to express a specific T-cell receptor (“TCR”). The T cell receptor is a heterodimer consisting of two subunits, TCRα and TCRβ. Each subunit contains a constant region that anchors the receptor to the cell membrane and a hypervariable region that functions in antigen recognition. TCRs can recognize tumor specific proteins on the inside and outside of cells.

In some embodiments, in TCR therapy, T cells are harvested from a subject's or donor's blood. The T cells are genetically modified in the laboratory to express a new T cell receptor. The T cells are expanded in number and infused back into the subject. The T cells with the new T cell receptor may target a patient's cancer.

In some embodiments, in chimeric antigen receptor (“CAR”) T cell therapy (“CAR-T therapy”), one or more parts of a T cell receptor is changed into an antigen binding moiety, such as an antibody or antibody fragment. A cancer associated antigen (tumor association antigen or “TAA”) is often expressed by tumors. The antibody or antibody fragment is targeted to the TAA. T cells targeted to a TAA may directly attack cancer cells.

In some embodiments, in CAR-T therapy, T cells are harvested from a subject's or donor's blood and genetically modified to express a chimeric antigen receptor. T cells are expanded in number and infused back into the subject. CAR-T modifications target T cells specifically to the subject's cancer.

In some embodiments, immune checkpoint inhibitors have shown considerable promise in the treatment of diseases, such as cancer. Adoptive cell therapy has likewise shown considerable promise. An inhibitor of a checkpoint, such as a CD3 or a CD28 antibody, and adoptive cell therapy administered in combination may show even greater promise than either of the therapies alone.

The term “adoptive cell therapy” refers to immunotherapy in which immune cells are administered to a subject to help the subject fight diseases, such as cancer or a viral infection. In cancer therapy, for example, T cells are taken from a subject's own blood (or from a donor's blood) or tumor tissue, grown in large numbers, and then given back to the subject to help the subject fight cancer. Types of adoptive cell therapy include tumor-infiltrating lymphocyte (“TIL”) therapy, T-cell receptor (“TCR”) therapy, and chimeric antigen receptor T-cell (CAR-T-cell) therapy. Adoptive cell therapy compositions are known in the art. See, e.g., US 2023/0158072.

The term “tumor-infiltrating lymphocyte (“TIL”) therapy” or “TIL therapy” refers to an immunotherapy that is an adoptive cell therapy that uses lymphocytes that are in or near a tumor and have an ability to recognize the tumor. In TIL therapy, lymphocytes, such as T-cells, that are in or near a tumor are isolated and then treated with substances that makes them grow to large numbers quickly. Those lymphocytes are then given back to the subject.

The term “T-cell receptor therapy” or “TCR therapy” refers to a type of adoptive cell therapy that involves engineering a subject's or donor's T or immune cells to express a particular or specific T-cell receptor or TCR.

The term “Chimeric antigen receptor T cell therapy” or “CAR-T therapy” refers to an adoptive cell therapy where one or more parts of a T cell receptor is changed into an extracellular binding moiety, such as an antibody or antibody fragment. The extracellular binding moiety, such as the antibody or antibody fragment, can be targeted to a tumor associated antigen (TAA) or a tumor specific antigen (TSA).

The term “T cells” or “T lymphocytes” refer to a type of lymphocyte which develops in the thymus and plays a central role in immune response. T cells can be distinguished from other lymphocytes by the presence of a T cell receptor on the cell surface.

The term “TCR complex” refers to the TCR, G-chain, and CD3 molecules together.

The term “T cell receptor” or “TCR” refers to a molecule found on the surface of T cells that is responsible for recognizing fragments of antigen as peptides bound to major histocompatibility (MHC) complex molecules. The T cell receptor is a heterodimer consisting of two subunits, either TCRα and TCRβ or TCRγ and TCRδ. When a TCR engages with an antigen and an MHC, the T cell is activated through signal transduction.

In some embodiments, the CD3 T cell co-receptor helps to activate both the cytotoxic T cell (CD8+ naive T cells) and also T helper cells (CD4+ naive T cells). The CD3 consists of a protein complex. In mammals, the complex contains a CD3γ chain, a CD36 chain, and two CD3ε chains. CD3 chains associate with the T cell receptor and the ζ-chain (zeta-chain) to generate an activation signal.

The term “ζ-chain” or “CD3ζ signaling domain” or “CD3ζ or “zeta-chain” or “T-cell surface recognition glycoprotein CD3 zeta chain” or “CD247” refers to a protein that plays an important role in coupling antigen recognition to intracellular signal transduction pathways.

The term “peripheral blood mononuclear cell” or “PBMCs” refers to any peripheral blood cell having a round nucleus. Peripheral blood cells include, for example, lymphocytes and monocytes.

The term “peripheral blood cell” refers to the cellular components of blood, consisting of red blood cells, such as erythrocytes, white blood cells, such as leucocytes, and platelets. Peripheral blood cells are found within the circulating poll of blood and not sequestered within the lymphatic system, spleen, liver, or bone marrow.

The term “stimulation” or “stimulatory” refers to an event where binding of a molecule (i.e., a stimulatory molecule) mediates signal transduction. A stimulatory molecule is a molecule on an immune cell, such as a T cell, that binds a cognate stimulatory ligand or receptor present on an antigen presenting cell. When present on the antigen presenting cell (e.g., a dendritic cell, a B-cell, and the like), the stimulatory ligand or receptor can specifically bind with a stimulatory ligand or receptor on a T cell, thereby mediating a primary response by the T cell including, but not limited to, activation and initiation of an immune response. Stimulatory molecules include, but are not limited to, an MHC molecule loaded with a peptide, an anti-CD3 antibody, an anti-CD28 antibody, or an anti-CD2 antibody.

The term a “costimulatory signal” refers to a signal which, in combination with a stimulatory signal, leads to a T cell or antigen-presenting cell response such as proliferation, and upregulation or down regulation of an immune response.

The term a “costimulatory molecule” refers to a cognate binding partner on a T cell or antigen-presenting cell that binds with a costimulatory ligand or receptor and mediates a costimulatory response on a T cell or antigen-presenting cell such as proliferation. Costimulatory molecules include, but are not limited to, CD27, CD28, 4-1BB, GITR, OX40, CD30, CD40, CD83, ICOS, LFA-1, CD2, TNFSF14, NKG2C, and CD83.

In some embodiments, the attenuated cancer cells are obtained from a tumor of the individual. In some embodiments, the tumor is a solid tumor. In some embodiments, the tumor is a liquid tumor.

In some embodiments, the attenuated cancer cells are prepared by (a) harvesting cancer cells from a biopsy of a site of tumor from the individual, (b) culturing the harvested cancer cells to a therapeutically relevant amount, and (c) irradiating the cultured cancer cells.

In some embodiments, the immunostimulatory adjuvant comprises a Toll like receptor (TLR) agonist.

In some embodiments, the immunostimulatory adjuvant comprises an anti-CD40 antibody.

In some embodiments, the immune response comprises an adaptive immune response specific to the attenuated cancer cells.

In some embodiments, the present disclosure provides a method of treating cancer in a subject comprising administering a therapeutically effective amount of ESK981 and/or apilimod in combination with a personalized tumor vaccine.

In some embodiments, the personalized tumor vaccines comprises: (a) a phagocytosis stimulating agent, (b) an immunostimulatory adjuvant, and/or (c) attenuated cancer cells.

The term an “attenuated” cell as used herein refers to a cell that is alive but replication deficient. The attenuated cell may be alive but unable to complete its cell-cycle. The attenuated cell may have a limited capacity to replicate, express proteins, and to develop through some life cycle stages, for example, an attenuated cell may be arrested at a particular life cycle stage and is unable to developmentally progress beyond that stage.

The term “attenuated cancer cell” as used herein refers to a cancer cell that is attenuated and with reduced oncogenicity. The attenuated cancer cell may be unable to cause or give rise to a tumor. The attenuated cancer cell may also be unable to metastasize or increase a tumor burden of a subject with the tumor. The attenuated cancer cell may comprise damages in their DNA. The attenuated cancer cell can be obtained by various means, for example, by physical and chemical treatments. The attenuated cancer cell can be obtained by irradiation treatments.

The term “personalized cancer vaccine” as used herein refers to a cancer vaccine that can direct a subject-specific immune response to a tumor of a subject. Such a response may be specific to a specific type of tumor from a specific subject. The personalized tumor vaccine as used herein comprises, e.g., attenuated cancer cells. The personalized tumor vaccine may elicit an adaptive immune response to a tumor or tumor cells of a subject. Personalized tumor vaccines are known in the art. See, e.g., US 2023/0054318. See, e.g., Blass and Ott, Nat Rev Clin Oncol 18: 215-229 (2021).

### Particular Embodiments

The present disclosure provides the following particular embodiments with respect to methods of treating cancer with ESK981 and/or apilimod in combination with an immunotherapy.

Embodiment 1. A method of treating cancer in a subject in need thereof, the method comprising administering to the subject a therapeutically effective amount of ESK981 and/or apilimod, to a subject in combination with a therapeutically effective amount of an immunotherapy.

Embodiment 2. The method of Embodiment 1, wherein the immunotherapy is CD8′ T cell- and major histocompatibility complex (MHC) class I-dependent immunotherapy.

Embodiment 3. The method of Embodiments 1 or 2, wherein the immunotherapy is an immune checkpoint inhibitor, adoptive cell therapy (ACT), or a personalized cancer vaccine.

Embodiment 4. The method of Embodiment 3, wherein the immunotherapy is an immune checkpoint inhibitor.

Embodiment 5. The method of Embodiment 4, wherein the immune checkpoint inhibitor is selected from the group consisting of a PD-1 inhibitor, a PD-L1 inhibitor, a CTLA-4 inhibitor, a LAG3 inhibitor, a TIM3 inhibitor, a cd47 inhibitor a TIGIT inhibitor, and a B7-HI inhibitor.

Embodiment 6. The method of Embodiment 5, wherein the immune checkpoint inhibitor is a PD-1 inhibitor.

Embodiment 7. The method of Embodiment 6, wherein the PD-1 inhibitor is an anti-PD-1 antibody.

Embodiment 8. The method of Embodiment 7, wherein the anti-PD-1 antibody is selected from the group consisting of nivolumab, pembrolizumab, pidilizmab, STI-A1014, STI-A1110, PDR001, MEDI-0680, AGEN2034, BGB-A317, AB122, TSR-042, PF-06801591, cemiplimab, SYM021, JNJ-63723283, HLX10, LZM009, and MGA012.

Embodiment 9. The method of Embodiment 5, wherein the immune checkpoint inhibitor is a PD-L1 inhibitor.

Embodiment 10. The method of Embodiment 9, wherein the PD-L1 inhibitor is an anti-PD-L1 antibody.

Embodiment 11. The method of Embodiment 10, wherein the PD-L1 antibody is selected from the group consisting of avelumab, atezolizumab, durvalumab, STI-A1014, and BMS-936559.

Embodiment 12. The method of Embodiment 5, wherein the immune checkpoint inhibitor is a CTLA-4 inhibitor.

Embodiment 13. The method of Embodiment 12, wherein the CTLA-4 inhibitor is an anti-CTLA-4 antibody.

Embodiment 14. The method of Embodiment 13, wherein the CTLA-4 antibody is selected from the group consisting of ipilimumab and tremelimumab.

Embodiment 15. The method of Embodiment 5, wherein the immune checkpoint inhibitor is a LAG3 inhibitor.

Embodiment 16. The method of Embodiment 15, wherein the LAG3 inhibitor is an anti-LAG3 antibody.

Embodiment 17. The method of Embodiment 16, wherein the anti-LAG3 antibody is GSK2831781.

Embodiment 18. The method of Embodiment 5, wherein the immune checkpoint inhibitor is TIM3 inhibitor.

Embodiment 19. The method of Embodiment 18, wherein the TIM3 inhibitor is an anti-TIM3 antibody.

Embodiment 20. The method of Embodiment 5, wherein the immune checkpoint inhibitor is a cd47 inhibitor.

Embodiment 21. The method of Embodiment 5, wherein the immune checkpoint inhibitor is a TIGIT inhibitor.

Embodiment 22. The method of Embodiment 5, wherein the immune checkpoint inhibitor is a B7-HI inhibitor.

Embodiment 23. The method of any one of Embodiments 1-22, wherein the cancer is or has become resistant to treatment with at least one immune checkpoint inhibitor.

Embodiment 24. The method of any one of Embodiments 1-22, wherein the cancer is a solid tumor.

Embodiment 25 The method of any one of Embodiments 1-22, wherein the cancer is any cancer listed in Table 1 and/or Table 2.

Embodiment 26. The method any one of Embodiments 1-22, wherein the cancer is pancreatic cancer.

Embodiment 27. The method of any one of Embodiments 1-22, wherein the cancer is melanoma.

Embodiment 28. The method of any one of Embodiments 1-22, wherein the cancer is breast cancer.

Embodiment 29. The method of any one of Embodiments 1-22, wherein the cancer is prostate cancer.

Embodiment 30. The method any one of Embodiments 1-22, wherein the cancer is pancreatic ductal adenocarcinoma (PDAC).

Embodiment 31. The method of Embodiment 3, wherein the immunotherapy is adoptive cell therapy.

Embodiment 32. The method of Embodiment 31, wherein the adoptive cell therapy comprises T cells, dendritic cells, macrophages, peripheral blood mononuclear cells (PBMCs), or a combination thereof.

Embodiment 33. The method of Embodiment 32, wherein the adoptive cell therapy comprises T cells.

Embodiment 34. The method of Embodiment 31, wherein the adoptive cell therapy comprises engineered cells expressing a heterologous T cell receptor (TCR), a chimeric antigen receptor (CAR), or a combination thereof.

Embodiment 35. The method of Embodiment 34, wherein the adoptive cell therapy comprises engineered cells expressing a heterologous T cell receptor (TCR).

Embodiment 36. The method of Embodiment 34, wherein the engineered cells express a heterologous T cell receptor (TCR) comprising an extracellular binding moiety that specifically binds a tumor-associated antigen (TAA).

Embodiment 37. The method of Embodiment 35, wherein the heterologous T cell receptor (TCR) comprises an anti-CD3 binding moiety.

Embodiment 38. The method of Embodiment 35, wherein the heterologous T cell receptor (TCR) comprises an anti-CD28 binding moiety.

Embodiment 39. The method of Embodiment 35, wherein the heterologous T cell receptor (TCR) comprises an extracellular binding moiety, a transmembrane domain, and an intracellular domain that triggers the activation, proliferation, or both of lymphocytes.

Embodiment 40. The method of Embodiment 39, wherein the intracellular domain comprises a CD3 signaling domain.

Embodiment 41. The method of Embodiment 39, wherein the intracellular domain comprises a CD28 signaling domain.

Embodiment 42. The method of Embodiment 39, wherein the heterologous T cell receptor (TCR) comprises at least one costimulatory domain.

Embodiment 43. The method of Embodiment 42, wherein the heterologous T cell receptor (TCR) comprises a CD28 costimulatory domain.

Embodiment 44. The method of Embodiment 3, wherein the immunotherapy is a personalized cancer vaccine.

Embodiment 45. The method of Embodiment 44, wherein the personalized cancer vaccine comprises a phagocytosis stimulating agent, an immunostimulatory adjuvant, and attenuated cancer cells, wherein the personalized cancer vaccine, when administered to a subject in need thereof, is effective to activate an immune response.

Embodiment 46. The method of Embodiment 45, wherein the attenuated cancer cells are obtained from a tumor of the subject in need thereof.

Embodiment 47. The method of Embodiment 46, wherein the tumor is selected from the group consisting of a solid tumor or a liquid tumor.

Embodiment 48. The method of Embodiment 45, wherein the immunostimulatory adjuvant comprises a Toll like receptor (TLR) agonist.

Embodiment 49. The method of Embodiment 45, wherein the immunostimulatory adjuvant comprises an anti-CD3 antibody.

Embodiment 50. The method of Embodiment 45, wherein the immunostimulatory adjuvant comprises an anti-CD28 antibody.

Embodiment 51. A method of treating cancer in a subject in need thereof, the method comprising administering a therapeutically effective amount of ESK981 or apilimod to the subject in combination with a therapeutically effective amount of an immunotherapy, wherein the cancer is characterized as having an overexpression of PIKfyve.

Embodiment 52. A method of treating cancer in a subject, the method comprising:
(a) determining whether an overexpression of PIKfyve is present or absent in a biological sample taken from the subject; and
(b) administering a therapeutically effective amount of ESK981 or apilimod in combination with a therapeutically effective amount of an immunotherapy to the subject if an overexpression of PIKfyve is present in the biological sample.

Embodiment 53. A method, comprising administering a therapeutically effective amount of ESK981 or apilimod in combination with a therapeutically effective amount of an immunotherapy to a subject in need thereof, wherein:
(a) the subject has cancer; and
(b) the cancer is characterized as having an overexpression of PIKfyve.

Embodiment 54. A method of identifying whether a subject having cancer as a candidate for treatment with ESK981 or apilimod in combination with a therapeutically effective amount of an immunotherapy, the method comprising:
(a) determining whether an overexpression of PIKfyve is present or absent in a biological sample taken from the subject; and
(b) identifying the subject as being a candidate for treatment if an overexpression of PIKfyve is present; or
(c) identifying the subject as not being a candidate for treatment if an overexpression of PIKfyve is absent.

Embodiment 55. A method of predicting treatment outcome in a subject having cancer, the method comprising determining whether an overexpression of PIKfyve is present or absent in a biological sample taken from the subject, wherein:
(a) the presence of an overexpression of PIKfyve in the biological sample indicates that administering ESK981 or apilimod in combination with a therapeutically effective amount of an immunotherapy to the subject will likely cause a favorable therapeutic response; and
(b) the absence of an overexpression of PIKfyve in the biological sample indicates that administering ESK981 or apilimod in combination with a therapeutically effective amount of an immunotherapy to the subject will likely cause an unfavorable therapeutic response

Embodiment 56. The method of any one of Embodiments 1-55, wherein the cancer is any cancer listed in Table 1 and/or Table 2.

Embodiment 57. The method of any one of claims 1-56, wherein the cancer is or has become resistant to immunotherapy in the absence of ESK981 or apilimod.

Embodiment 58. The method of any one of claims 1-56, wherein a therapeutically effective amount of ESK981 is administered to the subject in combination with a therapeutically effective amount of an immunotherapy.

Embodiment 59. The method of any one of claims 1-56, wherein a therapeutically effective amount of apilimod is administered to the subject in combination with a therapeutically effective amount of an immunotherapy.

Embodiment 60. The method of any one of claims 51-59, wherein the immunotherapy is ACT.

Embodiment 61. The method of any one of claims 51-59, wherein the immunotherapy is a personalized cancer vaccine.

The present diclsoure provides the following particular embodiments with respect to ESK981 and/or apilimod for use to treat cancer in combination with an immunotherapy.

Embodiment 1. Use of ESK981 and/or apilimod for treating cancer in subject in need thereof, wherein the ESK981 and/or apilimod is to be administered in combination with a therapeutically effective amount of immunotherapy.

Embodiment 2. The use of Embodiment 1, wherein the immunotherapy is CD8⁺ T cell- and major histocompatibility complex (MHC) class I-dependent immunotherapy.

Embodiment 3. The use of Embodiments 1 or 2, wherein the immunotherapy is an immune checkpoint inhibitor, adoptive cell therapy (ACT), or a personalized cancer vaccine.

Embodiment 4. The use of Embodiment 3, wherein the immunotherapy is an immune checkpoint inhibitor.

Embodiment 5. The use of Embodiment 4, wherein the immune checkpoint inhibitor is selected from the group consisting of a PD-1 inhibitor, a PD-L1 inhibitor, a CTLA-4 inhibitor, a LAG3 inhibitor, a TIM3 inhibitor, a cd47 inhibitor a TIGIT inhibitor, and a B7-HI inhibitor.

Embodiment 6. The use of Embodiment 5, wherein the immune checkpoint inhibitor is a PD-1 inhibitor.

Embodiment 7. The use of Embodiment 6, wherein the PD-1 inhibitor is an anti-PD-1 antibody.

Embodiment 8. The use of Embodiment 7, wherein the anti-PD-1 antibody is selected from the group consisting of nivolumab, pembrolizumab, pidilizmab, STI-A1014, STI-A1110, PDR001, MEDI-0680, AGEN2034, BGB-A317, AB122, TSR-042, PF-06801591, cemiplimab, SYM021, JNJ-63723283, HLX10, LZM009, and MGA012.

Embodiment 9. The use of Embodiment 5, wherein the immune checkpoint inhibitor is a PD-L1 inhibitor.

Embodiment 10. The use of Embodiment 9, wherein the PD-L1 inhibitor is an anti-PD-L1 antibody.

Embodiment 11. The use of Embodiment 10, wherein the PD-L1 antibody is selected from the group consisting of avelumab, atezolizumab, durvalumab, STI-A1014, and BMS-936559.

Embodiment 12. The use of Embodiment 5, wherein the immune checkpoint inhibitor is a CTLA-4 inhibitor.

Embodiment 13. The use of Embodiment 12, wherein the CTLA-4 inhibitor is an anti-CTLA-4 antibody.

Embodiment 14. The use of Embodiment 13, wherein the CTLA-4 antibody is selected from the group consisting of ipilimumab and tremelimumab.

Embodiment 15. The use of Embodiment 5, wherein the immune checkpoint inhibitor is a LAG3 inhibitor.

Embodiment 16. The use of Embodiment 15, wherein the LAG3 inhibitor is an anti-LAG3 antibody.

Embodiment 17. The use of Embodiment 16, wherein the anti-LAG3 antibody is GSK2831781.

Embodiment 18. The use of Embodiment 5, wherein the immune checkpoint inhibitor is TIM3 inhibitor.

Embodiment 19. The use of Embodiment 18, wherein the TIM3 inhibitor is an anti-TIM3 antibody.

Embodiment 20. The use of Embodiment 5, wherein the immune checkpoint inhibitor is a cd47 inhibitor.

Embodiment 21. The use of Embodiment 5, wherein the immune checkpoint inhibitor is a TIGIT inhibitor.

Embodiment 22. The use of Embodiment 5, wherein the immune checkpoint inhibitor is a B7-HI inhibitor.

Embodiment 23. The use of any one of Embodiments 1-22, wherein the cancer is or has become resistant to treatment with at least one immune checkpoint inhibitor.

Embodiment 24. The use of any one of Embodiments 1-22, wherein the cancer is a solid tumor.

Embodiment 25 The use of any one of Embodiments 1-22, wherein the cancer is any cancer listed in Table 1 and/or Table 2.

Embodiment 26. The use any one of Embodiments 1-22, wherein the cancer is pancreatic cancer.

Embodiment 27. The use of any one of Embodiments 1-22, wherein the cancer is melanoma.

Embodiment 28. The use of any one of Embodiments 1-22, wherein the cancer is breast cancer.

Embodiment 29. The use of any one of Embodiments 1-22, wherein the cancer is prostate cancer.

Embodiment 30. The use any one of Embodiments 1-22, wherein the cancer is pancreatic ductal adenocarcinoma (PDAC).

Embodiment 31. The use of Embodiment 3, wherein the immunotherapy is an adoptive cell therapy.

Embodiment 32. The use of Embodiment 31, wherein the adoptive cell therapy comprises T cells, dendritic cells, macrophages, peripherial blood mononuclear cells (PBMCs), or a combination thereof.

Embodiment 33. The use of Embodiment 32, wherein the adoptive cell therapy comprises T cells.

Embodiment 34. The use of Embodiment 31, wherein the adoptive cell therapy comprises engineered cells expressing a heterologous T cell receptor (TCR), a chimeric antigen receptor (CAR), or a combination thereof.

Embodiment 35. The use of Embodiment 34, wherein the adoptive cell therapy comprises engineered cells expressing a heterologous T cell receptor (TCR).

Embodiment 36. The use of Embodiment 34, wherein the engineered cells express a heterologous T cell receptor (TCR) comprising an extracellular binding moiety that specifically binds a tumor-associated antigen (TAA).

Embodiment 37. The use of Embodiment 35, wherein the heterologous T cell receptor (TCR) comprises an anti-CD3 binding moiety.

Embodiment 38. The use of Embodiment 35, wherein the heterologous T cell receptor (TCR) comprises an anti-CD28 binding moiety.

Embodiment 39. The use of Embodiment 35, wherein the heterologous T cell receptor (TCR) comprises an extracellular binding moiety, a transmembrane domain, and an intracellular domain that triggers the activation, proliferation, or both of lymphocytes.

Embodiment 40. The use of Embodiment 39, wherein the intracellular domain comprises a CD3 signaling domain.

Embodiment 41. The use of Embodiment 39, wherein the intracellular domain comprises a CD28 signaling domain.

Embodiment 42. The use of Embodiment 39, wherein the heterologous T cell receptor (TCR) comprises at least one costimulatory domain.

Embodiment 43. The use of Embodiment 42, wherein the heterologous T cell receptor (TCR) comprises a CD28 costimulatory domain.

Embodiment 44. The use of Embodiment 3, wherein the immunotherapy is a personalized cancer vaccine.

Embodiment 45. The use of Embodiment 44, wherein the personalized cancer vaccine comprises a phagocytosis stimulating agent, an immunostimulatory adjuvant, and attenuated cancer cells, wherein the personalized cancer vaccine, when administered to a subject in need thereof, is effective to activate an immune response.

Embodiment 46. The use of Embodiment 45, wherein the attenuated cancer cells are obtained from a tumor of the subject in need thereof.

Embodiment 47. The use of Embodiment 46, wherein the tumor is selected from the group consisting of a solid tumor or a liquid tumor.

Embodiment 48. The use of Embodiment 46, wherein the tumor is derived from a solid tumor.

Embodiment 49 The use of Embodiment 45, wherein the immunostimulatory adjuvant comprises a Toll like receptor (TLR) agonist.

Embodiment 50. The use of Embodiment 45, wherein the immunostimulatory adjuvant comprises an anti-CD3 antibody.

Embodiment 51. The use of Embodiment 45, wherein the immunostimulatory adjuvant comprises an anti-CD28 antibody.

The present diclsoure provides the following particular embodiments with respect to the use of ESK981 and/or apilimod in the manufacture of a medicament for treating cancer in combination with an immunotherapy.

Embodiment 1. Use of ESK981 and/or apilimod in the manufacture treating cancer in subject in need thereof, wherein the ESK981 and/or apilimod is to be administered in combination with a therapeutically effective amount of immunotherapy.

Embodiment 2. The use of Embodiment 1, wherein the immunotherapy is CD8⁺ T cell- and major histocompatibility complex (MHC) class I-dependent immunotherapy.

Embodiment 3. The use of Embodiments 1 or 2, wherein the immunotherapy is an immune checkpoint inhibitor, adoptive cell therapy (ACT), or a personalized cancer vaccine.

Embodiment 4. The use of Embodiment 3, wherein the immunotherapy is an immune checkpoint inhibitor.

Embodiment 5. The use of Embodiment 4, wherein the immune checkpoint inhibitor is selected from the group consisting of a PD-1 inhibitor, a PD-L1 inhibitor, a CTLA-4 inhibitor, a LAG3 inhibitor, a TIM3 inhibitor, a cd47 inhibitor a TIGIT inhibitor, and a B7-HI inhibitor.

Embodiment 6. The use of Embodiment 5, wherein the immune checkpoint inhibitor is a PD-1 inhibitor.

Embodiment 7. The use of Embodiment 6, wherein the PD-1 inhibitor is an anti-PD-1 antibody.

Embodiment 8. The use of Embodiment 7, wherein the anti-PD-1 antibody is selected from the group consisting of nivolumab, pembrolizumab, pidilizmab, STI-A1014, STI-A1110, PDR001, MEDI-0680, AGEN2034, BGB-A317, AB122, TSR-042, PF-06801591, cemiplimab, SYM021, JNJ-63723283, HLX10, LZM009, and MGA012.

Embodiment 9. The use of Embodiment 5, wherein the immune checkpoint inhibitor is a PD-L1 inhibitor.

Embodiment 10. The use of Embodiment 9, wherein the PD-L1 inhibitor is an anti-PD-L1 antibody.

Embodiment 11. The use of Embodiment 10, wherein the PD-L1 antibody is selected from the group consisting of avelumab, atezolizumab, durvalumab, STI-A1014, and BMS-936559.

Embodiment 12. The use of Embodiment 5, wherein the immune checkpoint inhibitor is a CTLA-4 inhibitor.

Embodiment 13. The use of Embodiment 12, wherein the CTLA-4 inhibitor is an anti-CTLA-4 antibody.

Embodiment 14. The use of Embodiment 13, wherein the CTLA-4 antibody is selected from the group consisting of ipilimumab and tremelimumab.

Embodiment 15. The use of Embodiment 5, wherein the immune checkpoint inhibitor is a LAG3 inhibitor.

Embodiment 16. The use of Embodiment 15, wherein the LAG3 inhibitor is an anti-LAG3 antibody.

Embodiment 17. The use of Embodiment 16, wherein the anti-LAG3 antibody is GSK2831781.

Embodiment 18. The use of Embodiment 5, wherein the immune checkpoint inhibitor is TIM3 inhibitor.

Embodiment 19. The use of Embodiment 18, wherein the TIM3 inhibitor is an anti-TIM3 antibody.

Embodiment 20. The use of Embodiment 5, wherein the immune checkpoint inhibitor is a cd47 inhibitor.

Embodiment 21. The use of Embodiment 5, wherein the immune checkpoint inhibitor is a TIGIT inhibitor.

Embodiment 22. The use of Embodiment 5, wherein the immune checkpoint inhibitor is a B7-HI inhibitor.

Embodiment 23. The use of any one of Embodiments 1-22, wherein the cancer is or has become resistant to treatment with at least one immune checkpoint inhibitor.

Embodiment 24. The use of any one of Embodiments 1-22, wherein the cancer is a solid tumor.

Embodiment 25 The use of any one of Embodiments 1-22, wherein the cancer is any cancer listed in Table 1 and/or Table 2.

Embodiment 26. The use any one of Embodiments 1-22, wherein the cancer is pancreatic cancer.

Embodiment 27. The use of any one of Embodiments 1-22, wherein the cancer is melanoma.

Embodiment 28. The use of any one of Embodiments 1-22, wherein the cancer is breast cancer.

Embodiment 29. The use of any one of Embodiments 1-22, wherein the cancer is prostate cancer.

Embodiment 30. The use any one of Embodiments 1-22, wherein the cancer is pancreatic ductal adenocarcinoma (PDAC).

Embodiment 31. The use of Embodiment 3, wherein the immunotherapy is an adoptive cell therapy.

Embodiment 32. The use of Embodiment 31, wherein the adoptive cell therapy comprises T cells, dendritic cells, macrophages, peripherial blood mononuclear cells (PBMCs), or a combination thereof.

Embodiment 33. The use of Embodiment 32, wherein the adoptive cell therapy comprises T cells.

Embodiment 34. The use of Embodiment 31, wherein the adoptive cell therapy comprises engineered cells expressing a heterologous T cell receptor (TCR), a chimeric antigen receptor (CAR), or a combination thereof.

Embodiment 35. The use of Embodiment 34, wherein the adoptive cell therapy comprises engineered cells expressing a heterologous T cell receptor (TCR).

Embodiment 36. The use of Embodiment 34, wherein the engineered cells express a heterologous T cell receptor (TCR) comprising an extracellular binding moiety that specifically binds a tumor-associated antigen (TAA).

Embodiment 37. The use of Embodiment 35, wherein the heterologous T cell receptor (TCR) comprises an anti-CD3 binding moiety.

Embodiment 38. The use of Embodiment 35, wherein the heterologous T cell receptor (TCR) comprises an anti-CD28 binding moiety.

Embodiment 39. The use of Embodiment 35, wherein the heterologous T cell receptor (TCR) comprises an extracellular binding moiety, a transmembrane domain, and an intracellular domain that triggers the activation, proliferation, or both of lymphocytes.

Embodiment 40. The use of Embodiment 39, wherein the intracellular domain comprises a CD3 signaling domain.

Embodiment 41. The use of Embodiment 39, wherein the intracellular domain comprises a CD28 signaling domain.

Embodiment 42. The use of Embodiment 39, wherein the heterologous T cell receptor (TCR) comprises at least one costimulatory domain.

Embodiment 43. The use of Embodiment 42, wherein the heterologous T cell receptor (TCR) comprises a CD28 costimulatory domain.

Embodiment 44. The use of Embodiment 3, wherein the immunotherapy is a personalized cancer vaccine.

Embodiment 45. The use of Embodiment 44, wherein the personalized cancer vaccine comprises a phagocytosis stimulating agent, an immunostimulatory adjuvant, and attenuated cancer cells, wherein the personalized cancer vaccine, when administered to a subject in need thereof, is effective to activate an immune response.

Embodiment 46. The use of Embodiment 45, wherein the attenuated cancer cells are obtained from a tumor of the subject in need thereof.

Embodiment 47. The use of Embodiment 46, wherein the tumor is selected from the group consisting of a solid tumor or a liquid tumor.

Embodiment 48. The use of Embodiment 46, wherein the tumor is derived from solid tumor cells.

Embodiment 49 The use of Embodiment 45, wherein the immunostimulatory adjuvant comprises a Toll like receptor (TLR) agonist.

Embodiment 50. The use of Embodiment 45, wherein the immunostimulatory adjuvant comprises an anti-CD3 antibody.

Embodiment 51. The use of Embodiment 45, wherein the immunostimulatory adjuvant comprises an anti-CD28 antibody.

### EXAMPLES

The following examples are illustrative, but not limiting, of the compounds, compositions, methods, and use of the present disclosure. Other suitable modifications and adaptations of the variety of conditions and parameters normally encountered in clinical therapy and which are obvious to those skilled in the art are within the spirit and scope of the present disclosure.

### General Methods and Materials

### Cell Lines

The KPC1361 cell line was generated from a pancreatic tumor of a genetically engineered mouse model (LSL-Kras^{G12D/+}; LSL-Trp53^{R172H/+}; Pdx1-Cre). Briefly, the tumor was cut into small pieces with surgical scissors and digested with collagenase D (Roche; catalog #: COLLD-RO) at 0.5 mg/ml and DNase I (Roche; catalog #: 10104159001) at 0.25 mg/ml, at 37° C. for 45 minutes. The suspension was then filtered with a 70 μm cell strainer, and tumor cells were enriched and maintained in DMEM, high glucose (Gibco; catalog #: 10566016) supplemented with 10% (v/v) fetal bovine serum (Gibco; catalog #: 16140071) and 50 U/ml penicillin-streptomycin (Gibco; catalog #: 15140-122). Cells used in the study were from passage 8-30. B16-F10, B16-BL6, 4T1, MyC-CaP, MIA PaCa2, and LNCaP cell lines were acquired from American Type Culture Collection (ATCC, Manassas, Virginia), and authenticated regularly with sequencing by Labcorp Cell Line Testing division (Burlington, North Carolina). All cell lines were maintained at 37° C. in 5% carbon dioxide and 95% atmospheric air. Mycoplasma test was performed every two weeks to ensure that all cells used in the study were mycoplasma-free.

### Stable Cell Lines with Gene Knockout or Overexpression

Single guide RNAs (sgRNAs) designed to target constitutive exons nearest to the start codon of the target genes were subjected to off-target prediction with Off-Spotter, and sgRNAs with the lowest off-target potential were used. The list of sgRNAs used in this study can be found in Table 3. The sgRNAs were then constructed into lentiCRISPR v2 (Addgene; #52961) with Golden Gate reaction. Briefly, the annealed oligos were mixed with the backbone plasmid in a reaction containing BsmBI and T7 ligase. Following 15 cycles of digestion and ligation, the product from the reaction was used to transform Stbl3 competent cells. Successful insertion of the oligos was confirmed with Sanger sequencing by Eurofins Genomics (Louisville, Kentucky). To generate cells with the gene knockout, the vector containing the sgRNA was transiently transfected into target cells with Lipofectamine 3000 (Thermo Fisher Scientific; catalog #: L3000001) according to the manufacturer's instructions. After puromycin selection, single cells were seeded to 96 well dishes. Colonies from the single cells were then expanded to generate the sublines with the gene knockout. Depletion of the target gene was confirmed by western blot and Sanger sequencing.

TABLE 3

| | | SEQ |
|---|---|---|
| sgRNA | Target Sequence | ID NO: |
| sgPikfyve #1 | CTAACACTGAAGAGCGCCGG | 1 |
| sgPikfyve #2 | TGACAAGAGTTCCCCGACAC | 2 |
| sgAtg5 #1 | GGCCATCAACCGGAAACTCA | 3 |
| sgAtg5 #2 | TCCATCCAAGGATGCGGTTG | 4 |
| sgAtg7 | GAAGTTGAACGAGTACCGCC | 5 |
| sgB2m #1 | ATTTGGATTTCAATGTGAGG | 6 |
| sgB2m #2 | AGTATACTCACGCCACCCAC | 7 |
| Nontargeting | ACGTGTAAGGCGAACGCCTT | 8 |
| control | | |

To generate the ovalbumin-expressing (OVA) cells, cytoplasmic ovalbumin was amplified from pBabe_Hyg_cOVA_T2A_mStrawberry (Addgene; #161737), with PrimeSTAR® Max DNA Polymerase (Takara Bio; catalog #: R045A) and constructed into pLenti CMVie-IRES-BlastR (Addgene; #119863) See, e.g., Yamamoto, K. et al., Nature 2020; 581(7806):100-105. The constructed vector was next transfected into HEK293T together with pRSV-REV (Addgene; catalog #: 12253), pMDLg/pRRE (Addgene; catalog #: 12251), and pMD2.G (Addgene; catalog #: 12259). Medium containing the virus was collected 72-hours post transfection and filtered with 0.22 μm filter for cell debris removal. Target cells were then transduced with the virus in combination with 4 μg/ml of polybrene (Sigma-Aldrich; catalog #: H9268). One day post viral transduction, the cells were selected with blasticidin S (Thermo Fisher Scientific; catalog #: A1113903) at 4 μg/ml for KPC1361 and 10 μg/ml for B16-F10. The selected cells were used for further experiments.

GFP-labeled cancer cells were generated by transducing virus Lenti-GFP containing CMV-driven GFP to the target cells. The virus was acquired from the Biomedical Research Core Facilities at the University of Michigan (U-M). Two days after the viral transduction, the GFP-positive cells were sorted with a cell sorter (SONY SH800S).

All stable cell lines were tested every two weeks for mycoplasma contamination to ensure that all cells used in the experiments were mycoplasma-free.

### Animal Studies

All experimental protocols were conducted after approval by the U-M Institutional Animal Care & Use Committee. As previous investigations did not show a difference between sexes in ESK981 efficacy, female C57BL/6 mice aged 6-8-week-old were used in the study. KPC1361 cells were resuspended in PBS, 1:1 (v/v) mixed with Matrigel (BD Biosciences; catalog #: 354248), and orthotopically injected to the pancreases at 250,000 cells in a total volume of 50 μl per injection. B16-F10 cells were resuspended in PBS and injected subcutaneously to both flanks at 400,000 cells per injection. For ACT study, B16-F10-OVA cells were resuspended in PBS and injected subcutaneously to both flanks at 300,000 cells per injection. For lung metastasis experiment, B16-BL6 cells were resuspended in PBS, 1:1 (v/v) mixed with Matrigel and injected subcutaneously to both flanks at 400,000 cells per injection. Fifteen days after tumor cell inoculation, the primary tumors were removed surgically under anesthesia. Thirty-five days after removal of the primary tumors, lung tissues were extracted and fixed, and lung metastasis was quantified. C57BL/6 mice were acquired from The Jackson Laboratory. Measurement of tumor volume began 5-9 days post tumor cell implantation and was performed 2-3 times per week using calipers. Volume measurements of pancreatic tumors were also achieved with calipers, as reported See, e.g., Koikawa, K. et al., Cell 2021; 184(18):4753-4771. In brief, mice were subjected to anesthesia with 2-3% isoflurane, and the abdominal wall then became soft and loose. The mouse was next held with the left hand allowing for the palpation and assessment of the firm pancreatic tumor mass with the right hand. Mice with KPC1361 pancreatic tumors did not develop ascites. Volume (mm³) of tumor was calculated using (W²×L)/2, where W stood for minor tumor axis and L for the major. All mice were maintained in a pathogen-free condition, with a 12-hour light/dark cycle.

### In Vivo Treatments

For ICB treatment in KPC1361 pancreatic tumor-bearing mice, randomization was performed when tumors reached approximately 100 mm³. For ACT and vaccine experiments, the mice were randomized when tumors reached approximately 35 mm³. Anti-mouse PD-1 (clone RMP1-14) and its isotype control were purchased from BioXcell, administered intraperitoneally at 250 μg per mouse, biweekly. Apilimod (MedChemExpress; catalog #: HY-14644) and ESK981, acquired from Esanik Therapeutics, were suspended in Suspending Vehicle Ora-Plus (Perrigo) and administered via oral gavage at 60 and 30 mg/kg, respectively. For ACT, CD8⁺ T cells were isolated from the spleens and lymph nodes of OT1 mice (The Jackson Laboratory; #003831) with Mouse CD8⁺ T Cell Isolation Kit (Stemcell; catalog #: 19853). The cells were then activated and expanded with the mouse T Cell Activation/Expansion kit (Miltenyibiotec; #130-093-627) in T cell-medium containing RPMI 1640 (Gibco; catalog #: 11875093) supplemented with 10 mM HEPES, 10% (v/v) fetal bovine serum (Gibco; catalog #: 16140071), 50 U/ml penicillin-streptomycin (Gibco; catalog #: 15140-122), 27.5 μM beta-mercaptoethanol (Sigma; catalog #: M3148-100ML), and 10 ng/ml mouse recombinant IL-2 (STEMCELL; catalog #: 78081.1), for four days, with medium and activation beads refreshed on day two. The expanded T cells were collected and washed with PBS. The T cells in PBS were next intravenously injected to the animals, on day seven and day 17 post tumor cell inoculation. For vaccine therapy, B16-F10 cells were resuspended in HBSS (HyClone) and irradiated at 35 Gy, and subcutaneously injected with poly(I:C) at two-million irradiated cancer cells plus 30 μg poly(I:C) (Invitrogen; catalog #: vac-pic) per mouse, on day five and day 12 post tumor cell inoculation. For CD8⁺ T cells depletion, anti-mouse CD8α (clone 2.43), and its isotype controls were purchased from BioXcell, and administrated intraperitoneally on days 2, 5, 8, 11, and 14 post tumor cell inoculation, at 400 μg per mouse as a loading dose and 100 μg per mouse subsequently. Efficacy of CD8⁺ T cell depletion was assessed by flow cytometry with blood isolated from the treated mice. No blinding was used in the study.

### Prostate Patient-Derived Xenograft (PDX) Model

The PDX model was acquired as described previously See, e.g., Palanisamy, N. et al., Clin. Cancer Res. 2020; 26(18):4933-4946. Briefly, the model was acquired from a man diagnosed with castration-resistant prostate cancer undertaking cystoprostatectomy. Mixed prostatic adenocarcinoma and neuroendocrine carcinoma was identified by histopathology on the cystoprostatectomy specimen. The tumor was cut into fragments with a 2-mm³ size, coated with 100% Matrigel, and implanted to both flanks in a male severe combined immunodeficiency (SCID) mouse. Tumors formed in mice were expanded and maintained in male SCID mice. For apilimod or ESK981 treatment, the tumor-bearing mice were randomized when the tumors approached 100-200 mm³ in size. Apilimod or ESK981 was administrated as described above.

### Immunocytochemistry

Cancer cells were seeded on cover glasses coated with 0.01% poly-L-Lysine (Sigma-Aldrich; catalog #: A-005-C). The cells were then treated with DMSO, apilimod, or ESK981, and stimulated with 10 ng/ml mouse recombinant IFN-γ (R&D Systems; catalog #: 485-MI) for 24 hours, followed by fixation with 2% paraformaldehyde in PBS and one washing with PBS. The cells were then permeabilized with 0.25% Triton X-100 in PBS, washed with PBS three times, and blocked with 5% goat serum in PBS for one hour at room temperature. After blocking, the cells were incubated with the MHC-I antibody (ER-HR52; Novus Biologicals; catalog #: NB100-64952) at 4° C. overnight, followed by PBS wash for three times and incubation of secondary antibody, Alexa Fluor 488 goat anti-rat IgG antibody (Jackson Immunoresearch; #112-545-167) at room temperature for one hour. After further washing with PBS three times, the cells were stained with DAPI and mounted on slides for imaging.

### Coculture of Cancer Cells and CD8+ T Cells

CD8⁺ T cells were isolated from OT1 mice and activated for four days in vitro as described above. The OVA-expressing cancer cells were established as described above. Cancer cells with Pikfyve knockout were directly cocultured with the activated CD8⁺ T cells, while the apilimod- or ESK981-treated cells were treated with the agent at the indicated concentrations for 24 hours prior to the coculture. To establish the coculture, the cancer cells were seeded into 96 well dishes at 5,000 cells per well, and then 5,000 activated CD8⁺ T cells were added into the well. Two days after the coculture, the CD8⁺ T cells were collected for flow cytometry analysis. After removal of the CD8⁺ T cells, the viable cancer cells were measured by MTT (3-(4,5-Dimethylthiazol-2-yl)-2,5-Diphenyltetrazolium Bromide); Thermo Fisher Scientific; catalog #M6494), according to the instructions from the manufacturer. For models derived from B16-F10, the coculture was performed in the above mentioned T cell-medium. For models derived from KPC1361, the coculture was performed in 45% DMEM, high glucose (Gibco; catalog #: 10566016), 45% DMME RPMI 1640 (Gibco; catalog #: 11875093) supplemented with 10% (v/v) fetal bovine serum (Gibco; catalog #: 16140071), 10 mM HEPES, 50 U/ml penicillin-streptomycin (Gibco; catalog #: 15140-122), 27.5 μM beta-mercaptoethanol (Sigma; catalog #: M3148-100ML), and 10 ng/ml mouse recombinant IL-2 (STEMCELL; catalog #: 78081.1).

### Flow Cytometry Analysis

Cancer cells or the OVA-expressing cancer cells treated in vitro were harvested from dishes, resuspended in buffer MACS (PBS containing 2% FBS and 2 mM EDTA), and stained with Zombie NIR™ Fixable Viability kit (BioLegend: #423106) and fluorophore-conjugated antibody against MHC-I or PE-conjugated antibody against SIINFEKL bound H-2Kb (BioLegend; catalog #141604), respectively. The cells were next washed twice with 1 ml MACS and fixed with 2% paraformaldehyde in PBS for 15 minutes at room temperature. The cells were then subjected to the flow cytometer (SONY SH800S) for measuring surface expression of MHC-I or SIINFEKL bound H-2Kb, respectively. Antibodies used in this study include anti-H-2Kb (BD Biosciences; catalog #553570) and anti-H-2Db (28-14-8; Thermo Fisher Scientific; catalog #12-5999-82) for KPC1361 and B16-F10, anti-H-2Kd (BD Biosciences; catalog #562004) and anti-H-2Dd (BD Biosciences; catalog #553580) for 4T1, anti-H-2Kq (BD Biosciences; catalog #742296) and anti-H-2Dq/H-2Lq (BD Biosciences; catalog #744853) for MyC-CaP, and anti-HLA-A,B,C (clone w6/32; BioLegend; catalog #311406) for human cancer cells.

For measuring the proliferation and functionality of CD8⁺ T cells in the coculture of cancer cells and OT1 CD8⁺ T cells, the T cells were collected from the coculture and stimulated in the above mentioned T cell-medium supplemented with a stimulation cocktail containing 200 ng/ml PMA (Phorbol 12-Myristate 13-Acetate; Sigma; catalog #P1585), 1 μg/ml ionomycin (Sigma; catalog #IO634), 1×brefeldin A (Thermo Fisher Scientific; catalog #00-4506-51) and 1×monensin (Thermo Fisher Scientific; catalog #00-4505-51) at 37° C. for four hours. The cells were then collected, resuspended in MACS, and stained with Zombie Green™ Fixable Viability kit (BioLegend; catalog #423111) and anti-CD8 antibody (BD Biosciences; catalog #560776). After washing with MACS, the cells were next fixed/permeabilized, washed with the Foxp3/Transcription Factor Staining Buffer Set (Thermo Fisher Scientific; catalog #00-5523-00), and stained with anti-Ki67 (Thermo Fisher Scientific; catalog #56-5698-82) and anti-IFN-γ (BD Biosciences; catalog #563773). After further washing with MACS, the cells were subjected to flow cytometry assessment on the BD LSRFortessa™ Cell Analyzer.

For profiling T cells in tumors or tumor-draining lymph nodes, the tissues were cut into small pieces with a blade and digested with collagenase D (Roche; catalog #: COLLD-RO) plus DNase I (Roche; catalog #: 10104159001) at 0.5 and 0.25 mg/ml, respectively, at 37° C. for 40 minutes with agitation. Following filtering with 70 μm cell strainers, the suspensions were carefully layered into centrifuge tubes containing density gradient medium (Lymphoprep; STEMCELL; catalog #07851). After centrifuge, the mononuclear cell layer at the interface was collected and washed once with MACS. The cells were then stimulated with the above-mentioned cocktail containing PMA (Phorbol 12-Myristate 13-Acetate), ionomycin, brefeldin A, and monensin in the T cell-medium at 37° C. for four hours. The cells were next washed once with MACS and blocked with anti-mouse CD16/32 (Biolegend; catalog #: 156604) at room temperature for 5 minutes and stained with anti-CD45 (BD Biosciences; catalog #550994), anti-CD3 (BD Biosciences; catalog #555274), anti-CD90 (BioLegend; catalog #140327), and anti-CD8 (BD Biosciences; catalog #560776), at room temperature for 15 minutes. After staining, the cells were washed once with MACS, and fixed/permeabilized and washed with the Foxp3/Transcription Factor Staining Buffer Set (Thermo Fisher Scientific; catalog #00-5523-00). The cells next were stained for anti-Ki67 (Thermo Fisher Scientific; catalog #56-5698-82), anti-TNF-α (BioLegend; catalog #506324), and anti-IFN-γ (BD Biosciences; catalog #563773) at room temperature for 15 minutes and washed twice with MACS. Absolute Counting Beads (Thermo Fisher Scientific; catalog #: C36950) were then added to the samples for quantification of the target cells according to the instructions from the manufacturer, and the samples were next subjected to flow cytometry assessment on the BD LSRFortessa™ Cell Analyzer.

For TRP2 tetramer staining, the tumor tissues were cut, digested, and filtered as described above. The suspensions were then stained with the Zombie NIR™ Fixable Viability kit (BioLegend: #423106) and Tetramer/BV421 H-2Kb TRP2 (SVYDFFVWL; MBL International; #TB-5004-4) at room temperature for 10 minutes and blocked with anti-mouse CD16/32 (Biolegend; catalog #: 156604) at room temperature for 5 minutes. After blocking, the suspensions were stained with anti-CD45 (BD Biosciences; catalog #550994) and anti-CD8 (BD Biosciences; catalog #560776) for 15 minutes at room temperature. Red blood cells were then lysed with the RBC Lysis Buffer (BioLegend; catalog #: 420301) at room temperature for four minutes, and the cells were then washed twice with MACS and fixed in 0.5% paraformaldehyde in PBS at 4° C. for one hour. Absolute Counting Beads (Thermo Fisher Scientific; catalog #C36950) were next added to the samples for quantification of the target cells according to the instructions from the manufacturer, and then the samples were subjected to flow cytometry assessment on the BD LSRFortessa™ Cell Analyzer.

For measuring MHC-I surface expression in tumor cells, tumors derived from GFP-labeled cancer cells were cut, digested, and filtered as described above. The suspensions were then stained with the Zombie NIR™ Fixable Viability kit (BioLegend: catalog #: 423106) and anti-H-2Kb (BD Biosciences; catalog #553570) plus anti-H-2Db (28-14-8; Thermo Fisher Scientific; catalog #: 12-5999-82) as described above. The suspensions were next washed and fixed as described above and subjected to BD LSRFortessa™ Cell Analyzer for MHC-I measurement.

For measuring CD8⁺ T cells post anti-CD8α antibody treatment, 100 μl of blood was collected from the tail into a tube containing 40 μl 0.5 M EDTA solution (pH 8.0). The suspension was blocked with anti-mouse CD16/32 (Biolegend; cat. no. 156604) as described above and stained with anti-CD45 (BD Biosciences; catalog #: 550994), anti-CD3 (BD Biosciences; catalog #: 555274), anti-CD90 (BioLegend; catalog #: 140327), anti-CD8 (BD Biosciences; catalog #: 560776), and anti-CD4 (BD Biosciences; catalog #: 553051), for 15 minutes at room temperature. Red blood cells were then lysed with the RBC Lysis Buffer (BioLegend; #420301), followed by washing with MACS twice. The samples were then fixed with 2% paraformaldehyde in PBS for 15 minutes, followed by assessment on the BD LSRFortessa™ Cell Analyzer.

All flow cytometry data were analysed with FlowJo V10.8.1.

### Immunofluorescence

Dissected tumor tissues were frozen in TissueTek OCT Compound (Sakura Finetek) by liquid nitrogen. The frozen sample blocks were cut into 5 μm sections and then fixed at room temperature with 2% paraformaldehyde for 15 mins. Permeabilization was then performed on the sections with 0.25% Triton X-100 for 15 mins. After washing with PBS three times, the sections were next blocked with 5% goat serum and incubated with the MHC-I antibody (ER-HR52; Novus Biologicals; catalog #: NB100-64952) at 4° C. overnight, followed by three PBS washes and incubation of secondary antibody, Alexa Fluor 488 goat anti-rat IgG antibody (Jackson Immunoresearch; #112-545-167) for one hour at room temperature. After washing with PBS three times, the sections were stained with DAPI and mounted on slides for imaging.

### Reverse Transcription-Quantitative PCR (RT-qPCR)

Cells were lysed with QIAzol lysis reagent, and RNAs were extracted with the RNeasy Mini kit (Qiagen), according to the instructions from the manufacturer. The RNAs were next converted into cDNA using the Maxima First Strand cDNA Synthesis kit (Thermo Fisher Scientific; catalog #: K1671), according to the user manual. Quantitative PCR was next conducted with the Fast SYBR Green Master Mix (Thermo Fisher Scientific; catalog #: 4385612) on QuantStudio 5 or 7 Pro system (Thermo Fisher Scientific) in a 386-well plate format. House-keeping gene ACTB served as a control for normalization. Relative abundance of the transcripts was examined using 2-ΔΔCT. Sequences of the primers used in this study are listed in Table 4.

TABLE 4

| Primer | Sequence | SEQ ID NO. |
|---|---|---|
| Actb_forward | GGCCAACCGTGAAAAGATGA | 9 |
| Actb_reverse | TACGACCAGAGGCATACAGG | 10 |
| H2-K1_forward | CCCTGTGAGCCTATGGACTC | 11 |
| H2-K1_reverse | TGTGGAAGGGAAGACAGAGC | 12 |
| H2-D1_forward | ACATCCAGAGCCCTCAGTTC | 13 |
| H2-D1_reverse | GGCTCCACAGTTCTTCACAC | 14 |

### Immunoblot

Cells were lysed in RIPA buffer (Thermo Fisher Scientific; catalog #: 89901) supplemented with a protease inhibitor cocktail (Cell Signaling Technology; catalog #: 5871). The lysates were then sonicated for a total of 30 seconds. Cell debris was then removed by centrifugation, and the protein concentration was measured with the Pierce™ BCA Protein Assay Kit (Thermo Fisher Scientific; catalog #: 23227) according to the manufacturer's instructions. The samples were next loaded and separated by SDS-PAGE and transferred to PVDF membrane (Merck; catalog #: IPVH00010). After blocking with 5% (w/v) BSA or milk, the membrane was incubated with primary antibody at 4° C. overnight and washed three times with TBST (0.1% Tween 20 in TBS). The membrane was then blotted with horseradish peroxidase (HRP)-linked secondary antibody and washed with TBST for another three times. Chemiluminescent substrate (Thermo Fisher Scientific; catalog #: 34096) was then applied to the membrane for visualization on the ChemiDoc™ XRS+Imaging System (Bio-Rad). Antibodies used in this study include anti-PIKfyve (R&D Systems; catalog #: AF7885), anti-LC3A/B (Cell Signaling Technology; catalog #: 12741S), anti-GAPDH (Cell Signaling Technology; catalog #: 3683S), anti-Vinculin (Cell Signaling Technology; catalog #: 4650S), anti-H3 (Cell Signaling Technology; catalog #: 3638S), anti-MHC-I (Abcam; catalog #: ab70328), anti-ATG5 (Cell Signaling Technology; catalog #: 12994S), anti-ATG7 (Cell Signaling Technology; catalog #: 8558S), anti-ovalbumin (Thermo Fisher Scientific; catalog #: MA515307), and anti-beta-2-microglobulin (Cell Signaling Technology; catalog #: 59035S).

### Immunohistochemistry

Following paraffin-embedding, xenograft tissues were sectioned and deparaffinized, followed by rehydration. Antigen retrieval was then performed in sodium citrate buffer (10 mM sodium citrate, 0.05% Tween 20, pH 6.0), followed by treatment of 3% H₂O₂ and blocking in PBS containing 5% goat serum. The sections were then incubated with the primary antibody, anti-HLA class 1 ABC (EMR8-5; Abcam; catalog #: ab70328) overnight at 4° C. The sections were then washed with PBST (0.1% Tween 20 in PBS), followed by incubation with the secondary antibody, Goat Anti-Rabbit IgG (H+L)-HRP Conjugate (Bio-Rad; #1706515). The sections were further washed with PBST, counterstained with hematoxylin, and imaged using a microscope. The quantification for MHC-I levels was performed after deconvoluting the brown layer from the image using the Fiji Is Just ImageJ downloadable online.

### RNA-Sequencing

The Eukaryote Total RNA Nano kit (Agilent Technologies; catalog #: 5067-1511) was used to check the quality of RNAs on an Agilent bioanalyzer. A total of 800 ng RNA was then used for library preparation with the KAPA RNA Hyper+RiboErase HMR kit (Roche Diagnostics; Catalog #: 08098140702), following the instructions from the user manual. Briefly, the ribosomal RNA was removed by enzymatic digestion, and then the RNA was fragmented to around 200-300 bp with heat in fragmentation buffer. Synthesis of cDNA was conducted with reverse transcriptase and random primer, and then the second strand was synthesized to generate double-stranded cDNA. Following the repair of the DNA ends, NEB adapter was ligated, and the DNA are amplified with the KAPA HiFi HotStart mix and NEB dual barcode. Quality of the library was checked using the bioanalyzer with the Agilent DNA 1000 Kit (Agilent Technologies; catalog #: 5067-1504), and the sequencing was performed using NovaSeq 6000. The RNA-sequencing data was analyzed with packages limma and edgeR See, e.g., Ritchie, M. E. et al., Nucleic Acids Res. 2015; 43(7):47; Phipson, B. et al., Ann. Appl. Stat. 2016; 10(2):946-963; Robinson, M. D. et al., Bioinformatics 2010; 26(1):139-40. Gene set enrichment analysis was conducted on the ranked gene set (log 2 fold-change*-log 10(p-value)) using the fgsea package. The analyzed hallmark pathways were collected from the Molecular Signatures Database See, e.g., Subramanian, A. et al., Proc. Natl. Acad. Sci. USA 2005; 102(43):15545-15550; Liberzon, A. et al., Cell Syst. 2015; 1(6):417-425. Mouse-specific bulk deconvolution tools, seq-ImmuCC and mMCP-counter, were used to estimate the differential relative abundance of CD8⁺ T cells See, e.g., Guo, J. Y. et al., Front. Immunol. 2018; 9:1286; Petitprez, F. et al., Genome. Med. 2020; 12(1):86.

### Human Studies

Acquisition and utilization of all clinical data in this study were approved by the U-M Institutional Review Board. Patients were recruited via the U-M Hospital, Ann Arbor, MI, USA, to receive ICB therapy. Patients enrolled in the MI-ONCOSEQ sequencing program at the Michigan Center for Translational Pathology (MCTP) and that had sequencing data from pre-treatment tumors were used for prediction of treatment response and survival See, e.g., Yu, J. et al., Nat. Med. 2021; 27(1):152-164; Robinson, D. R. et al., Nature 2017; 548(7667):297-303; Wu, Y. M. et al., Cell 2018; 173(7):1770-1782; Wang, W. et al., Nature 2019; 569(7755):270-274. Survival times were calculated from the beginning of therapy. RECIST1.140 criteria were used to establish treatment response. Patients with pseudo progression (imRECIST criteria) were excluded See, e.g., Hodi, F. S. et al., J. Clin. Oncol. 2018; 36(9):850-858. Sequencing was conducted with approved protocols in the MCTP Clinical Laboratory Improvement Amendments-compliant sequencing laboratory as described previously See, e.g., Robinson, D. R. et al., Nature 2017; 548(7667):297-303; Wu, Y. M. et al., Cell 2018; 173(7):1770-1782; Parolia, A. et al., Nature 2019; 571(7765):413-418. Briefly, total RNA was purified with the AllPrep DNA/RNA/miRNA kit (Qiagen) and sequenced with the exome-capture transcriptome system in paired-end method on a HiSeq 2000 or HiSeq 2500 (Illumina). Quality control, alignment, and expression quantification was achieved with CRISP, the standard clinical RNA-Seq pipeline See, e.g., Cieslik, M. et al., Genome Res. 2015; 25(9):1372-1381. Data were then analysed with the R package edgeR See, e.g., Robinson, M. D. et al., Bioinformatics 2010; 26(1):139-40.

### Analysis of Public Data

Public data from immunotherapy-treated cohorts with PIKfyve mRNA expression were downloaded from the Kaplan Meier plotter See, e.g., Lanczky, A. et al., J. Med. Internet Res. 2021; 23(7):27633. Data from pre-treatment samples were used for the prediction of survival. Best cutoff was selected in the dichotomized analysis.

### Statistical Analysis

All data points were acquired with distinct samples rather than acquiring with repeated assessments. Data were analyzed and plotted with Prism version 8 (GraphPad Software; San Diego, CA). Data were presented as means±s.d. or ±s.e.m., as stated in the FIG. legends. Statistical significance was determined with a p-value less than 0.05, unless stated otherwise.

### Example 1

### Genetic or Pharmacologic Inhibition of PIKfyve Induces MHC-I Surface Expression

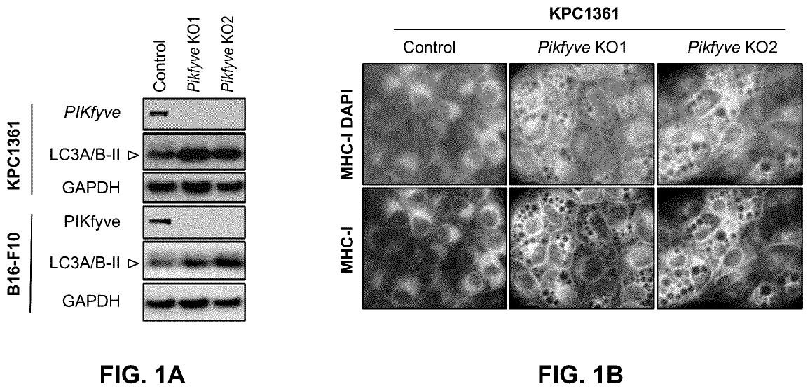

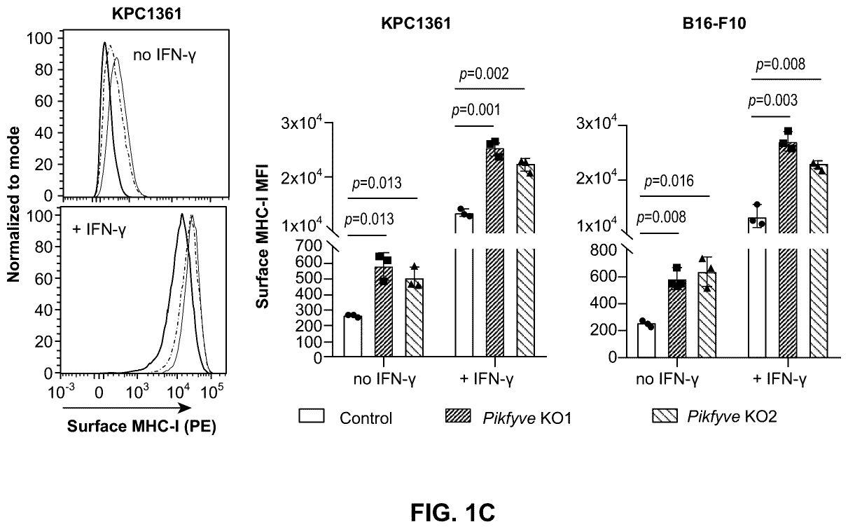

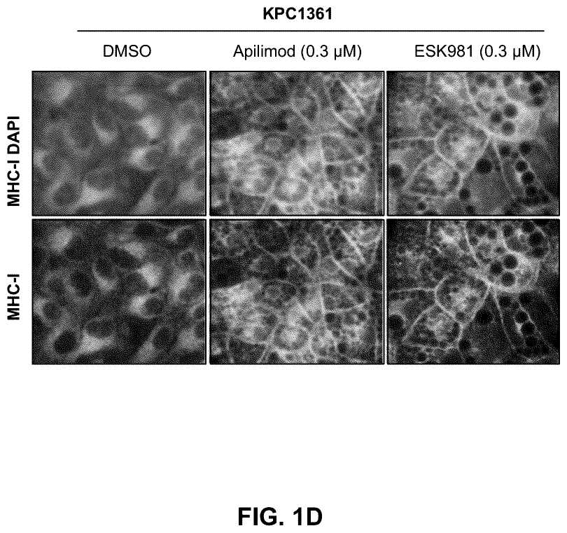

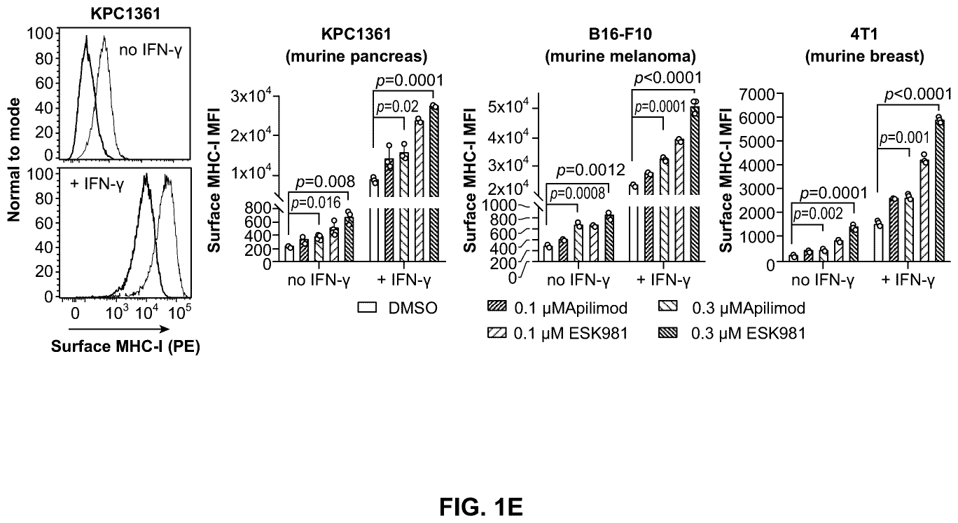

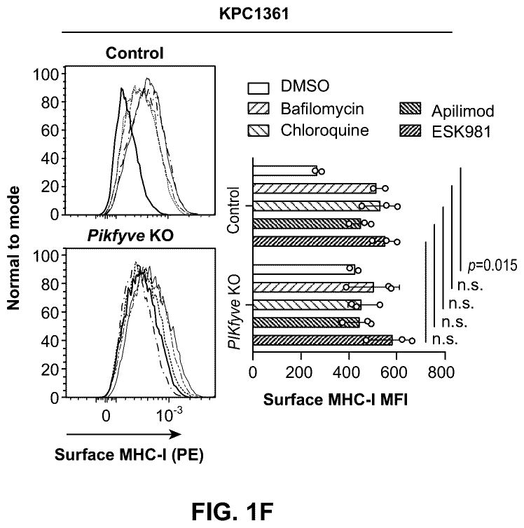

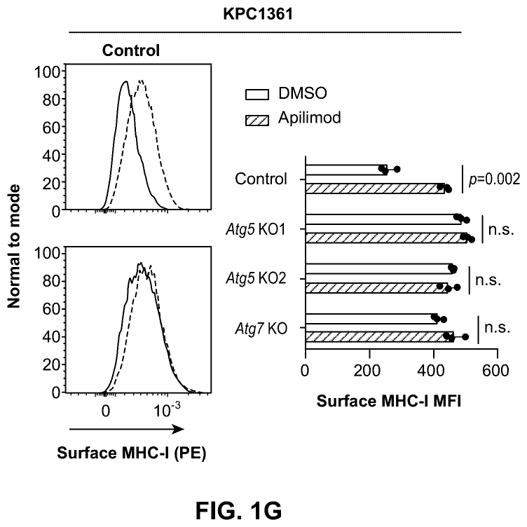

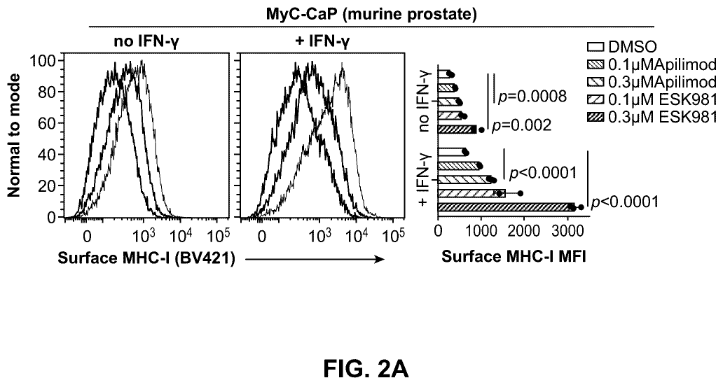

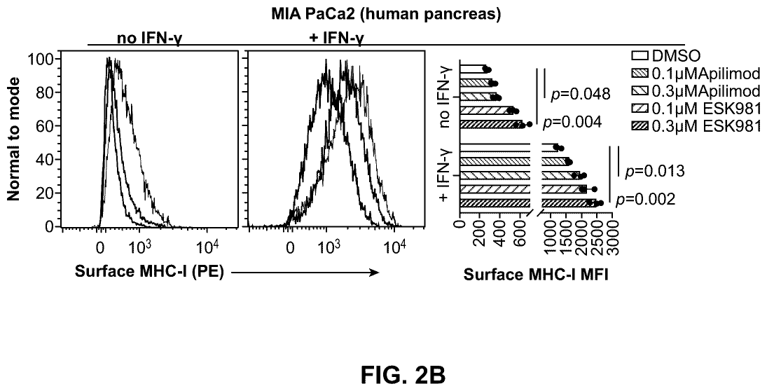

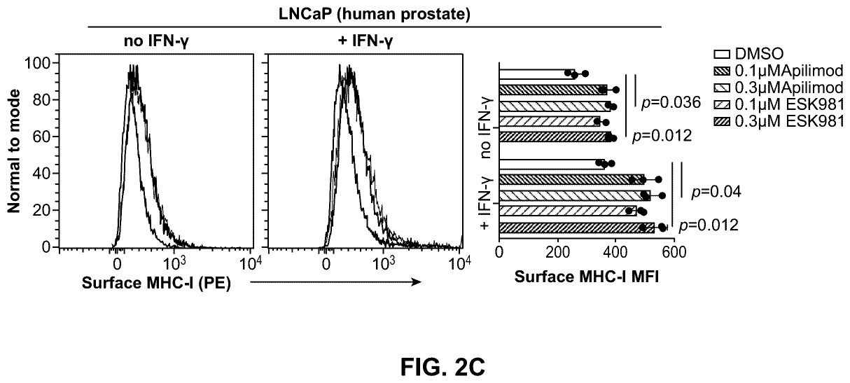

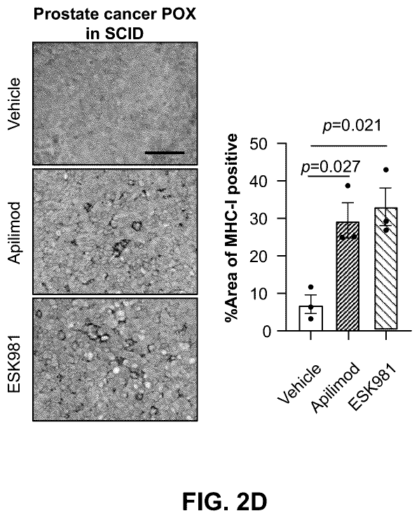

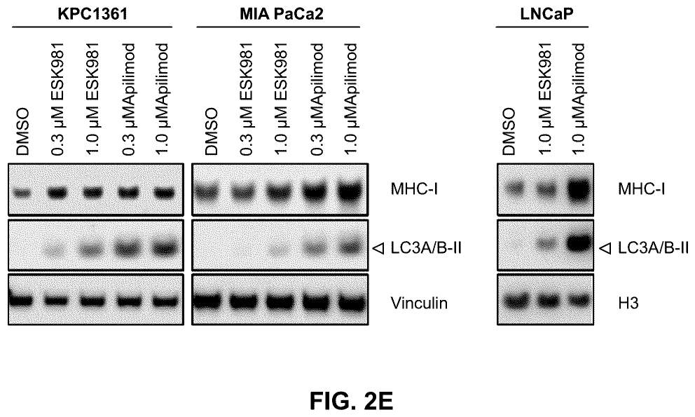

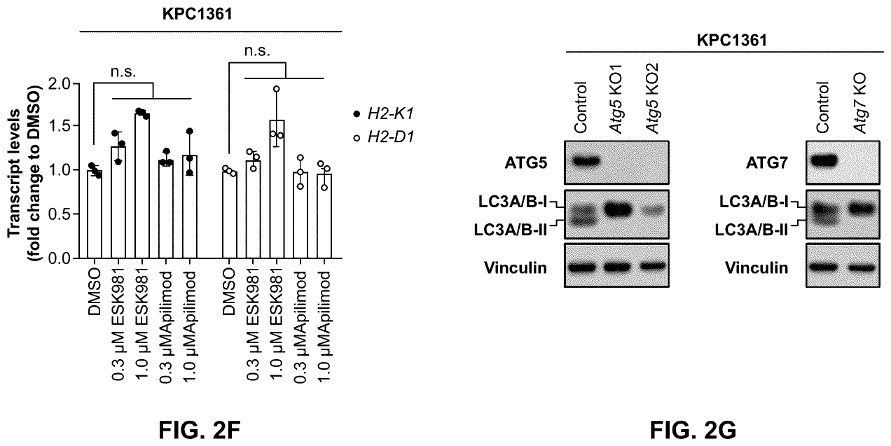

To examine whether targeting PIKfyve induces MHC-I surface expression, Pikfyve was first knocked out in different murine cancer cell models—KPC1361 derived from a pancreatic tumor of a genetically engineered mouse model (LSL-Kras^{G12D/+}; LSL-Trp53^{R172H/+}; Pdx1-Cre) and the melanoma model B16-F10 (FIG. 1A). Consistent with previous reports, See, e.g., Qiao, Y. et al., Nat. Cancer 2021; 2:978-993; Sharma, G. et al., Autophagy 2019; 15(10):1694-1718, Pikfyve depletion led to accumulation of the lipidated form (LC3A/B-II) of MAP1LC3A/B protein (LC3A/B) (FIG. 1A) and formation of vacuoles in vitro in the cancer cells (FIG. 1B). Employing immunofluorescence, it was found that knockout of Pikfyve resulted in increased cell surface expression of MHC-I (FIG. 1B). To quantify the surface expression of MHC-I, flow cytometry analysis was employed to confirm that Pikfyve depletion elevated both constitutive and IFN-γ-induced surface expression of MHC-I (FIG. 1C). Next, the effects of apilimod and ESK981 were examined. Apilimod or ESK981 treatment also increased cell surface expression of MHC-I (FIG. 1G), and upregulated both constitutive and induced surface expression of MHC-I in various murine cancer types, including KPC1361 (pancreas), B16-F10 (melanoma), 4T1 (breast), and MyC-CaP (prostate), in a dose-dependent manner (FIG. 1E and FIG. 2A). The increased MHC-I surface expression by PIKfyve inhibition was also observed in various human cancer models, including MIA PaCa2, LNCaP, and prostate cancer patient-derived xenografts (FIG. 2B-D). In line with previous reports, treatment with the PIKfyve inhibitors also led to LC3A/B-II accumulation (FIG. 2E) and vacuole formation (FIG. 1D) See, e.g., Qiao, Y. et al., Nat. Cancer 2021; 2:978-993; Sharma, G. et al., Autophagy 2019; 15(10):1694-1718. PIKfyve has been shown to regulate the formation of autolysosomes and, thus, protein degradation See, e.g., Gayle, S., et al., Blood 2017; 129(13):1768-1778; Sharma, G. et al., Autophagy 2019; 15(10):1694-1718. It was observed that total protein levels, but not mRNA levels, of MHC-I were upregulated by PIKfyve inhibition (FIG. 2E-F). To evaluate whether the action of PIKfyve inhibitors was on-target, Pikfyve-wildtype or Pikfyve-null cancer cells were treated with apilimod or ESK981. It was found that upregulation of MHC-I surface expression by PIKfyve inhibition was only observed in the wildtype cells but not Pikfyve-null cells (FIG. 1F). These data confirmed that the action of PIKfyve inhibitors was on-target.

PIKfyve inhibition has previously been shown to impair autophagy. See, e.g., Qiao, Y. et al., Nat. Cancer 2021; 2:978-993; Sharma, G. et al., Autophagy 2019; 15(10):1694-1718. Here, it was determined whether inhibition of PIKfyve elevated MHC-I surface expression by perturbing autophagic flux. Autophagy was disrupted with bafilomycin or chloroquine in Pikfyve-wildtype or Pikfyve-null cancer cells. In line with what has been reported, it was observed that inhibition of autophagy by bafilomycin or chloroquine induced MHC-I surface expression in the Pikfyve-wildtype cells (FIG. 1F) See, e.g., Yamamoto, K. et al., Nature 2020; 581(7806):100-105. While knockout of Pikfyve alone elevated MHC-I surface expression, it failed to further upregulate the levels of MHC-I surface expression upon autophagy inhibition (FIG. 1F). These data indicate that the elevated MHC-I surface expression by PIKfyve inhibition was achieved via autophagy impairment. Moreover, autophagy was disrupted with a genetic approach by depleting Atg5 or Atg7. Successful knockout of Atg5 or Atg7 was indicated by complete loss of ATG5 or ATG7 protein expression, respectively, and impaired LC3A/B-II formation (FIG. 2G) See, e.g., Yoshii, S. R. et al., Dev. Cell 2016; 39(1):116-130; Mizushima, N. et al., J. Cell Biol. 2001; 152(4):657-68; Guo, J. Y. et al., Genes Dev., 2013; 27(13):1447-1461. As expected, it was observed that depletion of Atg5 or Atg7 also resulted in increased MHC-I surface expression (FIG. 1G). While PIKfyve inhibition elevated MHC-I surface expression in wildtype cells, it did not further increase surface expression of MHC-I in the Atg5-null or Atg7-null cancer cells, confirming that PIKfyve inhibition upregulated MHC-I surface expression by impairing autophagy (FIG. 1G).

### Example 2

### Genetic or Pharmacologic Inhibition of PIKfyve Upregulates Antigen Presentation and Enhances Cancer Cell Killing by CD8⁺ T Cells

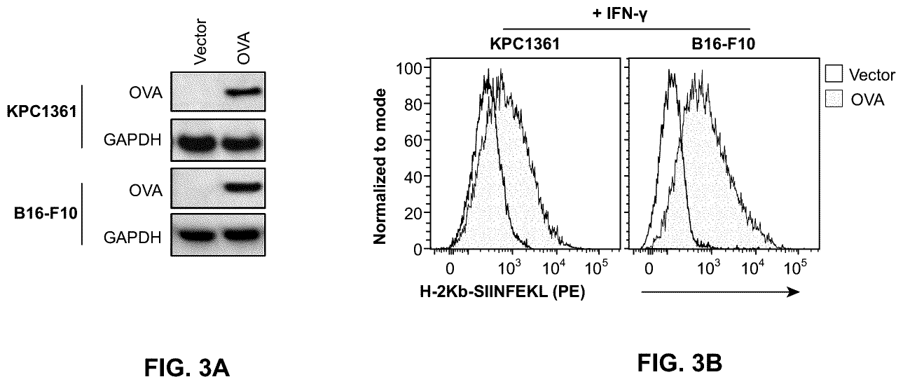

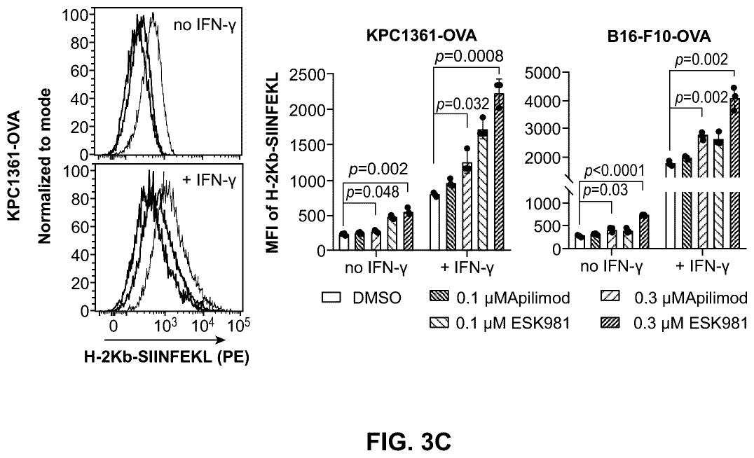

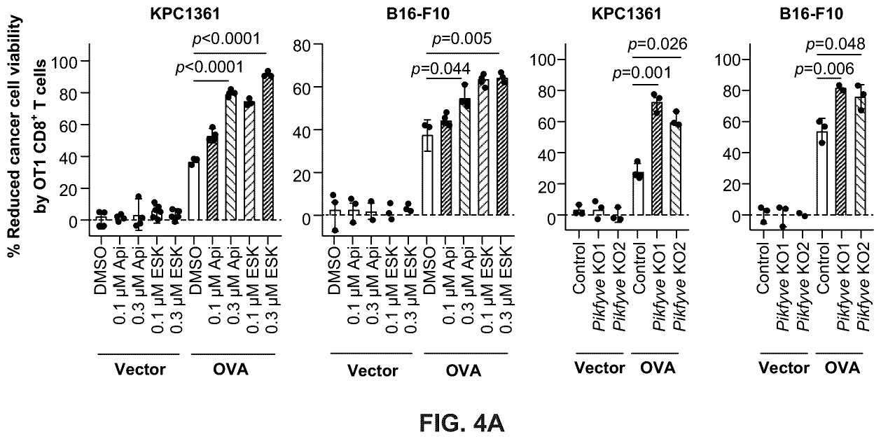

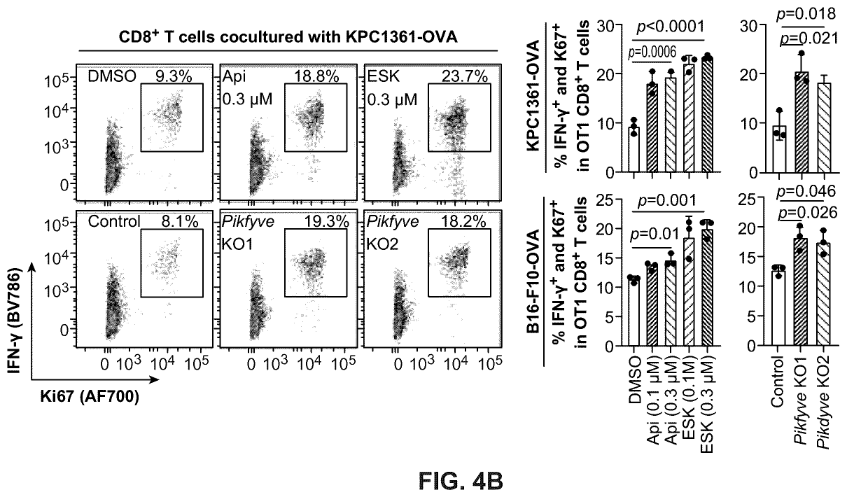

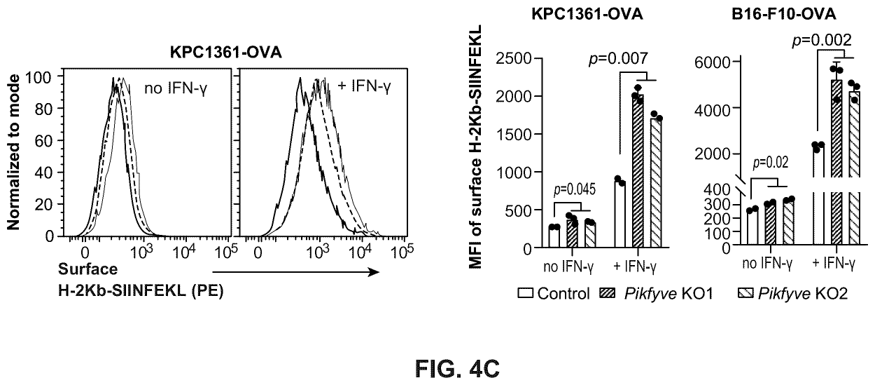

Next, it was examined whether pharmacologic inhibition or genetic depletion of Pikfyve led to enhanced CD8⁺ T cell-mediated cancer cell killing. Ovalbumin-expressing (OVA) KPC1361 and B16-F10 cells (FIG. 3A-B) were generated and co-cultured with activated CD8⁺ T cells isolated from OT1 mice. It was found that the OT1 CD8⁺ T cells had virtually no effect on the control cancer cells expressing the empty vector but killed the OVA-expressing cancer cells (FIG. 4A). Importantly, pharmacologic inhibition or genetic depletion of Pikfyve significantly enhanced the OVA cancer cell killing mediated by CD8⁺ T cells (FIG. 4A). Concordantly, an increase in IFN-γ expression and Ki-67 proliferating CD8⁺ T cells in co-culture following pharmacologic inhibition or genetic depletion of Pikfyve was observed (FIG. 4B). The antigen presentation in the OVA cancer cells was further evaluated with flow cytometry, using an antibody detecting an OVA-peptide (SIINFEKL)-bound MHC-I (H-2Kb). It was found that genetic or pharmacologic inhibition of PIKfyve resulted in upregulation of antigen presentation in the cancer cells (FIG. 4C and FIG. 3C). Collectively, genetic or pharmacologic inhibition of PIKfyve upregulates antigen presentation and enhances cancer cell killing mediated by CD8⁺ T cells.

### Example 3

### Loss of Pikfyve Retards Tumor Progression in a CD8⁺ T Cell- and MHC Class I-Dependent Manner

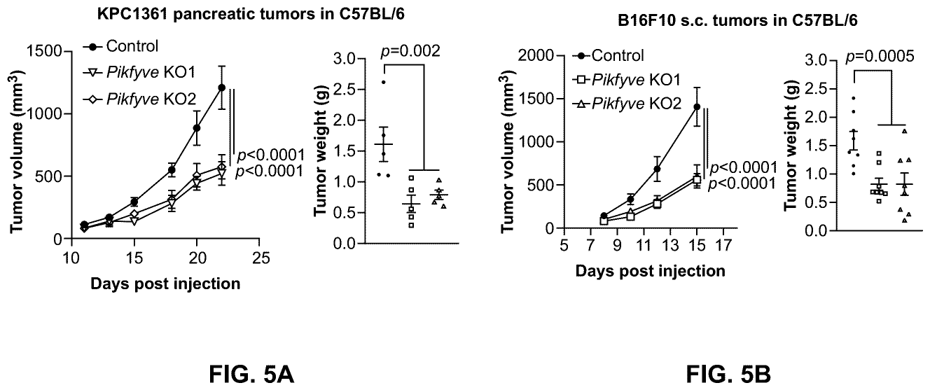

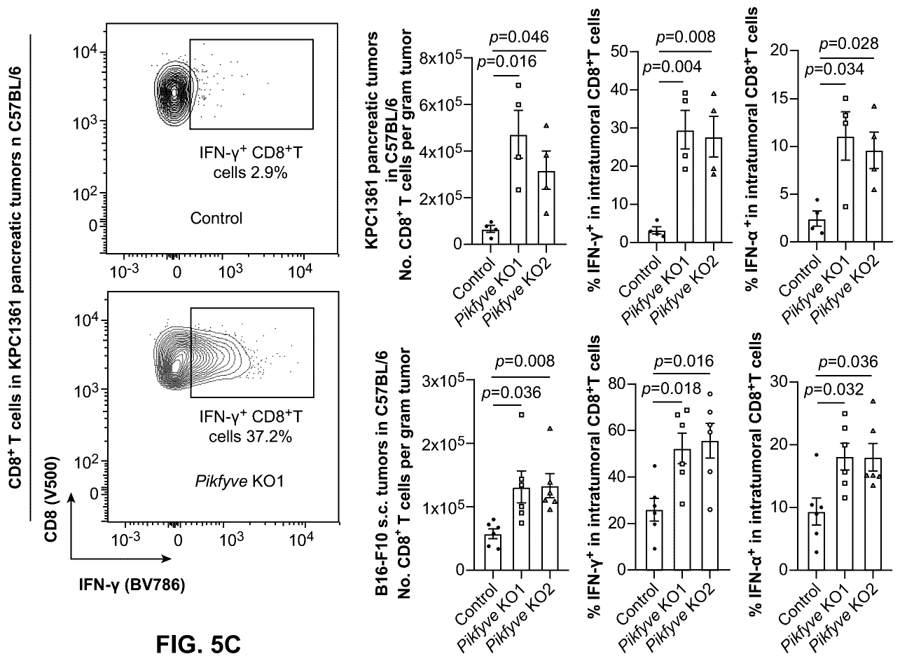

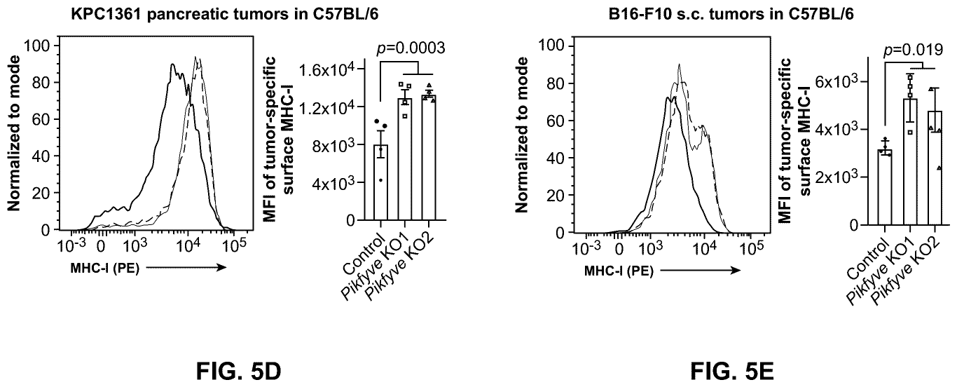

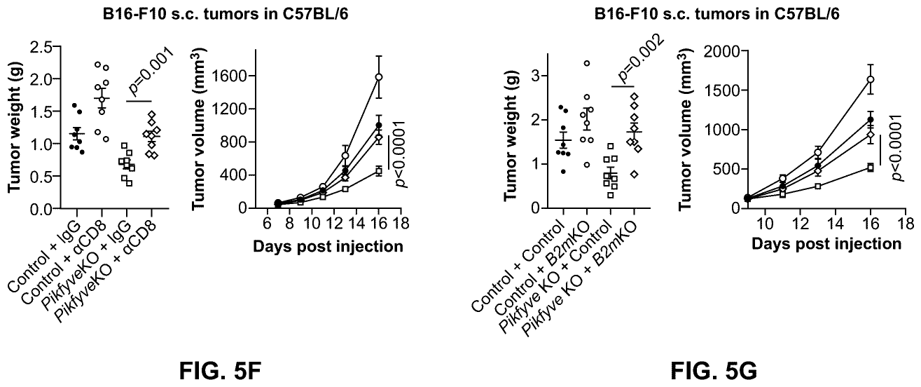

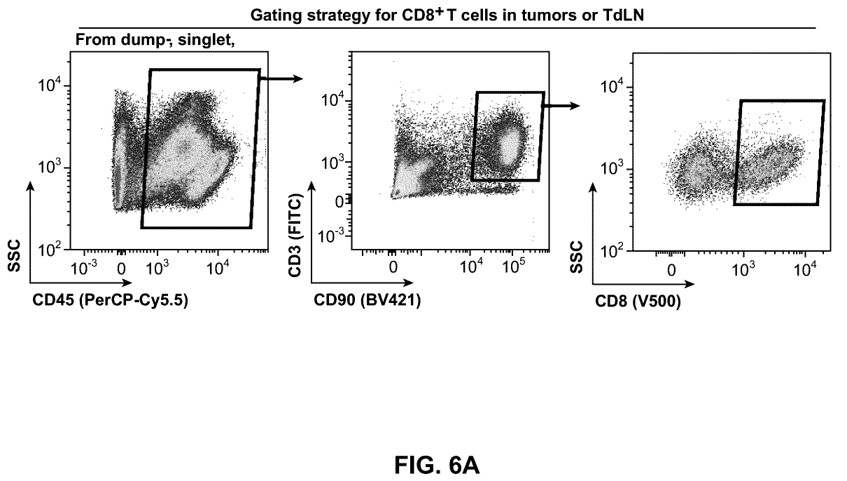

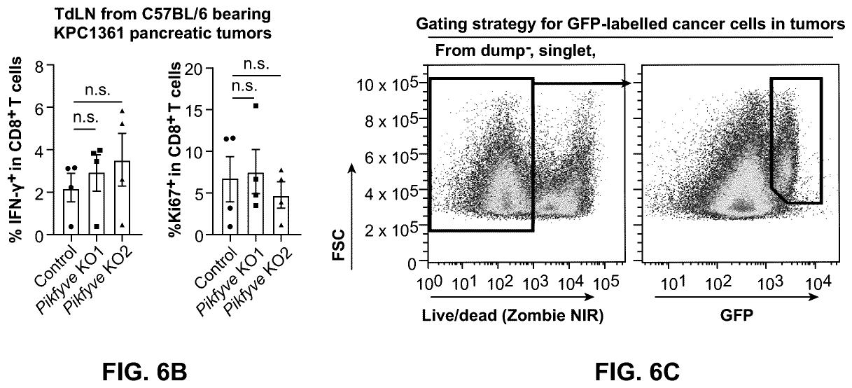

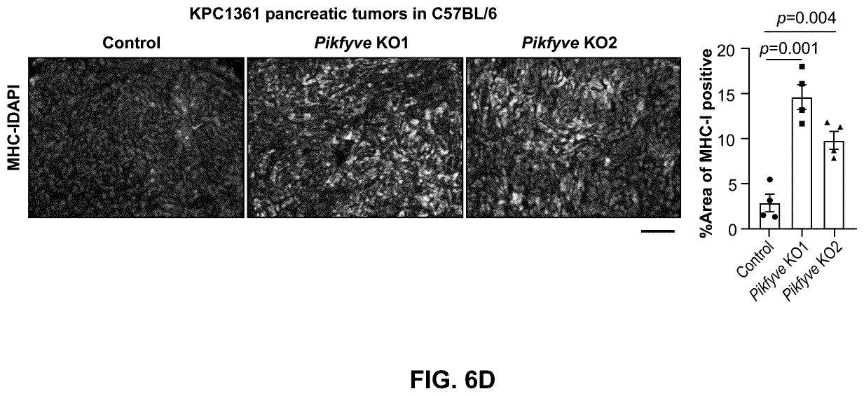

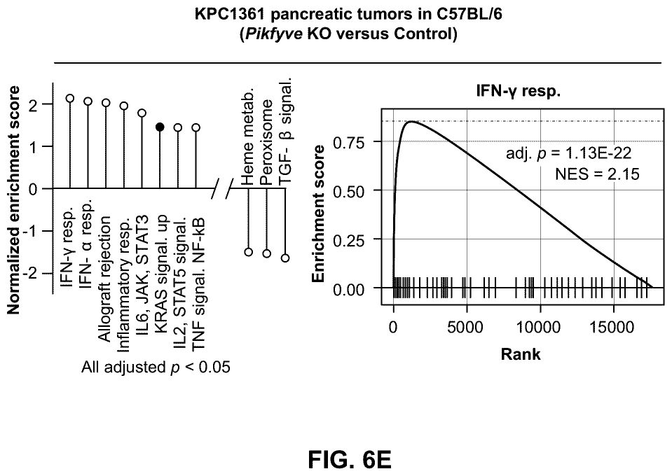

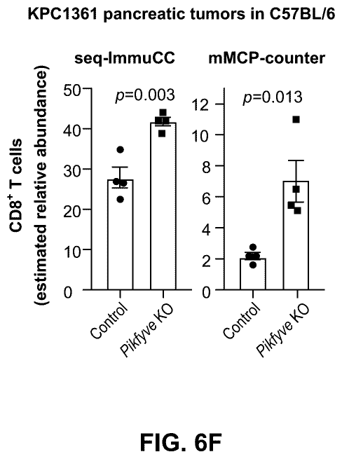

The effect of Pikfyve loss in vivo by injecting the Pikfyve knockout cancer cells into their immunocompetent syngeneic hosts was evaluated (FIG. 5A-B). Depletion of Pikfyve was found to strongly retarded tumor progression in both the pancreatic (KPC1361) and melanoma (B16F10) models, compared to the Pikfyve-wildtype control (FIG. 5A-B). The amount of total and activated CD8⁺ T cells in the tumors was measured and it was observed that knockout of Pikfyve increased the amount of intratumoral CD8⁺ T cells and the proportion of activated CD8⁺ T cells (FIG. 5C and FIG. 6A). No significant change of CD8⁺ T cell activation in the tumor-draining lymph nodes was observed (TdLN; FIG. 6B), suggesting that Pikfyve-loss in malignant cells affects quantity and functionality of CD8⁺ T cells in the tumor microenvironment, rather than CD8⁺ T cell priming in TdfLN. Flow cytometry was further performed to measure the surface expression of MHC-I in cancer cells in vivo using fluorescent-labelled cancer cells (FIG. 6C). Consistent with what was observed in vitro, Pikfyve depletion led to upregulation of MHC-I surface expression in tumor cells (FIG. 5D-E). Importantly, upregulation of tumor-specific MHC-I expression by Pikfyve-loss was also validated using immunofluorescence (FIG. 6D). Finally, bulk RNA-sequencing was performed and identified multiple immune-related pathways enriched in the Pikfyve-loss tumors when compared to the Pikfyve-wildtype, with IFN-γ response being the top pathway (FIG. 6E). Estimation of CD8⁺ T cell abundance with different models, seq-InmmuCC or MMCP-counter, on the bulk RNA-sequencing data also showed an increase of intratumoral CD8⁺ T cells (FIG. 6F), consistent with the flow cytometry data See, e.g., Guo, J. Y. et al., Front. Imunuol. 2018; 9:1286; Petitprez, F, et al., Genome. Med. 2020; 12(1):86. Collectively, these data suggest that Pikfyve-depletion controlled tumor growth in an immune-dependent manner.

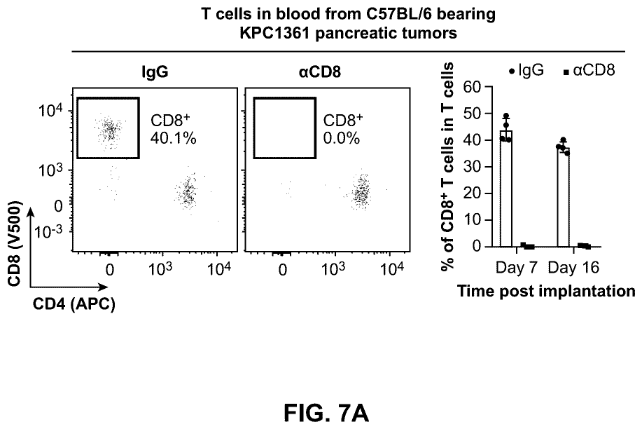

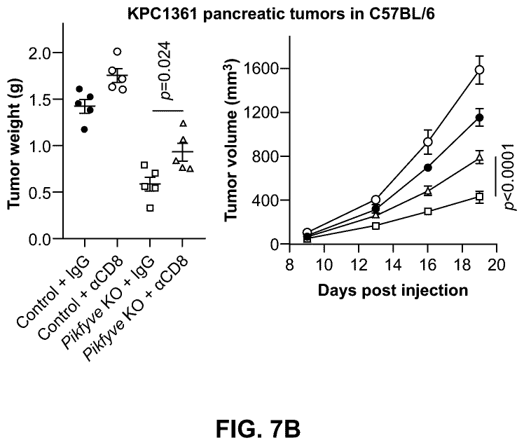

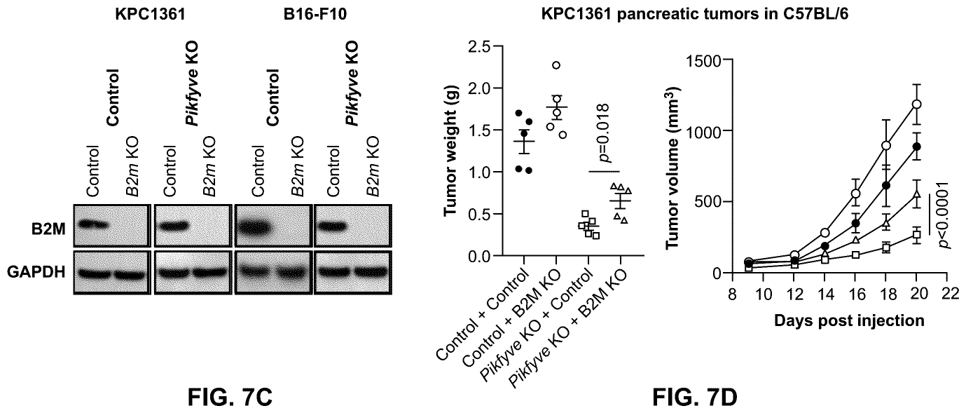

The increase of activated CD8⁺ T cells in Pikfyve-knockout tumors prompted the evaluation or whether CD8⁺ T cells were essential for the reduction of tumor growth by Pikfyve-knockout. CD8⁺ T cells were depleted in the syngeneic hosts and evaluated the growth of tumors established with the Pikfyve-knockout cancer cells. Successful depletion of CD8⁺ T cells was determined by flow cytometry (FIG. 7A). It was observed that CD8⁺ T cell depletion significantly rescued the progression of tumors derived from Pikfyve-knockout cancer cells (FIG. 5F and FIG. 7B). These data highlight that the tumor control mediated by Pikfyve-loss was CD8⁺ T cell-dependent. Next, it was sought to determine if the upregulation of MHC-I surface expression by Pikfyve-depletion was functional in tumor control, and B2m was knocked out in the Pikfyve-wildtype or Pikfyve-loss cancer cells (FIG. 7C). It was observed that loss of B2m significantly rescued the progression of tumors derived from Pikfyve-null cancer cells (FIG. 5G and FIG. 7D), supporting that the tumor control mediated by Pikfyve-loss was also mediated by tumor-specific MHC-I expression. Collectively, these data underscored that tumor control by Pikfyve-knockout was mediated by CD8⁺ T cells and tumor-specific MHC-I expression.

### Example 4

### PIKfyve Inhibition Enhances Efficacy of ICB, and PIKfyve Expression Predicts Response to ICB and Survival in ICB-Treated Cohorts

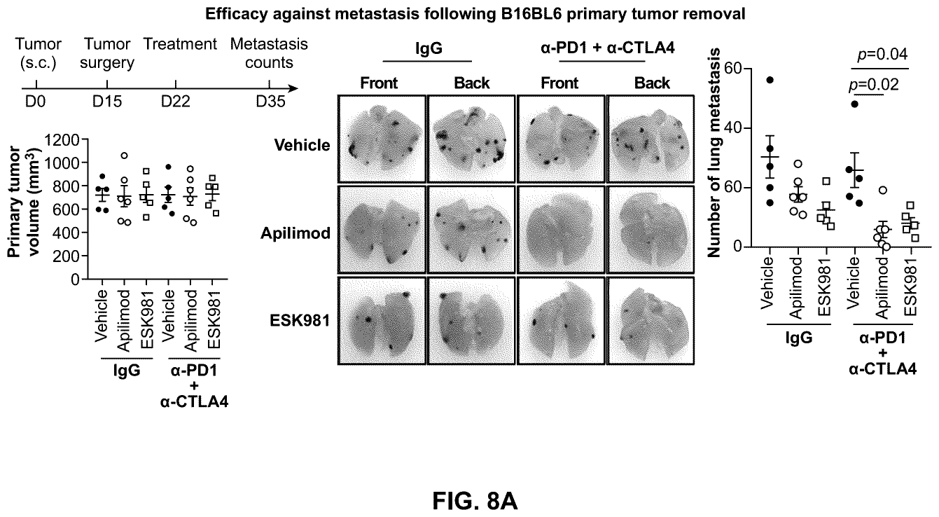

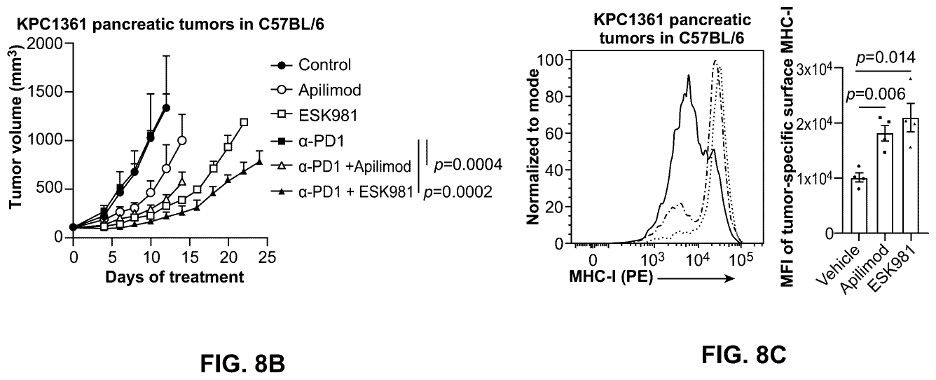

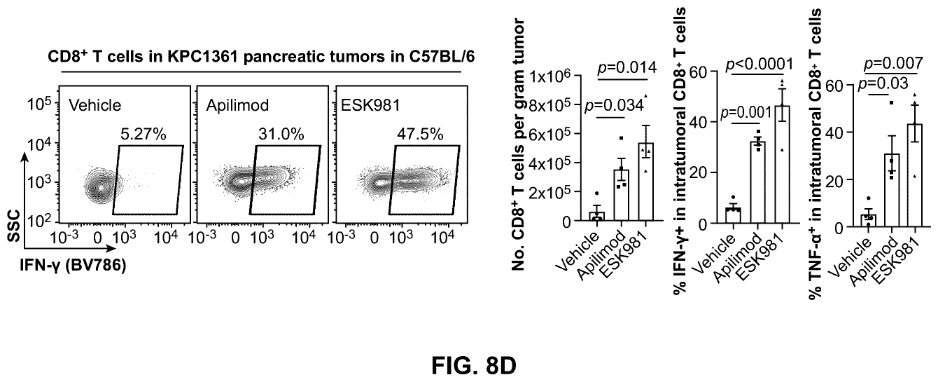

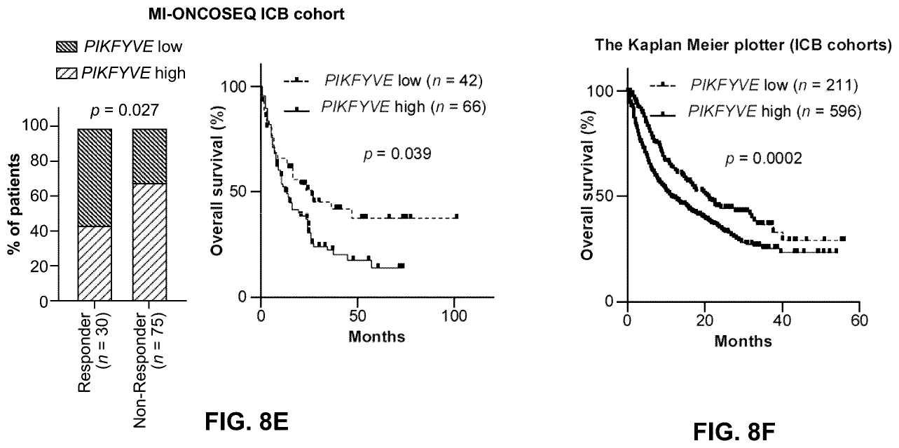

It was determined whether inhibition of PIKfyve improved efficacy of ICB. As metastasis is the major cause of cancer deaths, efficacy of combining PIKfyve inhibition and ICB against metastasis was first evaluated, using the B16-BL6 melanoma model that has a high inclination to form early lung metastatic dissemination after surgical removal of primary tumors See, e.g., Badrinath, S. et al., Nature 2022; 606(7916):992-998. Primary tumors derived from subcutaneous injection of B16-BL6 cells were surgically removed and randomized the mice into different treatment groups based on volumes of the primary tumors (FIG. 8A). The mice were treated with ICB (anti-PD-1 plus anti-CTLA-4) or PIKfyve inhibitor alone, or in combination, and sacrificed at endpoint for quantifying lung metastasis counts (FIG. 8A). As expected, in the control group, significant amounts of lung metastasis were observed at sacrifice (FIG. 8A). The B16-BL6 model was previously reported to be relatively resistant to ICB See, e.g., van Elsas A. et al., J. Exp. Med. 1999; 190(3):355-66; Curran, M. A. et al., Proc. Natl. Acad. Sci. USA 2010; 107(9):4275-80. In line with this, no significant reduction of metastasis in the ICB-treated group was observed (FIG. 8A). While the single agent PIKfyve inhibitors (apilimod or ESK981) exhibited reduction of lung metastasis, they also strongly enhanced the efficacy of ICB to inhibit metastasis (FIG. 8A). These data highlight the efficacy of combining PIKfyve inhibition and ICB against metastasis.

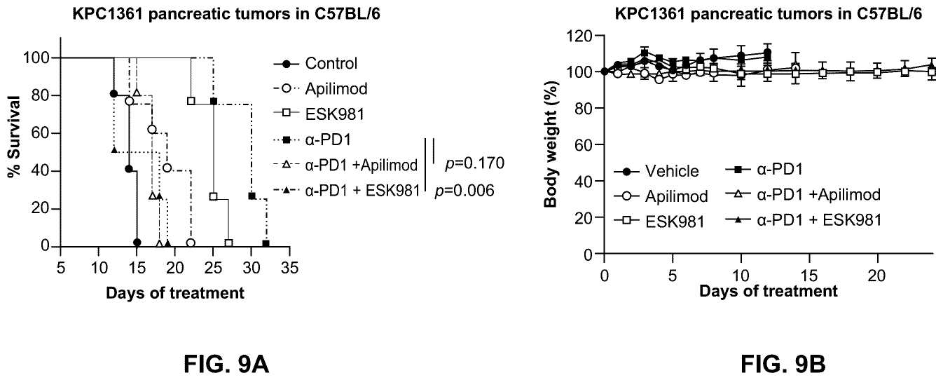

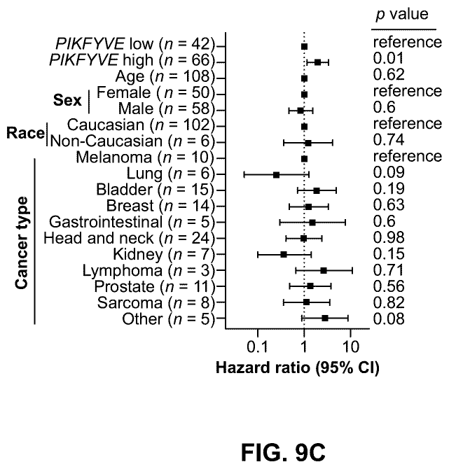

PIKfyve inhibition was observed to improve anti-PD-1 efficacy against syngeneic pancreatic tumors established with orthotopic injection of KPC1361 cells (FIG. 8B), which translated into improved overall survival (FIG. 9A). Importantly, PIKfyve inhibition by apilimod or ESK981 also led to upregulation of tumor-specific MHC-I surface expression (FIG. 8C) and an increase in intratumoral activated CD8⁺ T cells (FIG. 8D). PIKfyve inhibitor treatment showed a favorable safety profile as a single agent or in combination with anti-PD-1, as the treated mice showed no perceptible weight loss (FIG. 9B).

These data prompted the determination whether PIKfyve expression in human tumors predicts response to ICB and survival in ICB-treated cohorts. Using pre-treatment RNA-sequencing data from tumors obtained from a stage IV ICB-treated pan-cancer cohort (n=108; data not shown) at the University of Michigan (U-M), it was found that high pre-treatment PIKfyve expression was significantly associated with poorer response to ICB (FIG. 8E). Importantly, high pre-treatment PIKfyveexpression also significantly predicted poorer overall survival in this cohort (FIG. 8E). Cox regression analysis further revealed that high pre-treatment PIKfyve expression was associated with poorer clinical outcome (p=0.01) independently of age, sex, race, and cancer type (FIG. 9C). To validate this observation with public data, pre-treatment ICB-treated cohorts (n=807) comprised of various cancer types, including bladder (n=73), esophageal adenocarcinoma (n=73), melanoma (n=317), urothelial (n=348), and glioblastoma (n=14) was downloaded. This showed that high pre-treatment PIKfyve expression was prognostic of poor overall survival (FIG. 8F).

In summary, high pre-treatment PIKfyve expression is predictive of poorer response to ICB treatment, and prognostic of poorer overall survival in ICB-treated cohorts. Thus, in some embodiments of the present disclosure, PIKfyve expression serves as a biomarker for immune responses triggered by PIKfyve inhibition.

### Example 5

### PIKfyve Inhibition Enhances Efficacy of Adoptive Cell Therapy and Therapeutic Vaccine Therapy

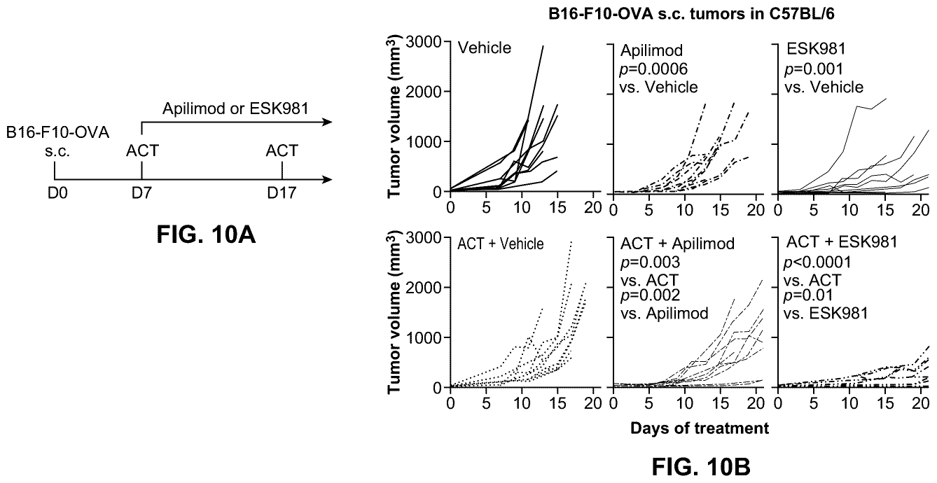

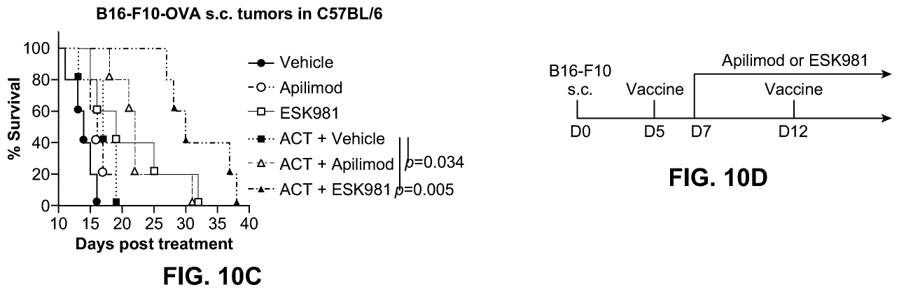

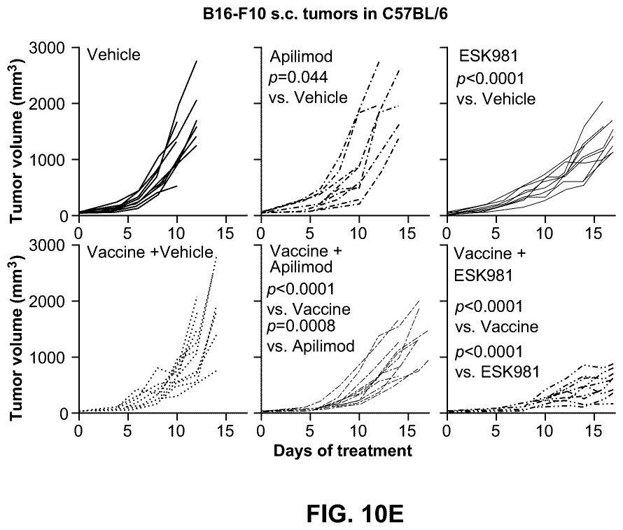

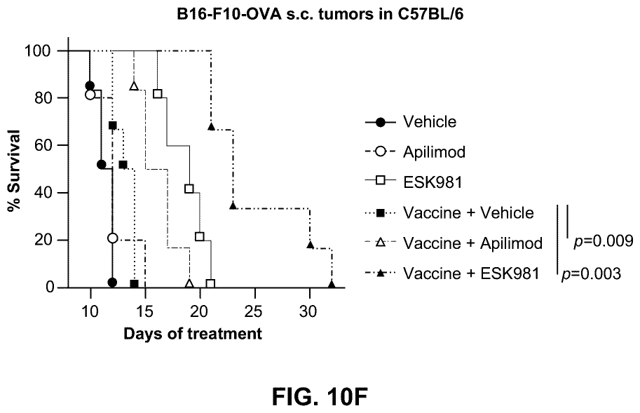

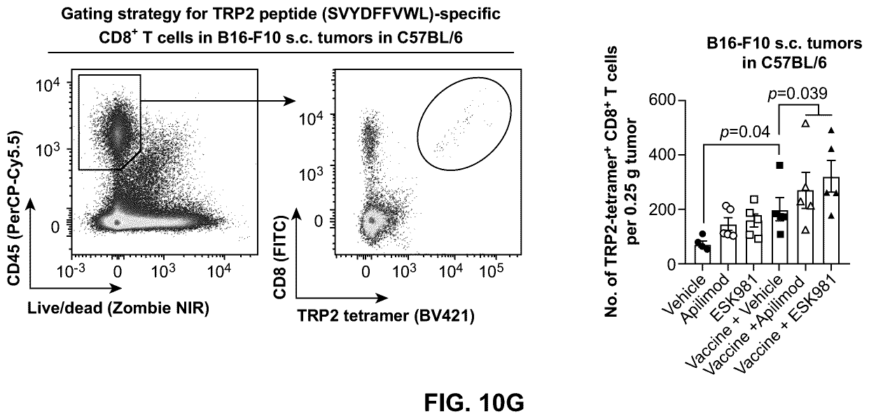

It was determined whether PIKfyve inhibition could enhance efficacy of other types of immunotherapies by first employing the OVA-expressing cancer cells (FIG. 3A-B). To assess if PIKfyve inhibition enhances efficacy of adoptive cell therapy (ACT), subcutaneous tumors derived from OVA-expressing B16-F10 cells were established and then CD8⁺ T cells derived from OT1 mice were infused into the tumor-bearing syngeneic hosts (FIG. 10A). Consistent with previous reports, the OT1 T cells alone exhibited marginal efficacy against tumors in this model (FIG. 10B), but inhibition of PIKfyve with apilimod or ESK981 strikingly enhanced efficacy of the ACT (FIG. 10B), significantly extending survival of the animals (FIG. 10C) See, e.g., Zhou, H. et al., J. Clin. Invest. 2022; 132 (16).

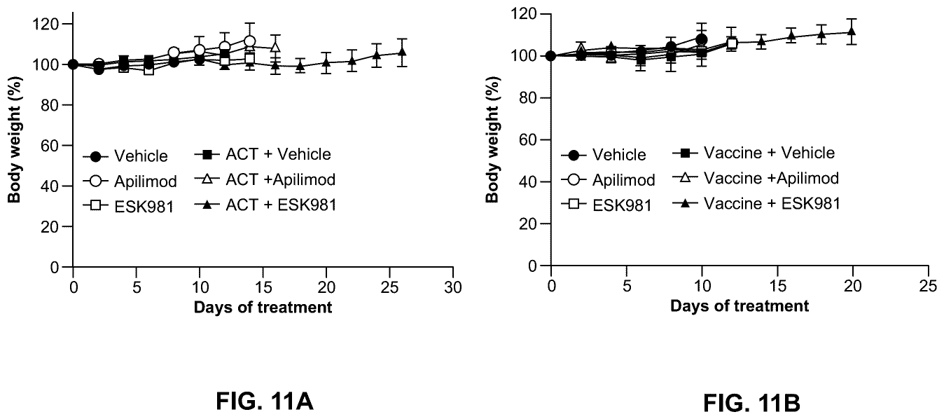

It was next interrogated whether PIKfyve inhibition could enhance efficacy of therapeutic vaccines. See, e.g., Sahin, U., et al., Nature 2020; 585(7823):107-112; Sahin, U. et al., Nature 2017; 547(7662):222-226; Frank, M. J. et al., J. Exp. Med. 2020; 217(9). Subcutaneous tumors with B16-F10 cells were established and treated with lethally irradiated B16-F10 cells plus poly(I:C) as an adjuvant into the tumor-bearing mice, five days post tumor inoculation (FIG. 10D). Although the therapeutic vaccine alone showed only a marginal effect on tumor control (FIG. 10E), inhibition of PIKfyve strongly improved efficacy of the vaccine (FIG. 10E), resulting in significant extension of survival (FIG. 10F). To examine whether the vaccine induced antigen-specific CD8⁺ T cells, the amount of intratumoral CD8⁺ T cells bearing a T cell receptor specific for an epitope (SVYDFFVWL) of tyrosinase-related protein 2 (TRP2), an enzyme expressed in B16-F10 cells was measured See, e.g., Bloom, M. B. et al., J. Exp. Med. 1997; 185(3):453-9; McWilliams, J. A. et al., J. Immunol. 2006; 177(1):155-61. It was observed that the vaccine treatment alone increased the amount of intratumoral antigen-specific CD8⁺ T cells, while treatment with PIKfyve inhibitors further increased the number of CD8⁺ T cells (FIG. 10G). Collectively, these data demonstrate that PIKfyve inhibition improves efficacy of ACT and vaccine therapy. Combining ACT or vaccine therapy with PIKfyve inhibitor treatment did not cause weight loss in the animals, indicating no observable toxicity (FIG. 11A-B).

These data suggest that treatment with apilimod or ESK981 enhances efficacy of immunotherapies that are CD8⁺ T cell- and MHC class I-dependent. Apilimod and ESK981 exhibit favorable safety profiles in preclinical models, both as single agents and in combination with the immunotherapies tested (FIG. 9B and FIG. 11A-B). It was observed that the efficacy of apilimod was weaker than ESK981 in many of models, even though the dose of apilimod was higher than ESK981. This could be, at least partially, explained by the reported instability of apilimod See, e.g., Ikonomov, O. C. et al., Toxicol. Appl. Pharmacol. 2019; 383:114771.

### Example 6

### DC PIKfyve Expression is Associated with ICB Efficacy

Protein kinases are important targets for cancer therapy. To explore the relevance of their gene targets in ICB-induced tumor immunity, the 25 most common and unique gene targets of Phase I/Phase II/FDA-approved kinase inhibitors, offered in a commercial screening library (ALK, AURKA, BTK, CSF1R, EGFR, FGFR1, FLT1, FLT3, IGF1, IGFR1, IKBKB, JAK1, JAK2, KDR, KIT, MET, MTOR, NTRK1, PDGFRA, PIKfyve, PTK2, RAF1, RET, SRC, and SYK), was first identified. See, e.g., Corsello, S. M. et al., Nat. Med. 2017:23: 405-408; Martin, J. K., 2nd et al., Cell 2020:181: 1518-1532; Tanaka, K. et al., Cancer Cell 2021:39: 1245-1261. The potential involvement of these 25 genes in treatment response in a clinical cohort of patients who received ICB and clinical bulk RNA-sequencing (RNA-seq) of their tumors at the University of Michigan as a part of their cancer treatment, was then explored (Table 5). See, e.g., Robinson, D. R. et al., Nature 2017:548: 297-303.

TABLE 5
Demographics of a clinical cohort of patients treated with ICB

| | | |
|---|---|---|
| Percent (n) | Hazard Ratio [95% CI] | P Value |

Age (time of sequenceing)

| Below Median | 50.0% | (n = 46) | 1 (reference level) | | — |
|---|---|---|---|---|---|
| Above Median | 50.0% | (n = 46) | 0.55 | [0.29, 1.03] | 0.06 |

Sex

| Female | 44.5% | (n = 41) | 1 (reference level) | | — |
|---|---|---|---|---|---|
| Male | 55.5% | (n = 51) | 0.92 | [0.46, 1.86] | 0.82 |

Immune checkpoint blockade Agent

| Atezolizumab | 9.8% | (n = 9) | 1 (reference level) | | — |
|---|---|---|---|---|---|
| Nivolumab | 27.2% | (n = 25) | 0.43 | [0.13, 1.40] | 0.16 |
| Pembrolizumab | 59.7% | (n = 55) | 0.58 | [0.23, 1.44] | 0.24 |
| Combination | 3.3% | (n = 3) | 0.43 | [0.065, 2.92] | 0.39 |

Cancer Type

| Melanoma | 9.8% | (n = 9) | 1 (reference level) | | — |
|---|---|---|---|---|---|
| Bladder | 16.3% | (n = 15) | 1.24 | [0.39, 3.86] | 0.70 |
| Breast | 14.1% | (n = 13) | 0.73 | [0.22, 2.34] | 0.60 |
| Gastrointestinal | 3.3% | (n = 3) | 1.20 | [0.20, 7.02] | 0.83 |
| Head and Neck | 20.7% | (n = 19) | 0.76 | [0.28, 2.05] | 0.60 |
| Kidney | 6.5% | (n = 6) | 0.27 | [0.053, 1.38] | 0.11 |
| Lung | 5.4% | (n = 5) | 0.10 | [0.011, 0.92] | 0.042 |
| Lymphoma | 3.3% | (n = 3) | 2.41 | [0.53, 10.99] | 0.25 |
| Prostate | 12.0% | (n = 11) | 1.16 | [0.39, 3.49] | 0.77 |
| Sarcoma | 4.3% | (n = 4) | 0.57 | [0.13, 2.40] | 0.44 |
| Other | 4.3% | (n = 4) | 1.97 | [0.53, 7.30] | 0.30 |

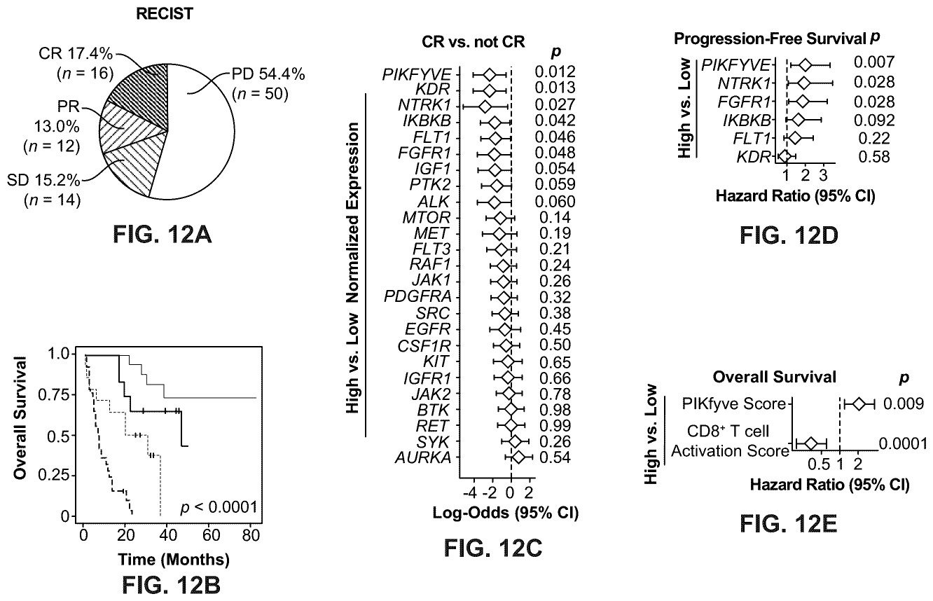

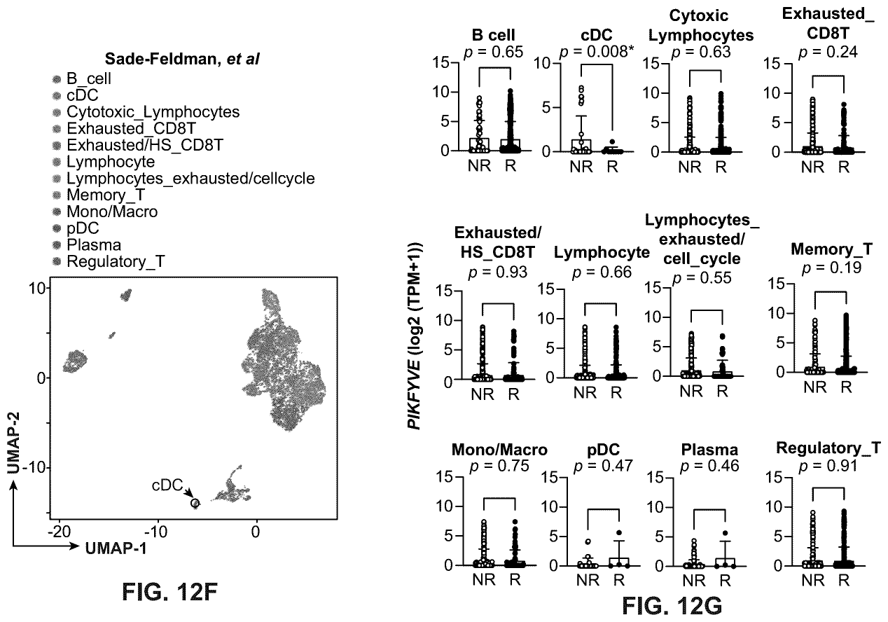

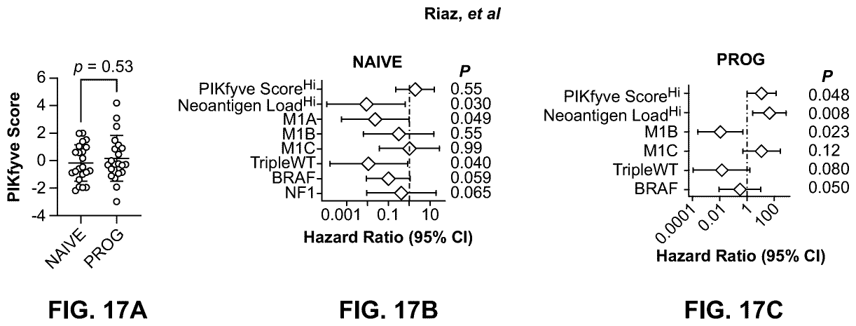

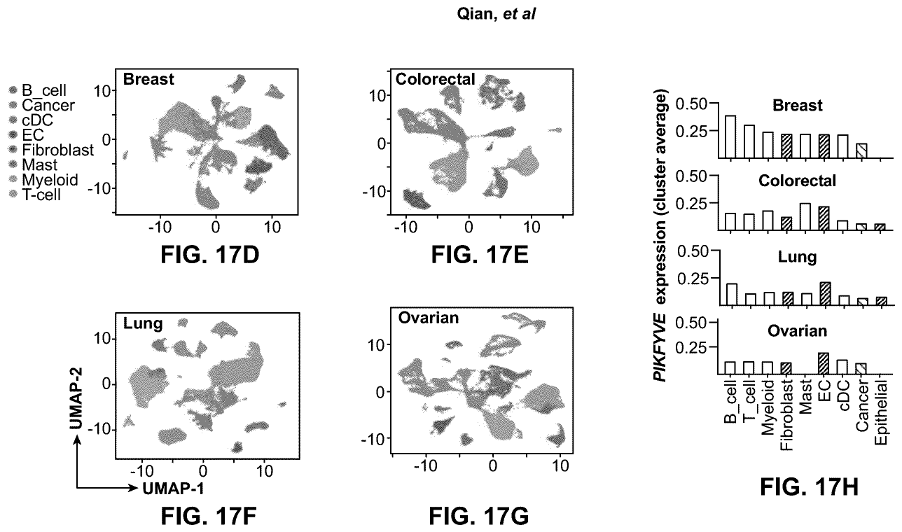

Seventeen percent of this cohort exhibited RECIST-defined complete response (CR) to ICB (FIG. 12A). See, e.g., Eisenhauer, E. A. et al., Eur. J. Cancer 2009:45: 228-247. As expected, the CR group had the best overall survival in the cohort (FIG. 12B). Within the group of kinase inhibitor gene targets evaluated, high pre-treatment PIKfyve, KDR, NTRK1, IKBKB, FLT1, or FGFR1 expression was each associated with lower odds of achieving CR when controlling for cancer type, biopsy site, age at the time of treatment initiation, and ICB agent (FIG. 12C). Amongst these targets, it was found that only high PIKfyve expression was independently associated with worse progression-free survival (PFS) upon treatment (FIG. 12D). Furthermore, a high pre-treatment CD8⁺ T cell activation score was associated with a better overall survival, whereas a high PIKfyve score had the opposite effect (FIG. 12E). See, e.g., Wang, W. et al., Nature 2019:569: 270-274; Yu, J. et al., Nat. Med. 2021:27: 152-164. Additionally, it was observed that pre-treatment PIKfyve scores were not different between patients with melanoma who were ICB-treatment-naïve (“NAÏVE”) or had previously progressed on treatment (“PROG”) (FIG. 17A). See, e.g., Riaz, N. et al., Cell 2017:171: 934-949. Interestingly, a high PIKfyve score was associated with worse overall survival in the PROG cohort when controlling for mutational subtype, stage of disease, and neoantigen load (FIG. 17B,C). Therefore, PIKfyve, encoding a cytosolic lipid kinase, may be a therapeutically targetable gene with relevance in patients receiving ICB. See, e.g., Krishna, S. et al., Dev. Cell 2016:38: 536-547; Vicinanza, M. et al., Mol. Cell 2015:57: 219-234; Giridharan, S. S. P. et al., Elife 2022:11.

It was then postulated that the correlation between PIKfyve and ICB-associated outcomes in cancer patients may depend on cell type. Utilizing a published single cell RNA-seq (scRNA-seq) datasets to assess this hypothesis, it was found that PIKfyve was expressed in immune cells across multiple cancer types (FIG. 17D-H). See, e.g., Qian, J. et al., Cell Res. 2020:30: 745-762. Importantly, in a study of patients with melanoma treated with ICB, pre-treatment expression of PIKfyve in conventional DCs (cDCs) was lower in responders (R) when compared to non-responders (NR) (FIG. 12F,G) See, e.g., Sade-Feldman, M. et al., Cell 2018:175: 998-1013. There were no differences in PIKfyve expression in any other immune cell type. In addition, a patient with endometrial cancer with CR had a lower expression of PIKfyve in their cDCs compared to nonresponders (FIG. 17I,J) See, e.g., Chow, R. D. et al., Cancer Discov. 2023:13: 312-331. Together, these data suggest that DCs act as an immune cell in which PIKfyve may play a significant role in cancer immunity and ICB-associated outcomes.

### Example 7

### PIKfyve Suppresses DC Function

Given these unexpected findings, it was sought to understand why patients with lower DC PIKfyve expression in tumors were inclined towards better clinical outcomes following ICB therapy. It was hypothesized that PIKfyve may have a direct and functional role in modulating DCs and DC-mediated immune response against tumors. To this end, genetic and pharmacologic manipulation of PIKfyve in DCs was performed to gain a comprehensive and accurate portrayal of its functional role in this context.

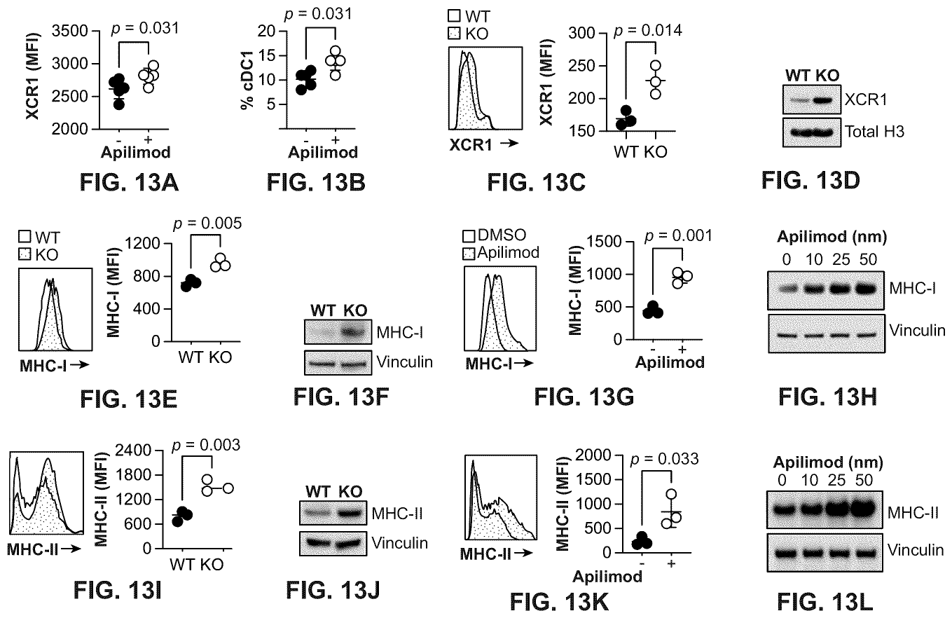

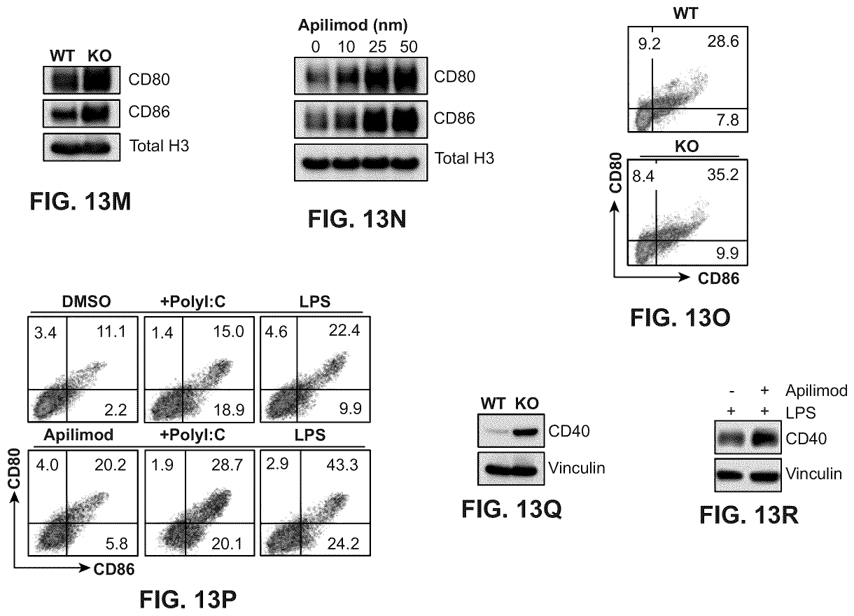

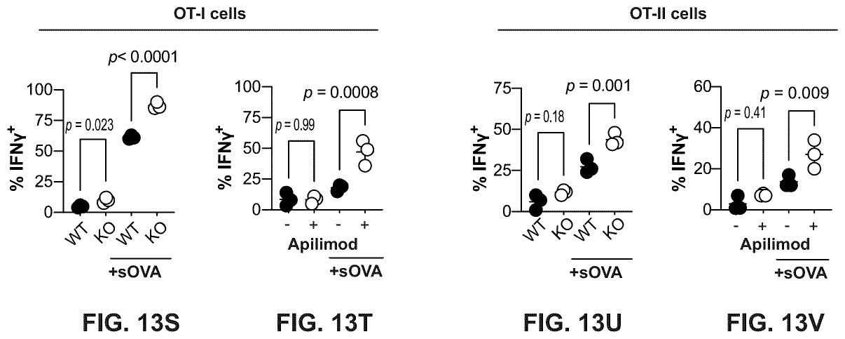

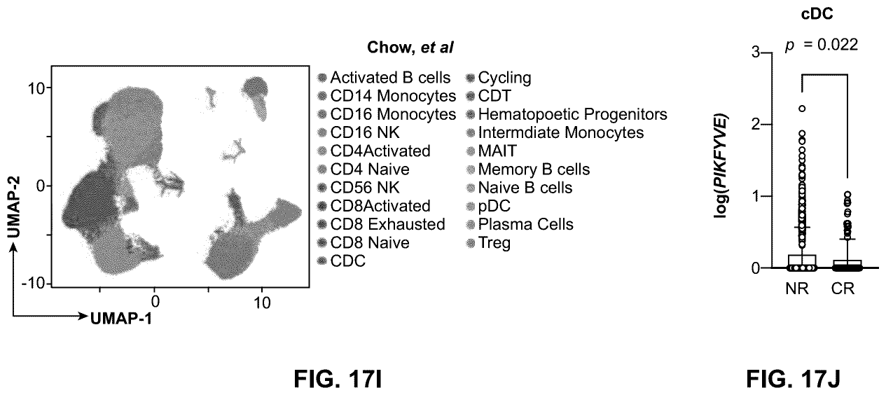

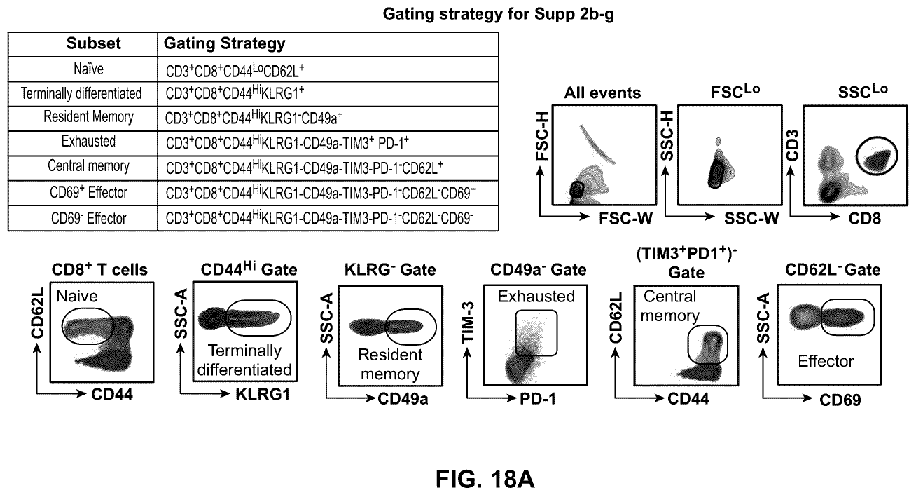

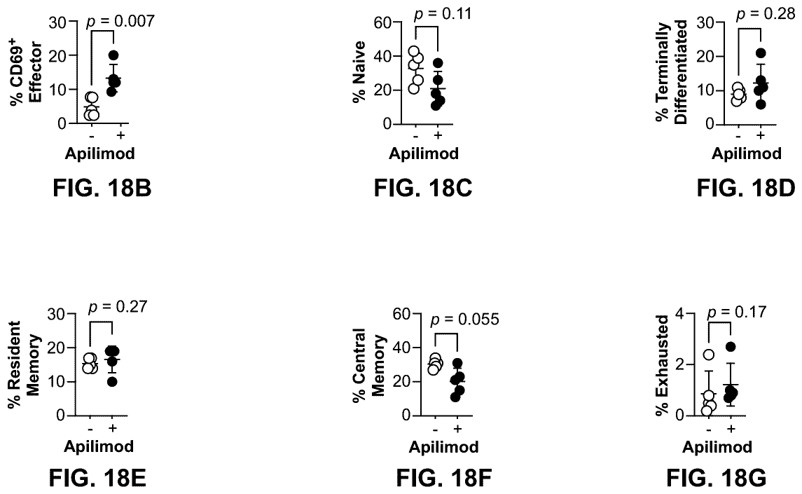

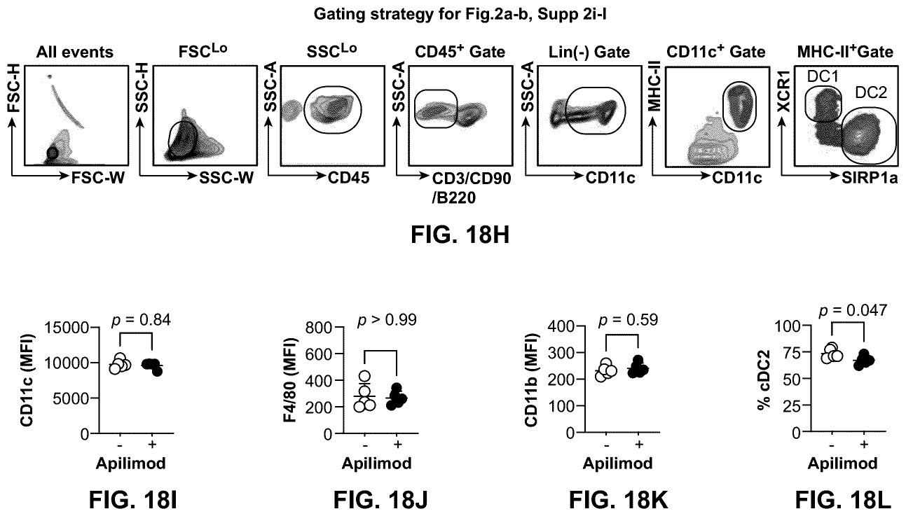

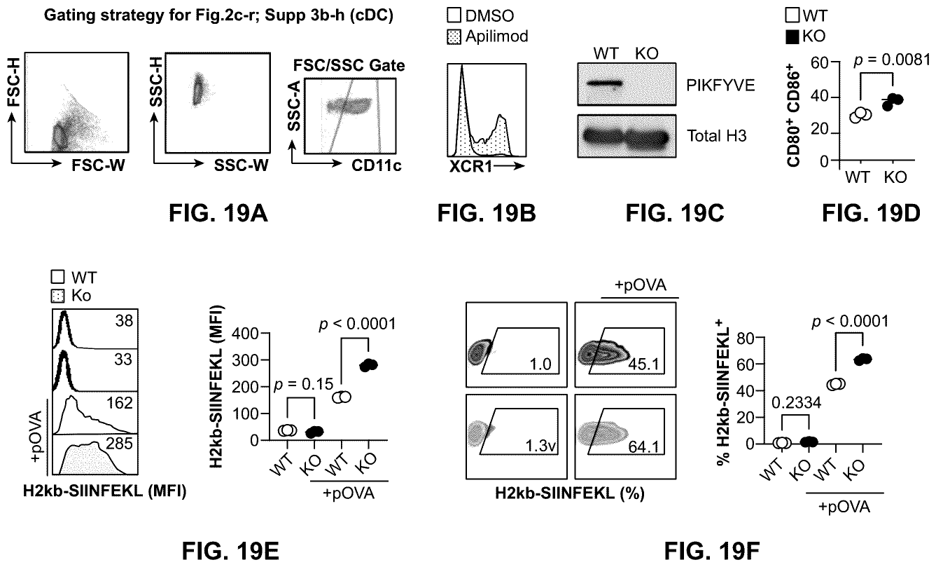

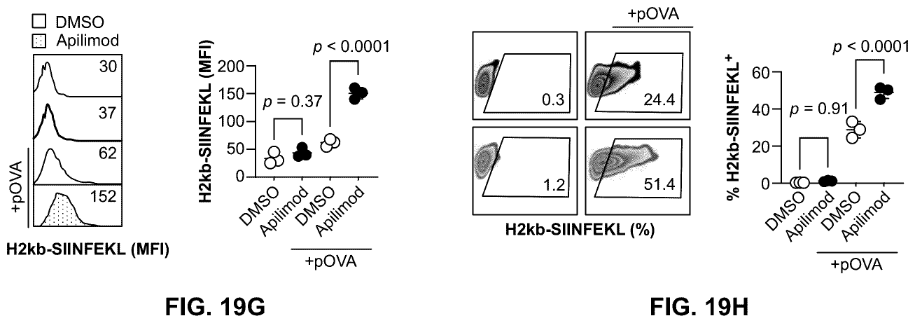

First, non-tumor-bearing, wild-type mice were treated with apilimod, a potent and specific PIKfyve inhibitor that has been studied in many cell types and evaluated in Phase II clinical trials See, e.g., Qiao, Y. et al., Autophagy Inhibition by Targeting PIKfyve Potentiates Response to Immune Checkpoint Blockade in Prostate Cancer. Nat Cancer 2021:2: 978-993; Choy, C. H. et al., J. Cell Sci. 2018:131; Hessvik, N. P. et al., Cell. Mol. Life Sci. 2016:73: 4717-4737; Baranov, M. V. et al., iScience 2019:11: 160-177; Sharma, G. et al., Autophagy 2019:15: 1694-1718. Cai, X. et al., Chem. Biol. 2013:20: 912-921; Magid Diefenbach, C. S. et al., J. Clin. Oncol. 2020:38: 8017-8017; Wada, Y. et al., PLoS One 2012:7; Krausz, S. et al., Brief Arthritis Rheum. 2012:64: 1750-1755; Kang, Y.-L. et al., Proc. Natl. Acad. Sci. U.S.A 2020:117: 20803-20813; Gayle, S. et al., Blood 2017: 1768-1778. Next, a global assessment of immune cells enriched from spleens was performed. It was discovered that increased proportions of CD69⁺CD8⁺ T cells in apilimod-treated mice compared to vehicle (FIG. 18A,B). There were no changes in the percentage of terminally differentiated, memory or exhausted CD8⁺ T cells between groups (FIG. 18C-G). Myeloid lineages (FIG. 18H) were also assessed and it was observed that their was no change in the total surface expression of CD11c, F4/80, or CD11b (FIG. 18I-K). Interestingly, there was an increase in the total surface expression of XCR1 with apilimod treatment (FIG. 13A). Within the cDC subset, there was an increase in the relative percentage of cDC1s (FIG. 13A) versus cDC2 cells (FIG. 18L) with apilimod treatment. This warranted further study as cDC1s are a cDC subtype which expresses XCR1 and is involved in cross-presentation and CD8⁺ T cell-mediated anti-tumor responses See, e.g., Hildner, K. et al., Science 2008:322: 1097-1100; Durai, V. et al., Nat. Immunol. 2019:20: 1161-1173; Ferris, S. T. et al., Nature 2020:584: 624-629. An increase in total surface expression of XCR1 was also observed in cultured cDCs treated with apilimod compared to dimethyl sulfoxide (DMSO) (FIG. 19A,B). To confirm that these changes were due to loss of functional PIKfyve, Pikfyve^{f/f}(“WT”) mice with Itgax^{Tg/0} (“CD11c-Cre”) mice were bred to specifically and conditionally delete PIKfyve in CD11c⁺ DCs (“KO”). cDCs were cultured and isolated from WT and KO mice in vitro and validated loss of PIKfyve expression (FIG. 19C). Intriguingly, it was observed that surface (FIG. 13C) and total protein (FIG. 13D) expression of XCR1 also increased in cDCs from PIKfyve KO versus WT mice.

Given this evidence that PIKfyve loss could alter cDC state, phenotypic changes in cDCs with PIKfyve ablation were assessed broadly. It was found that surface (FIG. 13E) and total protein (FIG. 13F) expression of MHC-I (H2-kb, H2-kd) were significantly increased in KO cDCs when compared to those from WT mice. To determine if these findings were phenocopied by pharmacologic PIKfyve inhibition, cDCs from WT mice were treated with apilimod. Apilimod-treated cDCs showed increased surface and total protein expression of MHC-I when compared to control (FIG. 13G,H). Similarly, surface (FIG. 13I) and total protein expression (FIG. 13J) of MHC-II were increased in the KO versus WT cDCs. Furthermore, apilimod-treated cDCs had increased surface and total protein expression of MHC-II (FIG. 13K,L).

To understand whether this increase in MHCI/II expression occurred in isolation or represented a change in overall DC maturation, the expression of co-stimulatory molecules was examined. Total protein levels of CD80 and CD86 expression were increased in PIKfyve KO versus WT cDCs (FIG. 13M). Apilimod treatment induced CD80 and CD86 expression in WT DCs in a dose-dependent manner (FIG. 13N). The percentage of CD80⁺CD86⁺ cDCs was also increased with PIKfyve KO (FIG. 13O, FIG. 19D) compared to WT. This increase in CD80⁺CD86⁺ cDCs was mirrored in apilimod-treated cells alone and in combination with PolyI:C and LPS (FIG. 13P). Furthermore, the total expression of CD40 was observed to increase in PIKfyve KO versus WT cDCs (FIG. 13Q). Addition of apilimod to LPS treatment also increased total expression of CD40 when compared to DMSO-treated cDCs (FIG. 13R).

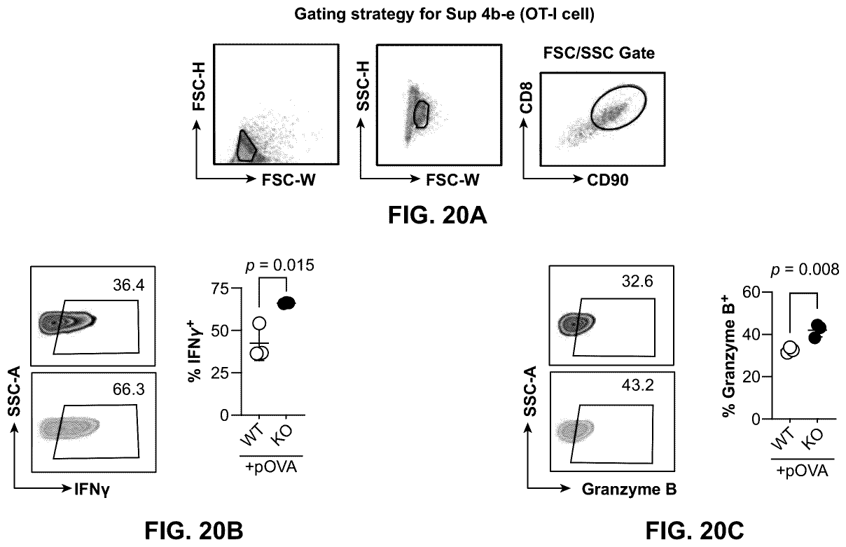

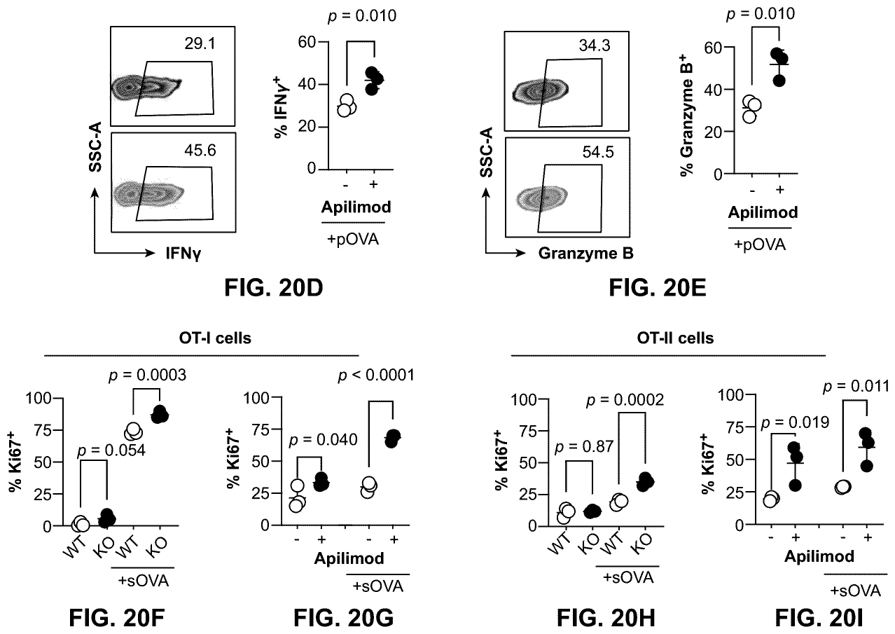

As genetic and pharmacologic ablation of PIKfyve affected MHC and costimulatory molecules, it was hypothesized that PIKfyve could, consequently, alter the ability of cDCs to present antigens and subsequently activate antigen-specific T cells. To test this possibility, ovalbumin (OVA) and OT-I/OT-II cell systems were utilized See, e.g., Yu, J. et al., Nat. Med. 2021:27: 152-164; Hogquist, K. A. et al., Cell 1994:76: 17-27; Lin, H. et al., Cancer Cell 2021:39: 480-493. When cultured with SIINFEKL peptide (pOVA), PIKfyve KO cDCs had greater total intensity and percentage of cells with H2-kb-SIINFEKL surface expression compared to WT cDCs (FIG. 19E,F). Similarly, apilimod-treated cDCs had greater total intensity and percentage of cells with H2-kb-SIINFEKL surface expression on cDCs compared to DMSO-treated cDCs (FIG. 19G,H). In addition, it was observed that peptide antigen-loaded PIKfyve KO cDCs induced a greater percentage of IFNγ⁺ and granzyme B⁺ OT-I cells compared to WT (FIG. 20A-C). This increase in T cell activation was also seen with apilimod treatment compared to DMSO (FIG. 20D,E). Finally, cDCs were loaded with soluble OVA protein (sOVA) which were then co-cultured with OT-I and OT-II cells. There were higher percentages of Ki67⁺ (FIG. 20F) and IFNγ⁺ (FIG. 13S) OT-I cells following co-culture with sOVA-loaded PIKfyve KO cDCs compared to WT cDCs. These increases were also demonstrated in OT-I cells co-cultured with apilimod-treated cDCs compared to DMSO (FIG. 13T, FIG. 20G). In addition, there were higher percentages of Ki67⁺ and IFNγ⁺ OT-II cells following co-culture with PIKfyve KO and apilimod-treated cDCs when compared to their respective controls (FIG. 20H,I, FIG. 13U,V). Together, these data indicate that PIKfyve alters DC state and negatively controls DC maturation and function.

### Example 8

### PIKfyve Suppresses NF-κB Activation in DCs

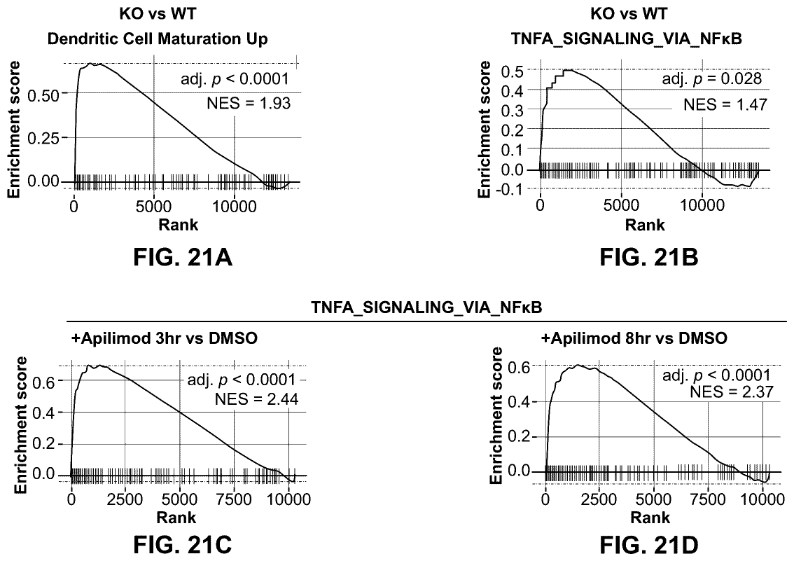

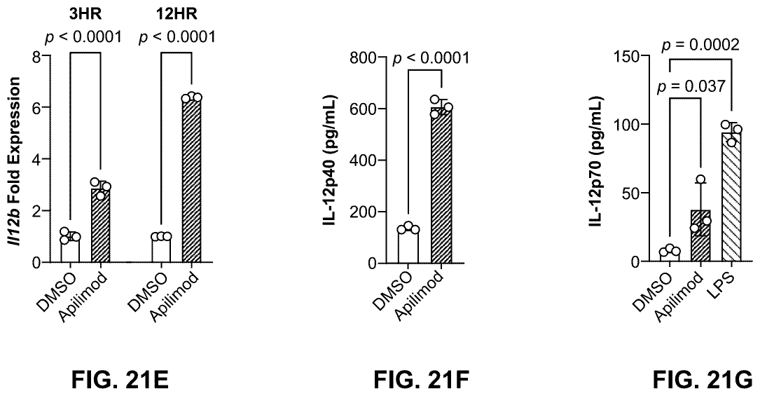

To explore the mechanism through which PIKfyve suppresses DCs, RNA-seq studies were conducted. First, gene set enrichment analysis was performed on genes differentially expressed between PIKfyve KO versus WT cDCs. Review of MSigDB curated gene sets (“C2”) revealed that a gene signature of enhanced DC maturation was positively enriched in PIKfyve KO cDCs (FIG. 21A) consistent with the phenotypic and functional characterization of these cells.

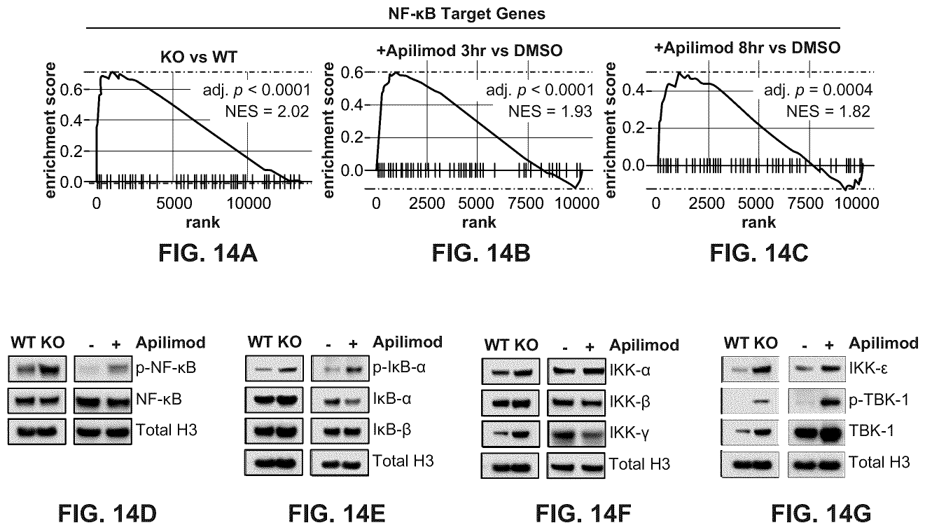

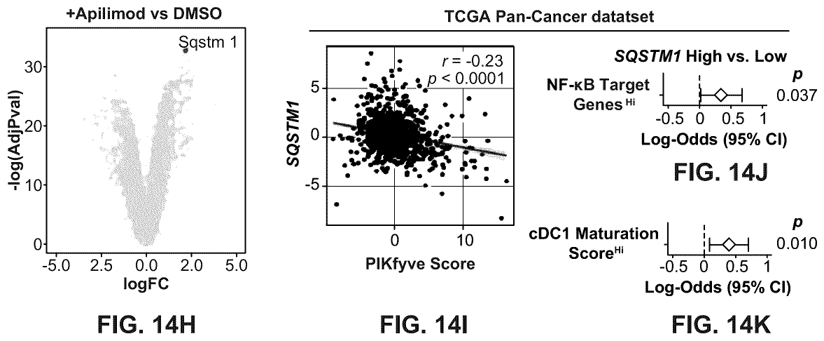

Furthermore, examination of MSigDB hallmark gene sets (“H”) revealed positive enrichment of the “TNF_SIGNALING_VIA_NFκB” gene set in PIKfyve KO versus WT cDCs (FIG. 21B). This was intriguing as NF-κB is known to be an essential transcription factor for driving the overall maturation and acute activation of DCs See, e.g., Ouaaz, F. et al., Immunity 2002:16: 257-270; Ghislat, G. et al., Sci. Immunol. 2021:6; Wang, J. et al., J. Immunol. 2007:178: 6777-6788; Baratin, M. et al., Immunity 2015:42: 627-639. Given these findings, it was hypothesized that a more general signature of NF-κB downstream gene targets would be affected in PIKfyve KO cDCs. Thus, a list of validated NF-κB gene targets from commercial assays was curated. In a posteriori analysis, it was observed that this signature representing direct activation of downstream NF-κB genes was positively enriched in PIKfyveKO versus WT cDCs (FIG. 14A).

To corroborate these data suggesting NF-κB regulation by PIKfyve, WT cDCs were treated with apilimod to identify differentially expressed genes at time points coinciding with early DC activation. Examination of MSigDB hallmark gene sets revealed positive enrichment of “TNF_SIGNALING_VIA_NFκB” in apilimod-treated cDCs at 3 and 8 hours when compared to DMSO (FIG. 21C,D). Importantly, direct NF-κB gene targets were positively enriched in apilimod-treated cDCs at these early time points, thus substantiating the findings from the genetic model (FIG. 14B,C). Interestingly, apilimod treatment increased Il12b transcripts (FIG. 21E) in addition to the secretion of IL-12p40 (FIG. 21F) and IL-12p70 (FIG. 21G). Finally, both genetic loss and pharmacological inhibition of PIKfyve increased relative protein levels of p-NF-κB to NF-κB was confirmed (FIG. 14D).

NF-κB is regulated by dynamic, cascading changes in a well-characterized system of upstream cytosolic regulatory proteins that culminate in an altered transcriptional landscape See, e.g., Zhang, Q. et al., Cell 2017:168: 37-57; Yum, S. et al., Proc. Natl. Acad. Sci. U.S.A. 2021:118. Interestingly, neither genetic nor pharmacological PIKfyve ablation changed levels of IκB-β, but PIKfyve loss or inhibition did increase levels of p-IκB-α relative to IκB-α, which would allow for increased NF-κB phosphorylation and activation (FIG. 14E). To understand if this regulation extended further upstream, the canonical and alternate/non-canonical regulators of the IκB kinase complex were also investigated. The abundance of IKK-α and IKK-β were observed to be unchanged with PIKfyve ablation (FIG. 14F). Interestingly, the abundance of IKK-ε, p-TBK-1, and TBK-1 were found to increase with genetic and pharmacological PIKfyve ablation (FIG. 14G).

These novel findings motivated further exploration of how PIKfyve may alter NF-κB activation. Intriguingly, Sqstm1 emerged as the most significantly upregulated gene in apilimod-treated cDCs when compared to DMSO (FIG. 14H). Sqstm1 was also shown to be differentially expressed in KO versus WT cDCs. Sqstm1 is a promising mechanistic candidate in PIKfyve regulation as it is involved in vesicular trafficking, autophagy and lysosome and proteasome degradation. See, e.g., Zhou, B. et al., Nat. Microbiol. 2020:5: 1576-1587; Aflaki, E. et al., Aging Cell 2016:15: 77-88. Importantly, Sqstm1 can regulate NF-κB activation under various conditions and is known to interact with TBK-1 See, e.g., Zhou, B. et al., Nat. Microbiol. 2020:5: 1576-1587; Aflaki, E. et al., Aging Cell 2016:15: 77-88; Schlütermann, D. et al., Sci. Rep. 2021:11: 13863; Matsumoto, G. et al., Hum. Mol. Genet. 2015:24: 4429-4442; Prabakaran, T. et al., EMBO J. 2018:37; Zhong, Z. et al., Cell 2016:164: 896-910; Lobb, I. T. et al., Mol. Cancer Res. 2021:19: 274-287; Shi, J. et al., Autophagy 2013:9: 1591-1603; Schwob, A. et al., Sci. Rep. 2019:9: 16014.

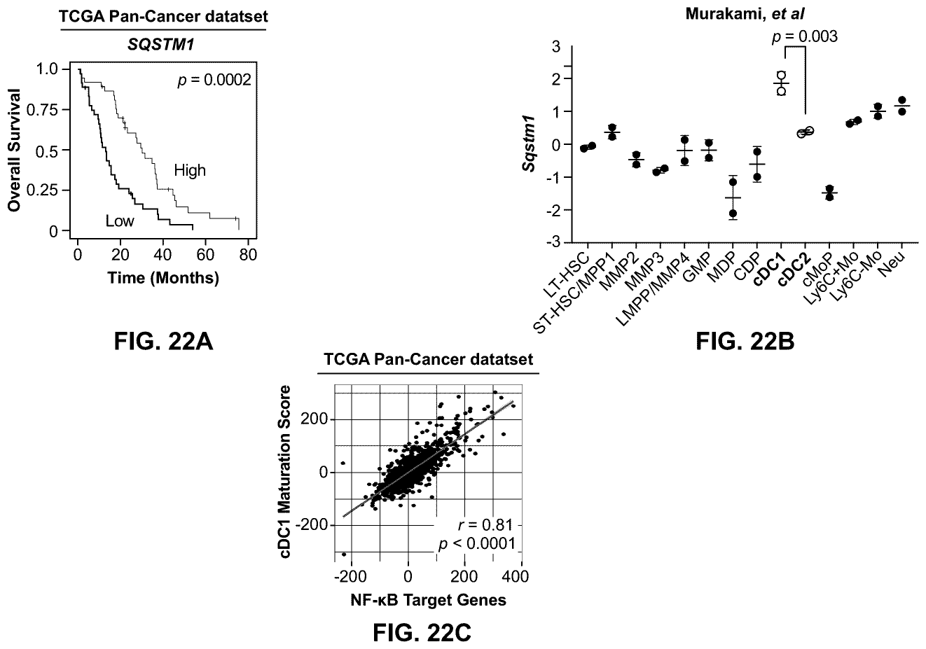

Utilizing the TCGA Pan-Cancer data, it was found that high SQSTM1 levels were associated with better overall survival in patients (FIG. 22A). It was also observed that high SQSTM1 negatively correlated with PIKfyve scores (FIG. 14I). Furthermore, patients with high NF-κB gene target scores were more likely to have high SQSTM1 expression (FIG. 14J). Recent research has demonstrated that NF-κB pathway activation may direct maturation states within the mouse cDC1 subset. See, e.g., Ghislat, G. et al., Sci. Immunol. 2021:6. It was discovered that Sqstm1 expression was higher in mouse cDC1 when compared to most other myeloid lineages, including cDC2 (FIG. 22B). See, e.g., Murakami, K. et al., Nat. Immunol. 2021:22: 301-311. This was further investigated within the patient data. As expected, published research shows that “cDC1 maturation score” positively correlated with NF-κB gene targets in TCGA Pan-Cancer patient samples (FIG. 22C). See, e.g., Ghislat, G. et al., Sci. Immunol. 2021:6. Moreover, patients who had higher cDC1 maturation scores were more likely to have high SQSTM1 expression (FIG. 14K). Collectively, these data suggest that PIKfyve mediates suppression of DC transcriptional maturation programs through the alternate/non-canonical NF-κB regulatory pathway.

### Example 9

### PIKfyve Loss in DCs Enhances Anti-Tumor Immunity In Vivo

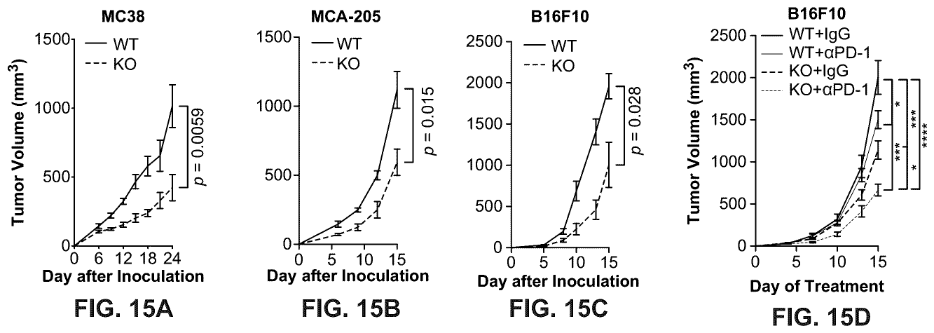

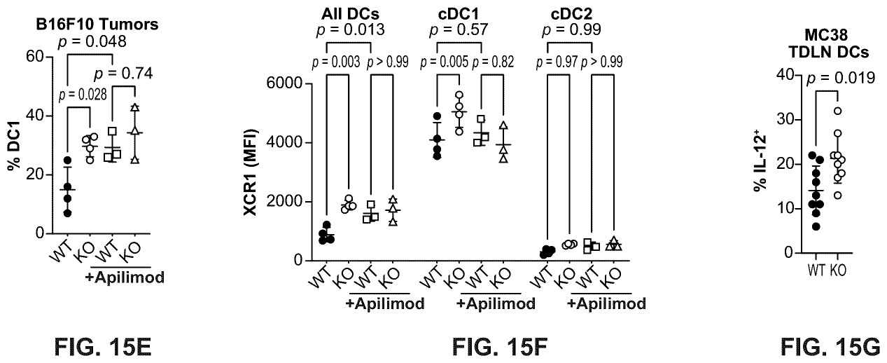

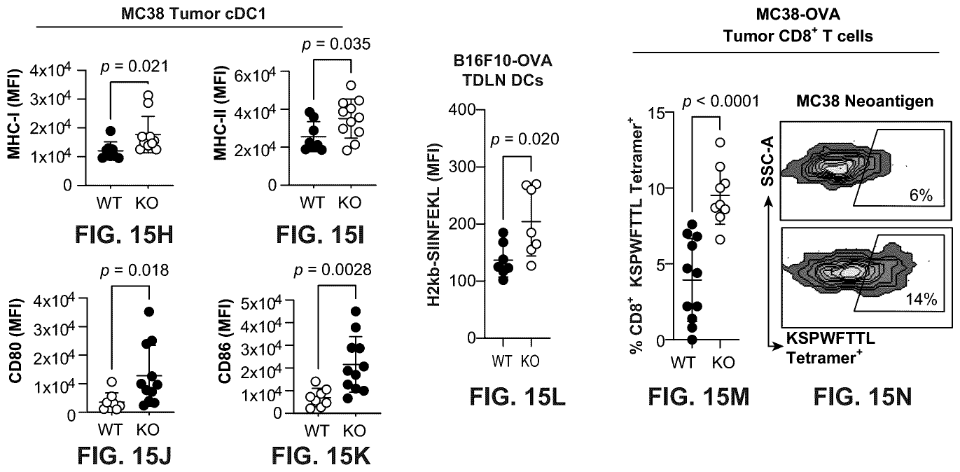

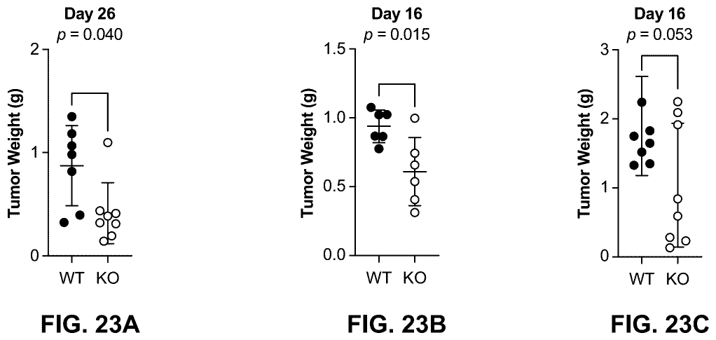

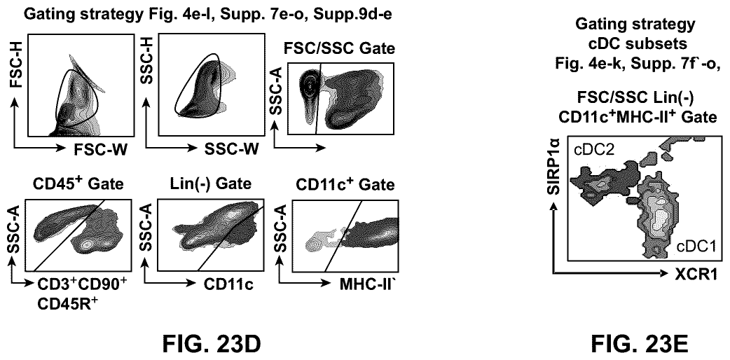

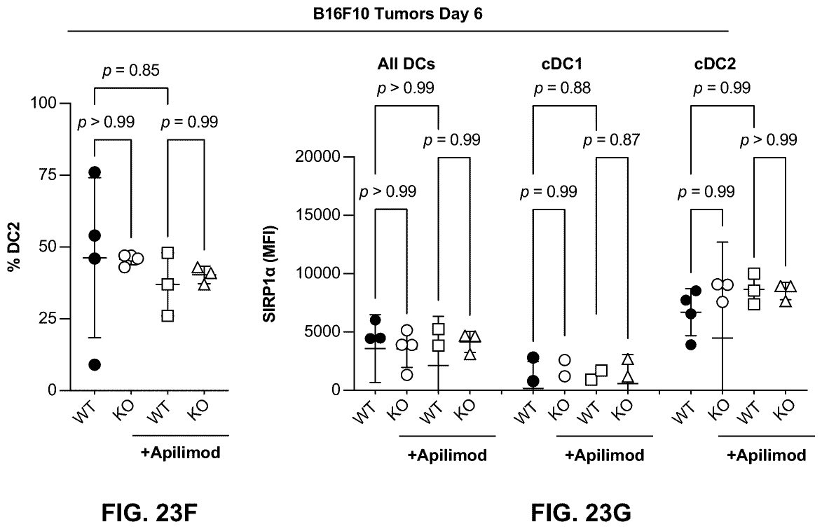

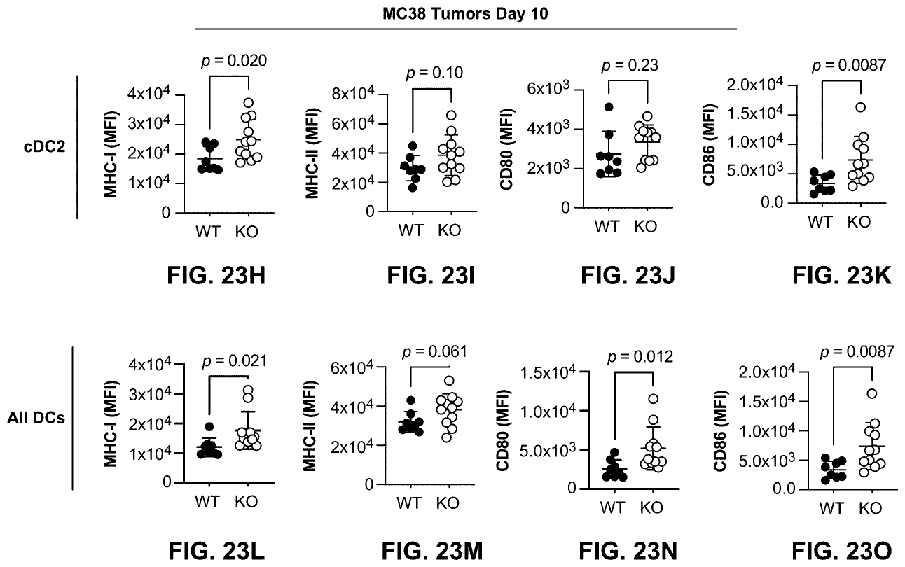

Since the above results showed that PIKfyve could fundamentally transform cDCs, it was examined whether these alterations had any effect on a disease setting. It was hypothesized that the loss of PIKfyve in DCs could attenuate tumor growth in syngeneic mouse models of cancer. Subcutaneous MC38 tumors injected into PIKfyve KO mice manifested reduced tumor growth (FIG. 15A) and tumor weights (FIG. 23A) compared to WT mice. Furthermore, this inhibitory effect on tumor growth and terminal tumor weights was also seen in KO mice bearing MCA-205 (FIG. 15B, FIG. 23B) and B16F10 (FIG. 15C, FIG. 23C) tumors. Additionally, the loss of PIKfyve in DCs could alter response to anti-PD-1 therapy in the ICB-resistant B16F10 model was explored. Tumor growth inhibition with anti-PD-1 therapy was enhanced when comparing endpoint tumor volume in PIKfyve KO mice to wild type mice (FIG. 15D).

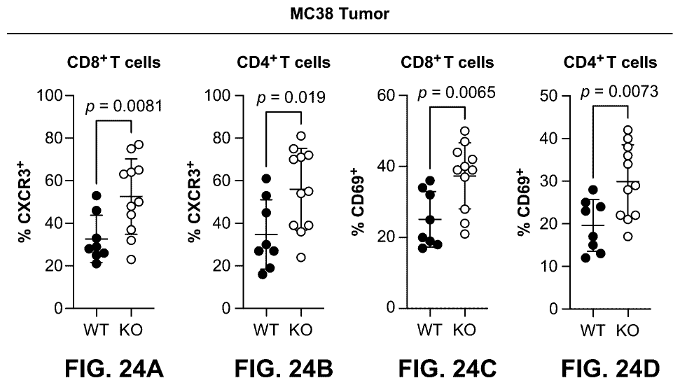

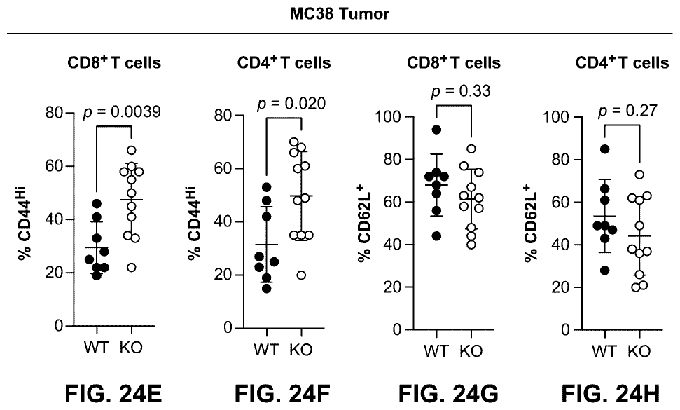

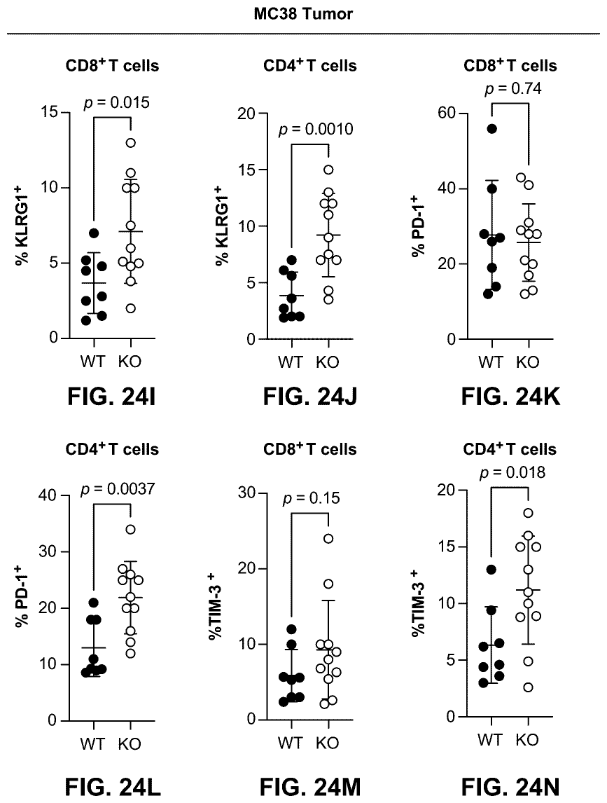

The effects of PIKfyve inhibitor treatment on tumor infiltrating DCs and T cells isolated from DC-selective PIKfyve KO versus WT mice in multiple tumor models was then assessed. There was an increase in the percentage of cDC1s in KO versus WT mice in B16F10 tumors (FIG. 15E, FIG. 23D). Importantly, treatment with apilimod increased the percentage of cDC1s amongst the WT mice but resulted in no additional increase in the KO mice. Notably, these differences were the same when evaluating total surface XCR1 expression between groups (FIG. 15F). There were no changes in the percentage of cDC2s (FIG. 23E,F) or total surface SIRP1α expression (FIG. 23G) between any groups. Furthermore, it was observed that an increased proportion of IL-12-expressing DCs in MC38 tumors from PIKfyve KO versus WT mice (FIG. 15G). When comparing PIKfyve KO versus WT tumors, MHC-I and cDC maturation markers (MHC-II, CD80, and CD86) were significantly increased in the cDC1 subset (FIG. 15H-K) when compared to cDC2 subset (FIG. 23H-K) or all DCs (FIG. 23L-O). The tumor infiltrating potential of CD8⁺ and CD4⁺ T cells from MC38 tumors was also assessed. In the PIKfyve KO mice, there was a higher percentage of CXCR3⁺ (FIG. 24A,B) and CD69⁺ (FIG. 24C,D) effector T cells and a higher percentage of CD44^{H}i effector memory T cells (FIG. 24E,F) without changes in CD62L⁺ naïve T cells (FIG. 24G,H). Interestingly, though there was a higher proportion of KLRG1⁺ (FIG. 24I,J) CD8⁺ and CD4⁺ T cells in the KO group, PD-1 (FIG. 24K,L) and TIM-3 (FIG. 24M,N) were only increased in the CD4⁺ T cells. Hence, these data showed that PIKfyve loss remodeled DC phenotype and T cell function in tumors in vivo.

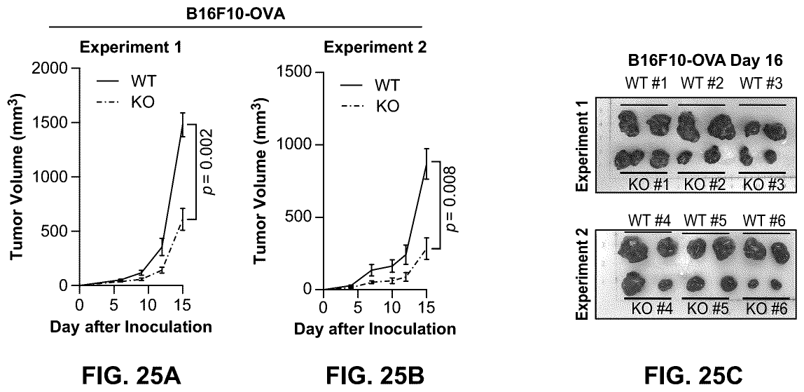

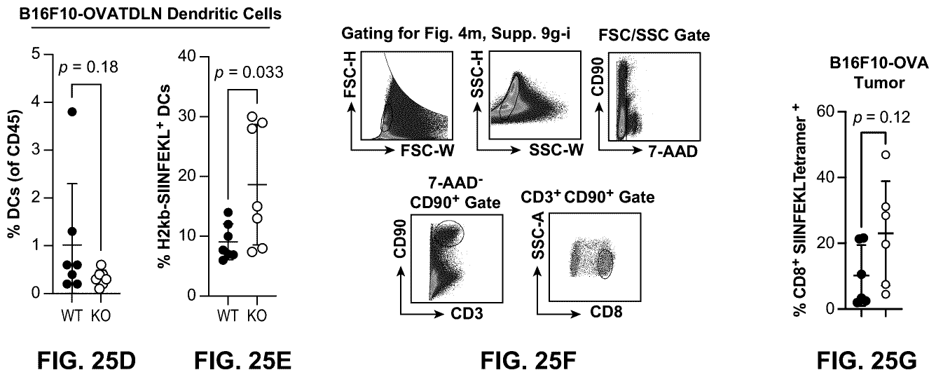

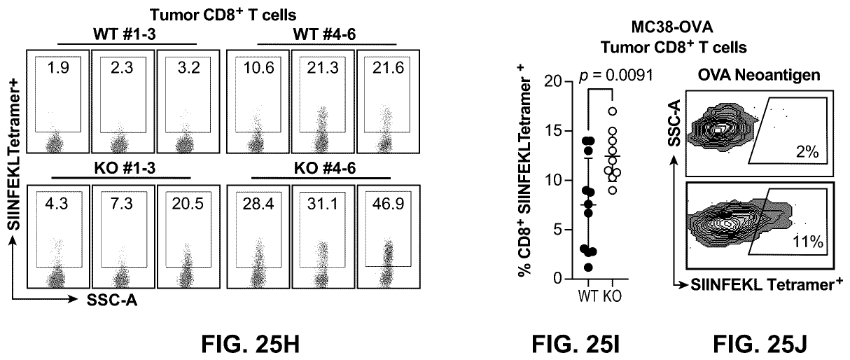

Additionally, it was investigated whether the immune response was altered with PIKfyve loss in DCs. As expected, the growth of B16F10-OVA tumors (FIG. 25A-C) was decreased when inoculated in KO versus WT mice. No difference was observed in the percentage of CD11c⁺MHC-II⁺ DCs isolated from tumor-draining lymph nodes (TDLNs) compared to all CD45⁺ cells (FIG. 25D). Importantly, overall H2-kb-SIINFEKL expression on the surface of DCs from the KO mice was higher than those from the WT (FIG. 15L). Concordantly, the percentage of H2kb-SIINFEKL⁺ DCs was increased in the KO mice (FIG. 25E). When comparing the littermate pairs of WT and KO mice, only a marginal increase in the percentage of OVA antigen-specific (SIINFEKL) tetramer⁺ CD8⁺ T cells isolated from B16F10-OVA tumors from each pair (FIG. 25F-H). In the MC38-OVA model, there was a higher percentage of SIINFEKL tetramer⁺ CD8⁺ T cells isolated from tumors (FIG. 25I,J). MC38-OVA tumor had a higher proportion of MC38 tumor neoantigen-specific (KSPWFTTL) tetramer⁺ CD8⁺ T cells (FIG. 15M,N) demonstrating that PIKfyve loss enhanced DC-mediated antigen presentation and priming of antigen-specific CD8⁺ T cells in an in vivo tumor model. Combined, these data show that PIKfyve in DCs can modulate tumor outcomes in vivo.

### Example 10

### Immune Effects of PIKfyve are DC-Dependent

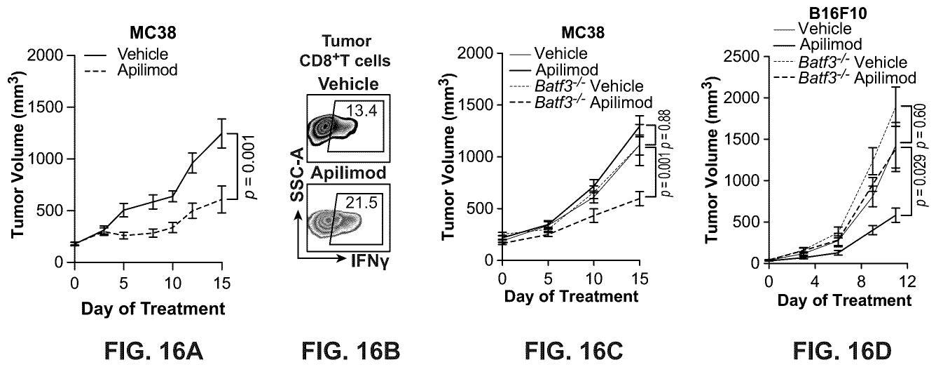

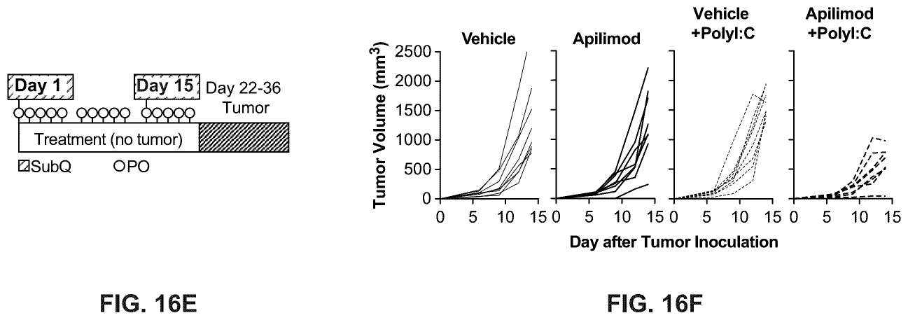

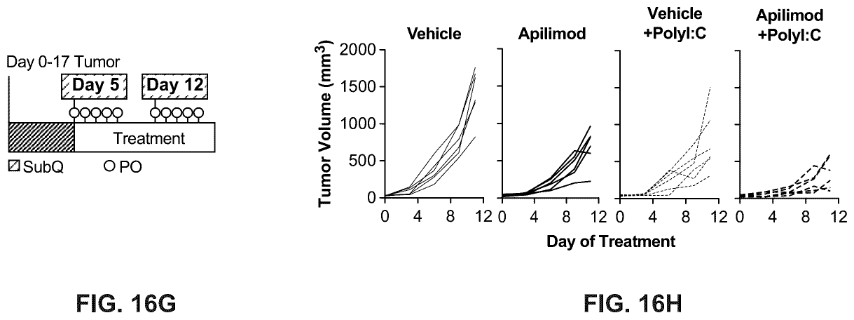

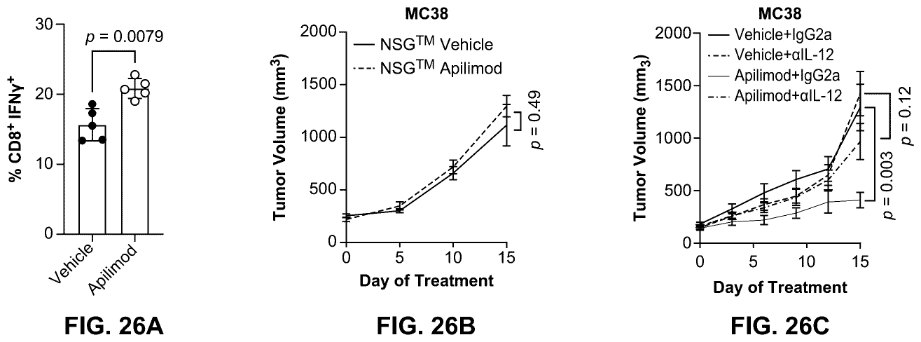

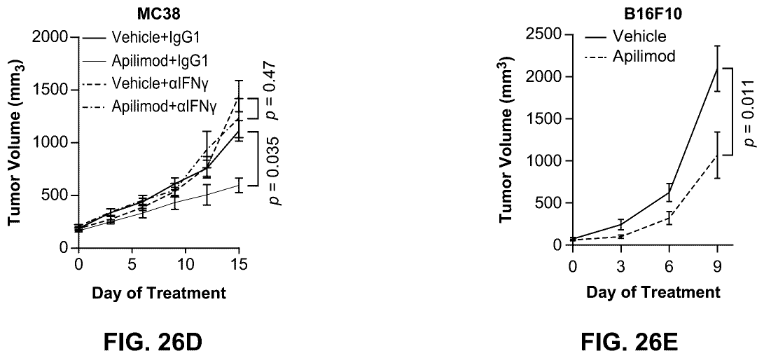

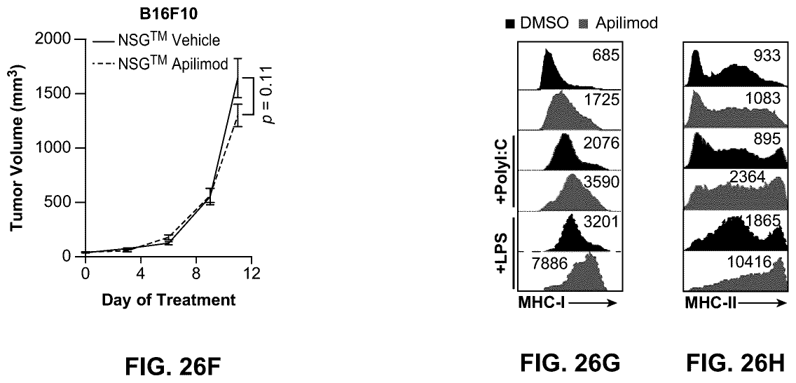

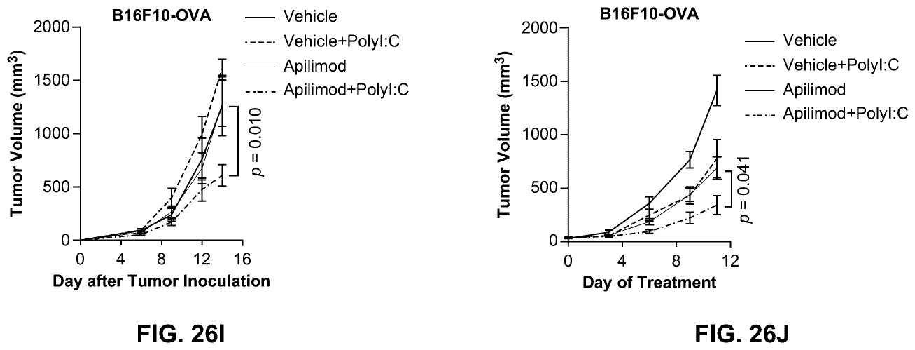

Though PIKfyve inhibitors have shown anti-tumor effects in various preclinical cancer models, it is unclear if the activity of DCs directly contributes to their therapeutic effect. See, e.g., Qiao, Y. et al., Nat. Cancer 2021:2: 978-993; Magid Diefenbach, C. S. et al., J. Clin. Oncol. 2020:38: 8017-8017; Gayle, S. et al., Blood 2017:129: 1768-1778; Kim, S. M. et al., J. Clin. Invest. 2016:126: 4088-4102; Hou, J.-Z. et al., Oncol. Rep. 2019:41: 1971-1979; O'Connell, C. E. & Vassilev, A. Cancer Res. 2021:81: 2903-2917 (2021). The combined genetic and pharmagologic in vivo experiments showed no additional change in PIKfyve KO DCs with the addition of PIKfyve inhibitor treatment. Therefore, it was further hypothesized that therapeutic PIKfyve inhibition required the presence of DCs to exert its full anti-tumor effect in vivo. To this end, WT mice were inoculated with subcutaneous MC38 tumors. In vivo, of the WT mice, treatment with apilimod reduced MC38 tumor growth compared to vehicle (FIG. 16A) and increased the percentage of intratumoral IFNγ⁺CD8⁺ T cells (FIG. 16A, FIG. 26A). The efficacy of apilimod was lost in immune-deficient NSG mice (FIG. 26A). To test whether drug efficacy required DCs, Batf3^{-/-} mice, which have loss of cDC1s, were used See, e.g., Hildner, K. et al., Science 2008:322: 1097-1100; Durai, V. et al., Nat. Immunol. 2019:20: 1161-1173; Ferris, S. T. et al., Nature 2020:584: 624-629. Importantly, loss of apilimod efficacy remained when comparing Batf3^{-/-} mice to immune-competent, WT mice (FIG. 16C).

The importance of functional DC and T cell signaling pathways in apilimod treatment efficacy was next investigated. In MC38 tumor-bearing mice, it was observed that the efficacy of apilimod was reduced when mice were treated with neutralizing monoclonal antibodies against IL-12 (FIG. 26C) or IFNγ (FIG. 26D) compared to isotype controls See, e.g., Garris, C. S. et al., Immunity 2018:49: 1148-1161; Qiao, Y. et al., Nat. Cancer 2021:2: 978-993; Wang, W. et al., Nature 2019:569: 270-274. As IL-12 is often expressed by DCs and IFNγ by T cells, the data suggests that in vivo efficacy of PIKfyve inhibition requires the presence of functional DC signaling and intact DC and T cell signaling pathways. To further validate this possibility in an additional tumor model, WT, non-transgenic mice were inoculated with subcutaneous B16F10 tumors. Treatment with apilimod in vivo reduced tumor growth compared to vehicle in this model (FIG. 26E). Again, efficacy of apilimod was lost when B16F10 tumors were inoculated into NSG (FIG. 26F) or Batf3^{-/-} mice (FIG. 16D).

As vaccines are a DC-dependent immunotherapy strategy, it was explored whether apilimod, as a DC-modulating and DC-dependent agent could potentially modulate cancer vaccine strategies. PolyI:C and TLR agonists are commonly used as vaccine adjuvants in different tumor vaccine models See, e.g., Martins, K. A. O., Bavari, S. and Salazar, A. M. Expert Rev. Vaccines 2015:14: 447-459. DCs were treated with PolyI:C or lipopolysaccharide (LPS) in the presence of apilimod in vitro. It was found that PolyI:C, LPS, or apilimod stimulated MHC-I and MHC-II expression, and apilimod further enhanced this effect (FIG. 26G,H). In a proof-of-concept experiment, it was explored whether PIKfyve inhibition, as a DC-maturing and DC-dependent therapy, could potentiate the effects of PolyI:C against tumor growth in vivo. First, mice were treated with apilimod or vehicle in addition to subcutaneous PolyI:C or water (control) for 21 days prior to inoculation with B16F10-OVA tumors (FIG. 16E). The combination of pre-treatment with apilimod and PolyI:C was observed to decrease subsequent tumor growth compared to control or either agent alone (FIG. 16F, FIG. 26I).

Mice were then inoculated with B16F10-OVA tumors first and followed by combination therapy with vehicle or apilimod in addition to PolyI:C or water (FIG. 16G). Apilimod and PolyI:C alone resulted in modest tumor growth inhibition which was further potentiated by combination with both agents (FIG. 16H, FIG. 26J). These data suggest that PIKfyve inhibitors are viable DC-dependent drugs and are promising candidates for novel combination therapy strategies with human-relevant DC-stimulating agents and adjuvants in cancer and other diseases.

### Example 11

### Discussion

It was explored whether therapeutically actionable molecular signaling pathways regulate DCs. Experiments revealed a previously unknown fundamental mechanism for PIKfyve in controlling DC state, maturation, and function through NF-κB regulation and demonstrated its ability to potentiate immunotherapy strategies through DCs. Thus, generating key translational insight into the potential of PIKfyve inhibitors for enhancing DC-dependent therapies in cancer, such as ICB and vaccines.

These data characterize a clinically viable protein kinase inhibitor strategy to enhance DC function. Though the investment into these cancer-targeted drugs has transformed cancer therapy outcomes for many patients, it is clear that further insight is required to understand how to achieve universally durable and curative responses. See, e.g., Cohen, P. et al., Nat. Rev. Drug Discov. 2021:20: 551-569; Arora, A. and Scholar, E. M. J. Pharmacol. Exp. Ther. 2005:315: 971-979; Madhusudan, S. and Ganesan, T. S. Clin. Biochem. 2004:37: 618-635; Zhong, L. et al., Signal Transduct Target Ther. 2021:6: 201. In view of the essential role of immunity in cancer, studies have shown that these drugs may modulate CD8⁺ T cell responses and the efficacy of immunotherapy See, e.g., Wang, H. et al., Sci. Adv. 2021:7; Hu-Lieskovan, S. et al., Sci. Transl. Med. 2015:7: 279; Baumann, D. et al., Nat. Commun. 2020:11: 2176; Peng, D. H. et al., Nat. Commun. 2021:12: 2606; Gong, K. et al., Nat. Cancer 2020:1: 394-409; Qiao, Y. et al., Nat. Cancer 2021:2: 978-993; Ribas, A. et al., Nat. Commun. 2020:11: 6262; Shen, C. I. et al., Sci. Rep. 2021:11: 16122; O'Shea, P. J. et al., J. Clin. Orthod. 2021:39: 2030-2030; Sugiyama, E. et al., Sci. Immunol. 2020:5. Notably, their impact on DCs remains largely unexplored. DCs have long been a desired target for cancer therapy given their essential role in T cell immunity. See, e.g., Wculek, S. K. et al., Nat. Rev. Immunol. 2020:20: 7-24; Domogalla, M. P. et al., Front. Immunol. 2017:8: 1764; Joffre, O. P. et al., Nat. Rev. Immunol. 2012:12: 557-569. It is challenging to manipulate DCs directly due to their small numbers, diversity of roles across subsets, and lack of specific targets. Herein, PIKfyve is identified as a clinically-druggable molecular target in DCs. This further highlights the potential of targeting PIKfyve in DCs to improve T cell responses and immunotherapy.

The data described herein identify a previously unexplored mechanistic association between PIKfyve, a lipid kinase that synthesizes phosphatidylinositol and regulates autophagy, and NF-κB, a transcription factor that is functionally critical for DC activation and maturation. See, e.g., Krishna, S. et al., Dev. Cell 2016:38: 536-547; Giridharan, S. S. P. et al., Elife 2022:11; Choy, C. H. et al., J. Cell Sci. 2018:131; Hessvik, N. P. et al., Cell. Mol. Life Sci. 2016:73: 4717-4737; Ouaaz, F. et al., Immunity 2002:16: 257-270; Ghislat, G. et al., Sci. Immunol. 2021:6; Wang, J. et al., J. Immunol. 2007:178: 6777-6788; Baratin, M. et al., Immunity 2015:42: 627-639; Zhang, Q. et al., Cell 2017:168: 37-57; Zolov, S. N. et al., Proc. Natl. Acad. Sci. U.S.A. 2012:109: 17472-17477. NF-κB signaling activation through PIKfyve ablation was observed to selectively occurr through the alternate or non-canonical regulators of the IκB inhibitor complex, such as TBK-1. See, e.g., Yum, S. et al., Proc. Natl. Acad. Sci. U.S.A. 2021:118; Xiao, Y. et al., J. Exp. Med. 2017:214: 1493-1507; Fitzgerald, K. A. et al., Nat. Immunol. 2003:4: 491-496. Furthermore, SQSTM1, which has been shown to regulate NF-κB signaling under a wide range of conditions, was identified as a possible mechanistic linchpin See, e.g., Zhou, B. et al., Nat Microbiol. 2020:5: 1576-1587; Aflaki, E. et al., Aging Cell 2016:15: 77-88; Lobb, I. T. et al., Mol. Cancer Res. 2021:19: 274-287; Shi, J. et al., Autophagy 2013:9: 1591-1603; Schwob, A. et al., Sci. Rep. 2019:9: 16014. As SQSTM1 is a critical component of autophagic regulation through TBK-1, vesicle trafficking, and transcriptional regulation, it is a potential nexus between PIKfyve and alternate or non-canonical NF-κB regulation. See, e.g., Zhou, B. et al., Nat Microbiol. 2020:5: 1576-1587; Aflaki, E. et al., Aging Cell 2016:15: 77-88; Lobb, I. T. et al., Mol. Cancer Res. 2021:19: 274-287; Schlutermann, D. et al., Sci. Rep. 2021:11: 13863; Matsumoto, G. et al., Hum. Mol. Genet. 2015:24: 4429-4442; Prabakaran, T. et al., EMBO J. 2018:37. In addition, it was found that PIKfyve may preferentially alter cDC1s in tumor models. These results are in line with a recent study demonstrating that NF-κB signaling may be crucial for cDC1 maturation. See, e.g., Ghislat, G. et al., Sci. Immunol. 2021:6. These findings provide a rationale for further investigation of the potential for PIKfyve to regulate DC identity and lineage determination.

Ultimately, these data provide a strategy to transcriptionally and functionally overcome DC suppression in the tumor microenvironment through a lipid kinase. Given that PIKfyve suppresses DC function, it can be reasoned that PIKfyve inhibitors may be selected to treat cancers with high PIKfyve expression in tumors and DCs, thereby effectively targeting both tumors and the immune system. Conversely, caution may be required when considering using PIKfyve inhibitors in cancers driven by NF-κB signaling See, e.g., Xia, Y., et al., Cancer Immunol. Res. 2014:2: 823-830. Thus, these fundamental insights into DC regulation may inform optimal, “precision medicine” treatment strategies for further evaluation of PIKfyve inhibitors in specific cancers.

These results represent the first to distinguish PIKfyve inhibition as a DC-dependent cancer therapy. These studies demonstrate that PIKfyve inhibitors target DCs directly, require DCs and DC/T cell signaling to prevent tumor progression, and potentiate ICB therapy. This DC-dependent nature of PIKfyve inhibitor efficacy is rooted in its ability to mediate antigen presentation to T cells. Thus, this data provides a mechanistic rationale for PIKfyve inhibitor therapy in combination with ICB and invites further inquiry into ways to potentiate other DC-dependent strategies.

In addition to ICB, tumor vaccination is an approach for cancer prevention and therapy. See, e.g., Romero, P. et al., Sci. Transl. Med. 2016:8: 334; Steinman, R. M. & Pope, M. J. Clin. Invest. 2002:109: 1519-1526; Melief, C. J. M. et al., Sci. Transl. Med. 2020:12; Tanyi, J. L. et al., Sci. Transl. Med. 2018:10; Carreno, B. M. et al., Science 2015:348: 803-808; Rosenblatt, J. et al., Sci. Transl. Med. 2016:8. Many vaccination strategies involve adjuvants that directly activate DCs. See, e.g., Martins, K. A. O. et al., Expert Rev. Vaccines 2015:14: 447-459; Carreno, B. M. et al., Science 2015:348: 803-808. In proof-of-principle experiments, it was found that PIKfyve inhibition could potentiate the anti-tumor effect of Poly I:C, a cancer vaccine adjuvant. As the results of cancer vaccine therapy have remained underwhelming, these data provides a rationale for further investigation of PIKfyve inhibitors to vaccinate against cancers and infectious diseases. See, e.g., Daud, A. I. The lancet oncology 2018:(19): 852-853. The results provided herein substantiate the broad potential of PIKfyve inhibition as a DC-enhancing strategy across disease states. See, e.g., Kang, Y. L. et al., Proc. Natl. Acad. Sci. U.S.A. 2020:117: 20803-20813; Huang, P. T. et al., Nat. Rev. Drug Discov. 2021:20: 730.

### REFERENCES

Sharma, P. and J. P. Allison, Immune checkpoint targeting in cancer therapy: toward combination strategies with curative potential. Cell, 2015. 161(2): p. 205-14.
Sharma, P., et al., The Next Decade of Immune Checkpoint Therapy. Cancer Discov, 2021. 11(4): p. 838-857.
Rosenberg, S. A. and N. P. Restifo, Adoptive cell transfer as personalized immunotherapy for human cancer. Science, 2015. 348(6230): p. 62-68.
Saxena, M., et al., Therapeutic cancer vaccines. Nat Rev Cancer, 2021. 21(6): p. 360-378.
Janjigian, Y. Y., et al., First-line nivolumab plus chemotherapy versus chemotherapy alone for advanced gastric, gastro-oesophageal junction, and oesophageal adenocarcinoma (CheckMate 649): a randomised, open-label, phase 3 trial. Lancet, 2021. 398(10294): p. 27-40.
Sahin, U., et al., An RNA vaccine drives immunity in checkpoint-inhibitor-treated melanoma. Nature, 2020. 585(7823): p. 107-112.
Larkin, J., et al., Combined Nivolumab and Ipilimumab or Monotherapy in Untreated Melanoma. N Engl J Med, 2015. 373(1): p. 23-34.
Borghaei, H., et al., Nivolumab versus Docetaxel in Advanced Nonsquamous Non-Small-Cell Lung Cancer. N Engl J Med, 2015. 373(17): p. 1627-39.
Matsushita, H., et al., Cancer exome analysis reveals a T-cell-dependent mechanism of cancer immunoediting. Nature, 2012. 482(7385): p. 400-4.
Robbins, P. F., et al., Mining exomic sequencing data to identify mutated antigens recognized by adoptively transferred tumor-reactive T cells. Nat Med, 2013. 19(6): p. 747-52.
Gubin, M. M., et al., Checkpoint blockade cancer immunotherapy targets tumour-specific mutant antigens. Nature, 2014. 515(7528): p. 577-81.
Waldman, A. D., J. M. Fritz, and M. J. Lenardo, A guide to cancer immunotherapy: from T cell basic science to clinical practice. Nat Rev Immunol, 2020. 20(11): p. 651-668.
Dhatchinamoorthy, K., J. D. Colbert, and K. L. Rock, Cancer Immune Evasion Through Loss of MHC Class I Antigen Presentation. Frontiers in Immunology, 2021. 12.
Jhunjhunwala, S., C. Hammer, and L. Delamarre, Antigen presentation in cancer: insights into tumour immunogenicity and immune evasion. Nat Rev Cancer, 2021. 21(5): p. 298-312.
Ennishi, D., et al., Molecular and Genetic Characterization of MHC Deficiency Identifies EZH2 as Therapeutic Target for Enhancing Immune Recognition. Cancer Discov, 2019. 9(4): p. 546-563.
Yamamoto, K., et al., Autophagy promotes immune evasion of pancreatic cancer by degrading MHC-I. Nature, 2020. 581(7806): p. 100-105.
Lee, J. H., et al., Transcriptional downregulation of MHC class I and melanoma de-differentiation in resistance to PD-1 inhibition. Nat Commun, 2020. 11(1): p. 1897.
Wei, H., et al., Suppression of autophagy by FIP200 deletion inhibits mammary tumorigenesis. Genes Dev, 2011. 25(14): p. 1510-27.
Noman, M. Z., et al., Inhibition of Vps34 reprograms cold into hot inflamed tumors and improves anti-PD-1/PD-L1 immunotherapy. Sci Adv, 2020. 6(18): p. eaax7881.
Mgrditchian, T., et al., Targeting autophagy inhibits melanoma growth by enhancing NK cells infiltration in a CCL5-dependent manner. Proc Natl Acad Sci USA, 2017. 114(44): p. E9271-E9279.
Pellegrini, P., et al., Acidic extracellular pH neutralizes the autophagy-inhibiting activity of chloroquine. Autophagy, 2014. 10(4): p. 562-571.
Karasic, T. B., et al., Effect of Gemcitabine and nab-Paclitaxel With or Without Hydroxychloroquine on Patients With Advanced Pancreatic Cancer A Phase 2 Randomized Clinical Trial. Jama Oncology, 2019. 5(7): p. 993-998.
Zeh, H. J., et al., A Randomized Phase II Preoperative Study of Autophagy Inhibition with High-Dose Hydroxychloroquine and Gemcitabine/Nab-Paclitaxel in Pancreatic Cancer Patients. Clinical Cancer Research, 2020. 26(13): p. 3126-3134.
Arnaout, A., et al., A randomized, double-blind, window of opportunity trial evaluating the effects of chloroquine in breast cancer patients. Breast Cancer Research and Treatment, 2019. 178(2): p. 327-335.
Boedtkjer, E. and S. F. Pedersen, The Acidic Tumor Microenvironment as a Driver of Cancer. Annual Review of Physiology, Vol 82, 2020. 82: p. 103-126.
Mauthe, M., et al., Chloroquine inhibits autophagic flux by decreasing autophagosome-lysosome fusion. Autophagy, 2018. 14(8): p. 1435-1455.
Schrezenmeier, E. and T. Dorner, Mechanisms of action of hydroxychloroquine and chloroquine: implications for rheumatology. Nature Reviews Rheumatology, 2020. 16(3): p. 155-166.
Qiao, Y., et al., Autophagy Inhibition by Targeting PIKfyve Potentiates Response to Immune Checkpoint Blockade in Prostate Cancer. Nat Cancer, 2021. 2: p. 978-993.
Gayle, S., et al., Identification of apilimod as a first-in-class PIKfyve kinase inhibitor for treatment of B-cell non-Hodgkin lymphoma. Blood, 2017. 129(13): p. 1768-1778.
Sharma, G., et al., A family of PIKFYVE inhibitors with therapeutic potential against autophagy-dependent cancer cells disrupt multiple events in lysosome homeostasis. Autophagy, 2019. 15(10): p. 1694-1718.
Gayle, S., et al., B-cell non-Hodgkin lymphoma: Selective vulnerability to PIKFYVE inhibition. Autophagy, 2017. 13(6): p. 1082-1083.
Harb, W. A., et al., Phase 1 Clinical Safety, Pharmacokinetics (PK), and Activity of Apilimod Dimesylate (LAM-002A), a First-in-Class Inhibitor of Phosphatidylinositol-3-Phosphate 5-Kinase (PIKfyve), in Patients with Relapsed or Refractory B-Cell Malignancies. Blood, 2017. 130.
Diefenbach, C. S. M., et al., Results of a completed phase I study of LAM-002 (apilimod dimesylate), a first-in-class phosphatidylinositol-3-phosphate 5 kinase (PIKfyve) inhibitor, administered as monotherapy or with rituximab or atezolizumab to patients with previously treated follicular lymphoma or other B-cell cancers. Journal of Clinical Oncology, 2020. 38(15).
Pili, R., et al., An open-label study to determine the maximum tolerated dose of the multitargeted tyrosine kinase inhibitor CEP-11981 in patients with advanced cancer. Invest New Drugs, 2014. 32(6): p. 1258-68.
Yoshii, S. R., et al., Systemic Analysis of Atg5-Null Mice Rescued from Neonatal Lethality by Transgenic ATG5 Expression in Neurons. Dev Cell, 2016. 39(1): p. 116-130.
Mizushima, N., et al., Dissection of autophagosome formation using Apg5-deficient mouse embryonic stem cells. J Cell Biol, 2001. 152(4): p. 657-68.
Guo, J. Y., et al., Autophagy suppresses progression of K-ras-induced lung tumors to oncocytomas and maintains lipid homeostasis. Genes Dev, 2013. 27(13): p. 1447-61.
Chen, Z., et al., seq-ImmuCC: Cell-Centric View of Tissue Transcriptome Measuring Cellular Compositions of Immune Microenvironment From Mouse RNA-Seq Data. Front Immunol, 2018. 9: p. 1286.
Petitprez, F., et al., The murine Microenvironment Cell Population counter method to estimate abundance of tissue-infiltrating immune and stromal cell populations in murine samples using gene expression. Genome Med, 2020. 12(1): p. 86.
Badrinath, S., et al., A vaccine targeting resistant tumours by dual T cell plus NK cell attack. Nature, 2022. 606(7916): p. 992-998.
van Elsas, A., A. A. Hurwitz, and J. P. Allison, Combination immunotherapy of B16 melanoma using anti-cytotoxic T lymphocyte-associated antigen 4 (CTLA-4) and granulocyte/macrophage colony-stimulating factor (GM-CSF)-producing vaccines induces rejection of subcutaneous and metastatic tumors accompanied by autoimmune depigmentation. J Exp Med, 1999. 190(3): p. 355-66.
Curran, M. A., et al., PD-1 and CTLA-4 combination blockade expands infiltrating T cells and reduces regulatory T and myeloid cells within B16 melanoma tumors. Proc Natl Acad Sci USA, 2010. 107(9): p. 4275-80.
Zhou, H., et al., Noninvasive interrogation of CD8+ T cell effector function for monitoring early tumor responses to immunotherapy. J Clin Invest, 2022. 132(16).
Sahin, U., et al., Personalized RNA mutanome vaccines mobilize poly-specific therapeutic immunity against cancer. Nature, 2017. 547(7662): p. 222-226.
Frank, M. J., et al., Autologous tumor cell vaccine induces antitumor T cell immune responses in patients with mantle cell lymphoma: A phase 1/II trial. J Exp Med, 2020. 217(9).
Bloom, M. B., et al., Identification of tyrosinase-related protein 2 as a tumor rejection antigen for the B16 melanoma. J Exp Med, 1997. 185(3): p. 453-9.
McWilliams, J. A., et al., A modified tyrosinase-related protein 2 epitope generates high-affinity tumor-specific T cells but does not mediate therapeutic efficacy in an intradermal tumor model. J Immunol, 2006. 177(1): p. 155-61.
Zaretsky, J. M., et al., Mutations Associated with Acquired Resistance to PD-1 Blockade in Melanoma. N Engl J Med, 2016. 375(9): p. 819-29.
Torrejon, D. Y., et al., Overcoming Genetically Based Resistance Mechanisms to PD-1 Blockade. Cancer Discov, 2020. 10(8): p. 1140-1157.
Gao, J., et al., Loss of IFN-gamma Pathway Genes in Tumor Cells as a Mechanism of Resistance to Anti-CTLA-4 Therapy. Cell, 2016. 167(2): p. 397-404 e9.
Ikonomov, O. C., D. Sbrissa, and A. Shisheva, Small molecule PIKfyve inhibitors as cancer therapeutics: Translational promises and limitations. Toxicol Appl Pharmacol, 2019. 383: p. 114771.
Koikawa, K., et al., Targeting Pin1 renders pancreatic cancer eradicable by synergizing with immunochemotherapy. Cell, 2021. 184(18): p. 4753-4771 e27.
Palanisamy, N., et al., The MD Anderson Prostate Cancer Patient-derived Xenograft Series (MDA PCa PDX) Captures the Molecular Landscape of Prostate Cancer and Facilitates Marker-driven Therapy Development. Clin Cancer Res, 2020. 26(18): p. 4933-4946.
Ritchie, M. E., et al., limma powers differential expression analyses for RNA-sequencing and microarray studies. Nucleic Acids Res, 2015. 43(7): p. e47.
Phipson, B., et al., Robust Hyperparameter Estimation Protects against Hypervariable Genes and Improves Power to Detect Differential Expression. Ann Appl Stat, 2016. 10(2): p. 946-963.
Robinson, M. D., D. J. McCarthy, and G. K. Smyth, edgeR: a Bioconductor package for differential expression analysis of digital gene expression data. Bioinformatics, 2010. 26(1): p. 139-40.
Subramanian, A., et al., Gene set enrichment analysis: a knowledge-based approach for interpreting genome-wide expression profiles. Proc Natl Acad Sci USA, 2005. 102(43): p. 15545-50.
Liberzon, A., et al., The Molecular Signatures Database (MSigDB) hallmark gene set collection. Cell Syst, 2015. 1(6): p. 417-425.
Yu, J., et al., Liver metastasis restrains immunotherapy efficacy via macrophage-mediated T cell elimination. Nat Med, 2021. 27(1): p. 152-164.
Robinson, D. R., et al., Integrative clinical genomics of metastatic cancer. Nature, 2017. 548(7667): p. 297-303.
Wu, Y. M., et al., Inactivation of CDK12 Delineates a Distinct Immunogenic Class of Advanced Prostate Cancer. Cell, 2018. 173(7): p. 1770-1782 e14.
Wang, W., et al., CD8(+) T cells regulate tumour ferroptosis during cancer immunotherapy. Nature, 2019. 569(7755): p. 270-274.
Hodi, F. S., et al., Immune-Modified Response Evaluation Criteria In Solid Tumors (imRECIST): Refining Guidelines to Assess the Clinical Benefit of Cancer Immunotherapy. J Clin Oncol, 2018. 36(9): p. 850-858.
Parolia, A., et al., Distinct structural classes of activating FOXA1 alterations in advanced prostate cancer. Nature, 2019. 571(7765): p. 413-418.
Cieslik, M., et al., The use of exome capture RNA-seq for highly degraded RNA with application to clinical cancer sequencing. Genome Res, 2015. 25(9): p. 1372-81.
Lanczky, A. and B. Gyorffy, Web-Based Survival Analysis Tool Tailored for Medical Research (KMplot): Development and Implementation. J Med Internet Res, 2021. 23(7): p. e27633.
Zhang, Y. & Zhang, Z. The history and advances in cancer immunotherapy: understanding the characteristics of tumor-infiltrating immune cells and their therapeutic implications. Cell. Mol. Immunol. 17, 807-821 (2020).
Litchfield, K. et al., Meta-analysis of tumor- and T cell-intrinsic mechanisms of sensitization to checkpoint inhibition. Cell 184, 596-614. e14 (2021).
Hanahan, D. Hallmarks of Cancer: New Dimensions. Cancer Discov. 12, 31-46 (2022).
Zou, W. Regulatory T cells, tumour immunity and immunotherapy. Nat. Rev. Immunol. 6, 295-307 (2006).
Peranzoni, E. et al., Macrophages impede CD8 T cells from reaching tumor cells and limit the efficacy of anti-PD-1 treatment. Proc. Natl. Acad. Sci. U.S.A 115, E4041-E4050 (2018).
Baker, A. T., Abuwarwar, M. H., Poly, L., Wilkins, S. & Fletcher, A. L. Cancer-Associated Fibroblasts and T Cells: From Mechanisms to Outcomes. J. Immunol. 206, 310-320 (2021).
Monu, N. R. & Frey, A. B. Myeloid-derived suppressor cells and anti-tumor T cells: a complex relationship. Immunol. Invest. 41, 595-613 (2012).
Wculek, S. K. et al., Dendritic cells in cancer immunology and immunotherapy. Nat. Rev. Immunol. 20, 7-24 (2020).
Domogalla, M. P., Rostan, P. V., Raker, V. K. & Steinbrink, K. Tolerance through Education: How Tolerogenic Dendritic Cells Shape Immunity. Front. Immunol. 8, 1764 (2017).
Joffre, O. P., Segura, E., Savina, A. & Amigorena, S. Cross-presentation by dendritic cells. Nat. Rev. Immunol. 12, 557-569 (2012).
Garris, C. S. et al., Successful Anti-PD-1 Cancer Immunotherapy Requires T Cell-Dendritic Cell Crosstalk Involving the Cytokines IFN-γ and IL-12. Immunity 49, 1148-1161. e7 (2018).
Mateo, J. et al., Accelerating precision medicine in metastatic prostate cancer. Nat Cancer 1, 1041-1053 (2020).
Chu, G., Shan, W., Ji, X., Wang, Y. & Niu, H. Multi-Omics Analysis of Novel Signature for Immunotherapy Response and Tumor Microenvironment Regulation Patterns in Urothelial Cancer. Front Cell Dev Biol 9, 764125 (2021).
Gerhard, G. M., Bill, R., Messemaker, M., Klein, A. M. & Pittet, M. J. Tumor-infiltrating dendritic cell states are conserved across solid human cancers. J. Exp. Med. 218, (2021).
Kepp, O. et al., Molecular determinants of immunogenic cell death elicited by anticancer chemotherapy. Cancer Metastasis Rev. 30, 61-69 (2011).
Kawano, M. et al., Dendritic cells combined with doxorubicin induces immunogenic cell death and exhibits antitumor effects for osteosarcoma. Oncol. Lett. 11, 2169-2175 (2016).
Shurin, G. V., Tourkova, I. L., Kaneno, R. & Shurin, M. R. Chemotherapeutic agents in noncytotoxic concentrations increase antigen presentation by dendritic cells via an IL-12-dependent mechanism. J. Immunol. 183, 137-144 (2009).
Liang, Y.-H. et al., Chemotherapy agents stimulate dendritic cells against human colon cancer cells through upregulation of the transporter associated with antigen processing. Sci. Rep. 11, 9080 (2021).
Michels, T. et al., Paclitaxel promotes differentiation of myeloid-derived suppressor cells into dendritic cells in vitro in a TLR4-independent manner. J. Immunotoxicol. 9, 292-300 (2012).
Zhou, X. et al., Chemotherapy combined with dendritic cell vaccine and cytokine-induced killer cells in the treatment of colorectal carcinoma: a meta-analysis. Cancer Manag. Res. 10, 5363-5372 (2018).
Yu, B. et al., Effective combination of chemotherapy and dendritic cell administration for the treatment of advanced-stage experimental breast cancer. Clin. Cancer Res. 9, 285-294 (2003).
Rob, L. et al., Safety and efficacy of dendritic cell-based immunotherapy DCVAC/OvCa added to first-line chemotherapy (carboplatin plus paclitaxel) for epithelial ovarian cancer: a phase 2, open-label, multicenter, randomized trial. J Immunother Cancer 10, (2022).
Cohen, P., Cross, D. & Jänne, P. A. Kinase drug discovery 20 years after imatinib: progress and future directions. Nat. Rev. Drug Discov. 20, 551-569 (2021).
Arora, A. & Scholar, E. M. Role of tyrosine kinase inhibitors in cancer therapy. J. Pharmacol. Exp. Ther. 315, 971-979 (2005).
Kannaiyan, R. & Mahadevan, D. A comprehensive review of protein kinase inhibitors for cancer therapy. Expert Rev. Anticancer Ther. 18, 1249-1270 (2018).
Madhusudan, S. & Ganesan, T. S. Tyrosine kinase inhibitors in cancer therapy. Clin. Biochem. 37, 618-635 (2004).
Wang, H. et al., Tumor immunological phenotype signature-based high-throughput screening for the discovery of combination immunotherapy compounds. Sci Adv 7, (2021).
Moynihan, K. D. et al., Eradication of large established tumors in mice by combination immunotherapy that engages innate and adaptive immune responses. Nat. Med. 22, 1402-1410 (2016).
Hu-Lieskovan, S. et al., Improved antitumor activity of immunotherapy with BRAF and MEK inhibitors in BRAF(V600E) melanoma. Sci. Transl. Med. 7, 279ra41 (2015).
Baumann, D. et al., Proimmunogenic impact of MEK inhibition synergizes with agonist anti-CD40 immunostimulatory antibodies in tumor therapy. Nat. Commun. 11, 2176 (2020).
Peng, D. H. et al., Th17 cells contribute to combination MEK inhibitor and anti-PD-L1 therapy resistance in KRAS/p53 mutant lung cancers. Nat. Commun. 12, 2606 (2021).
Gong, K. et al., EGFR inhibition triggers an adaptive response by co-opting antiviral signaling pathways in lung cancer. Nat Cancer 1, 394-409 (2020).
Ribas, A. et al., PD-L1 blockade in combination with inhibition of MAPK oncogenic signaling in patients with advanced melanoma. Nat. Commun. 11, 6262 (2020).
Corsello, S. M. et al., The Drug Repurposing Hub: a next-generation drug library and information resource. Nat. Med. 23, 405-408 (2017). Martin, J. K., 2nd et al., A Dual-Mechanism Antibiotic Kills Gram-Negative Bacteria and Avoids Drug Resistance. Cell 181, 1518-1532. e14 (2020).
Tanaka, K. et al., Targeting Aurora B kinase prevents and overcomes resistance to EGFR inhibitors in lung cancer by enhancing BIM- and PUMA-mediated apoptosis. Cancer Cell 39, 1245-1261. e6 (2021).
Eisenhauer, E. A. et al., New response evaluation criteria in solid tumours: revised RECIST guideline (version 1.1). Eur. J. Cancer 45, 228-247 (2009).
Riaz, N. et al., Tumor and Microenvironment Evolution during Immunotherapy with Nivolumab. Cell 171, 934-949. e16 (2017).
Krishna, S. et al., PIKfyve Regulates Vacuole Maturation and Nutrient Recovery following Engulfment. Dev. Cell 38, 536-547 (2016).
Vicinanza, M. et al., PI(5)P regulates autophagosome biogenesis. Mol. Cell 57, 219-234 (2015).
Giridharan, S. S. P. et al., Lipid kinases VPS34 and PIKfyve coordinate a phosphoinositide cascade to regulate retriever-mediated recycling on endosomes. Elife 11, (2022).
Qian, J. et al., A pan-cancer blueprint of the heterogeneous tumor microenvironment revealed by single-cell profiling. Cell Res. 30, 745-762 (2020).
Sade-Feldman, M. et al., Defining T Cell States Associated with Response to Checkpoint Immunotherapy in Melanoma. Cell 175, 998-1013. e20 (2018).
Chow, R. D. et al., Distinct Mechanisms of Mismatch-Repair Deficiency Delineate Two Modes of Response to Anti-PD-1 Immunotherapy in Endometrial Carcinoma. Cancer Discov. 13, 312-331 (2023).
Choy, C. H. et al., Lysosome enlargement during inhibition of the lipid kinase PIKfyve proceeds through lysosome coalescence. J. Cell Sci. 131, (2018).
Hessvik, N. P. et al., PIKfyve inhibition increases exosome release and induces secretory autophagy. Cell. Mol. Life Sci. 73, 4717-4737 (2016).
Baranov, M. V. et al., The Phosphoinositide Kinase PIKfyve Promotes Cathepsin-S-Mediated Major Histocompatibility Complex Class II Antigen Presentation. iScience 11, 160-177 (2019).
Cai, X. et al., PIKfyve, a class III PI kinase, is the target of the small molecular IL-12/IL-23 inhibitor apilimod and a player in Toll-like receptor signaling. Chem. Biol. 20, 912-921 (2013).
Wada, Y. et al., Apilimod inhibits the production of IL-12 and IL-23 and reduces dendritic cell infiltration in psoriasis. PLoS One 7, e35069 (2012).
Krausz, S. et al., Brief report: a phase IIa, randomized, double-blind, placebo-controlled trial of apilimod mesylate, an interleukin-12/interleukin-23 inhibitor, in patients with rheumatoid arthritis. Arthritis Rheum. 64, 1750-1755 (2012).
Kang, Y.-L. et al., Inhibition of PIKfyve kinase prevents infection by Zaire ebolavirus and SARS-CoV-2. Proc. Natl. Acad. Sci. U.S.A 117, 20803-20813 (2020).
Hildner, K. et al., Batf3 deficiency reveals a critical role for CD8alpha+ dendritic cells in cytotoxic T cell immunity. Science 322, 1097-1100 (2008).
Durai, V. et al., Cryptic activation of an Irf8 enhancer governs cDC1 fate specification. Nat. Immunol. 20, 1161-1173 (2019).
Ferris, S. T. et al., cDC1 prime and are licensed by CD4+ T cells to induce anti-tumour immunity. Nature 584, 624-629 (2020).
Hogquist, K. A. et al., T cell receptor antagonist peptides induce positive selection. Cell 76, 17-27 (1994).
Lin, H. et al., Stanniocalcin 1 is a phagocytosis checkpoint driving tumor immune resistance. Cancer Cell 39, 480-493. e6 (2021).
Ouaaz, F., Arron, J., Zheng, Y., Choi, Y. & Beg, A. A. Dendritic cell development and survival require distinct NF-kappaB subunits. Immunity 16, 257-270 (2002).
Ghislat, G. et al., NF-κB-dependent IRF1 activation programs cDC1 dendritic cells to drive antitumor immunity. Sci Immunol 6, (2021).
Wang, J. et al., Distinct roles of different NF-kappa B subunits in regulating inflammatory and T cell stimulatory gene expression in dendritic cells. J. Immunol. 178, 6777-6788 (2007).
Baratin, M. et al., Homeostatic NF-κB Signaling in Steady-State Migratory Dendritic Cells Regulates Immune Homeostasis and Tolerance. Immunity 42, 627-639 (2015).
Zhang, Q., Lenardo, M. J. & Baltimore, D. 30 Years of NF-κB: A Blossoming of Relevance to Human Pathobiology. Cell 168, 37-57 (2017).
Yum, S., Li, M., Fang, Y. & Chen, Z. J. TBK1 recruitment to STING activates both IRF3 and NF-κB that mediate immune defense against tumors and viral infections. Proc. Natl. Acad. Sci. U.S.A 118, (2021).
Zhou, B. et al., Extracellular SQSTM1 mediates bacterial septic death in mice through insulin receptor signalling. Nat Microbiol 5, 1576-1587 (2020).
Aflaki, E. et al., Lysosomal storage and impaired autophagy lead to inflammasome activation in Gaucher macrophages. Aging Cell 15, 77-88 (2016).
Schlutermann, D. et al., FIP200 controls the TBK1 activation threshold at SQSTM1/p62-positive condensates. Sci. Rep. 11, 13863 (2021).
Matsumoto, G., Shimogori, T., Hattori, N. & Nukina, N. TBK1 controls autophagosomal engulfment of polyubiquitinated mitochondria through p62/SQSTM1 phosphorylation. Hum. Mol. Genet. 24, 4429-4442 (2015).
Prabakaran, T. et al., Attenuation of cGAS-STING signaling is mediated by a p62/SQSTM1-dependent autophagy pathway activated by TBK1. EMBO J. 37, (2018).
Zhong, Z. et al., NF-κB Restricts Inflammasome Activation via Elimination of Damaged Mitochondria. Cell 164, 896-910 (2016).
Lobb, I. T. et al., A Role for the Autophagic Receptor, SQSTM1/p62, in Trafficking NF-κB/RelA to Nucleolar Aggresomes. Mol. Cancer Res. 19, 274-287 (2021).
Shi, J. et al., Cleavage of sequestosome 1/p62 by an enteroviral protease results in disrupted selective autophagy and impaired NFKB signaling. Autophagy 9, 1591-1603 (2013).
Schwob, A. et al., SQSTM-1/p62 potentiates HTLV-1 Tax-mediated NF-κB activation through its ubiquitin binding function. Sci. Rep. 9, 16014 (2019).
Murakami, K. et al., A RUNX-CBFβ-driven enhancer directs the Irf8 dose-dependent lineage choice between DCs and monocytes. Nat. Immunol. 22, 301-311(2021).
Kim, S. M. et al., Targeting cancer metabolism by simultaneously disrupting parallel nutrient access pathways. J. Clin. Invest. 126, 4088-4102 (2016).
Hou, J.-Z. et al., Inhibition of PIKfyve using YM201636 suppresses the growth of liver cancer via the induction of autophagy. Oncol. Rep. 41, 1971-1979 (2019).
O'Connell, C. E. & Vassilev, A. Combined Inhibition of p38MAPK and PIKfyve Synergistically Disrupts Autophagy to Selectively Target Cancer Cells. Cancer Res. 81, 2903-2917 (2021).
Martins, K. A. O., Bavari, S. & Salazar, A. M. Vaccine adjuvant uses of poly-IC and derivatives. Expert Rev. Vaccines 14, 447-459 (2015).
Zhong, L. et al., Small molecules in targeted cancer therapy: advances, challenges, and future perspectives. Signal Transduct Target Ther 6, 201 (2021).
Shen, C.-I. et al., Comparison of the outcome between immunotherapy alone or in combination with chemotherapy in EGFR-mutant non-small cell lung cancer. Sci. Rep. 11, 16122 (2021).
O'Shea, P. J. et al., Outcomes of immunotherapy (ICI) alone vs tyrosine kinase inhibitors (TKI) alone versus ICI and TKI combined in renal cell carcinoma brain metastasis. J. Clin. Orthod. 39, 2030-2030 (2021).
Sugiyama, E. et al., Blockade of EGFR improves responsiveness to PD-1 blockade in EGFR-mutated non-small cell lung cancer. Sci Immunol 5, (2020).
Zolov, S. N. et al., In vivo, Pikfyve generates PI(3,5)P₂, which serves as both a signaling lipid and the major precursor for PI5P. Proc. Natl. Acad. Sci. U.S.A 109, 17472-17477 (2012).
Xiao, Y. et al., The kinase TBK1 functions in dendritic cells to regulate T cell homeostasis, autoimmunity, and antitumor immunity. J. Exp. Med. 214, 1493-1507 (2017).
Fitzgerald, K. A. et al., IKKepsilon and TBK1 are essential components of the IRF3 signaling pathway. Nat. Immunol. 4, 491-496 (2003).
Xia, Y., Shen, S. & Verma, I. M. NF-κB, an active player in human cancers. Cancer Immunol Res 2, 823-830 (2014).
Romero, P. et al., The Human Vaccines Project: A roadmap for cancer vaccine development. Sci. Transl. Med. 8, 334ps9 (2016).
Steinman, R. M. & Pope, M. Exploiting dendritic cells to improve vaccine efficacy. J. Clin. Invest. 109, 1519-1526 (2002).
Melief, C. J. M. et al., Strong vaccine responses during chemotherapy are associated with prolonged cancer survival. Sci. Transl. Med. 12, (2020).
Tanyi, J. L. et al., Personalized cancer vaccine effectively mobilizes antitumor T cell immunity in ovarian cancer. Sci. Transl. Med. 10, (2018).
Carreno, B. M. et al., Cancer immunotherapy. A dendritic cell vaccine increases the breadth and diversity of melanoma neoantigen-specific T cells. Science 348, 803-808 (2015).
Rosenblatt, J. et al., Individualized vaccination of AML patients in remission is associated with induction of antileukemia immunity and prolonged remissions. Sci. Transl. Med. 8, 368ra171 (2016).
Daud, A. I. Negative but not futile: MAGE-A3 immunotherapeutic for melanoma. The lancet oncology vol. 19 852-853 (2018).
Huang, P.-T., Einav, S. & Asquith, C. R. M. PIKfyve: a lipid kinase target for COVID-19, cancer and neurodegenerative disorders. Nat. Rev. Drug Discov. 20, 730 (2021).
Mead, B. E. et al., Screening for modulators of the cellular composition of gut epithelia via organoid models of intestinal stem cell differentiation. Nat Biomed Eng 6, 476-494 (2022).
Tanikawa, T. et al., Interleukin-10 ablation promotes tumor development, growth, and metastasis. Cancer Res. 72, 420-429 (2012).
Lin, H. et al., Host expression of PD-L1 determines efficacy of PD-L1 pathway blockade-mediated tumor regression. J. Clin. Invest. 128, 805-815 (2018).
Korotkevich, G. et al., Fast gene set enrichment analysis. bioRxiv 060012 (2021) doi:10.1101/060012.
Hao, Y. et al., Integrated analysis of multimodal single-cell data. Cell 184, 3573-3587. e29 (2021).

Having now fully described the compounds, compositions, uses, and methods herein, it will be understood by those of skill in the art that the same can be performed within a wide and equivalent range of conditions, formulations, and other parameters without affecting the scope of the compounds, compositions, uses, and methods provided herein, or any embodiment thereof.

All patents, patent applications, and publications cited herein are fully incorporated by reference herein in their entirety.

1. A method of treating cancer in a subject in need thereof, the method comprising administering a therapeutically effective amount of:
(a) 13-isobutyl-4-methyl-10-(pyrimidin-2-ylamino)-1,2,4,7,8,13-hexahydro-6H-indazolo[5,4-a]pyrrolo[3,4-c]carbazol-6-one (ESK981); or
(b) N-[(E)-(3-methylphenyl)methylideneamino]-6-morpholin-4-yl-2-(2-pyridin-2-ylethoxy)pyrimidin-4-amine (apilimod),
to the subject in combination with a therapeutically effective amount of an immunotherapy, wherein the immunotherapy is adoptive cell therapy (ACT) or a personalized cancer vaccine.

2. The method of claim 1, wherein the immunotherapy is CD8⁺ T cell-dependent immunotherapy.

3. The method of claim 1, wherein the immunotherapy is major histocompatibility complex (MHC) class I-dependent immunotherapy.

4. The method of claim 1, wherein the immunotherapy is adoptive cell therapy.

5. The method of claim 4, wherein the adoptive cell therapy comprises T cells, dendritic cells, macrophages, peripherial blood mononuclear cells (PBMCs), or a combination thereof.

6. The method of claim 5, wherein the adoptive cell therapy comprises T cells.

7. The method of claim 6, wherein the adoptive cell therapy comprises engineered cells expressing a heterologous T cell receptor, a chimeric antigen receptor, or a combination thereof.

8. The method of claim 7, wherein the adoptive cell therapy comprises engineered cells expressing a heterologous T cell receptor.

9. The method of claim 8, wherein the heterologous T cell receptor comprises an extracellular binding moiety that specifically binds a tumor-associated antigen.

10. The method of claim 8, wherein the heterologous T cell receptor comprises an anti-CD3 binding moiety.

11. The method of claim 8, wherein the heterologous T cell receptor comprises an anti-CD28 binding moiety.

12-16. (canceled)

17. The method of claim 1, wherein the immunotherapy is a personalized cancer vaccine.

18. The method of claim 17, wherein the personalized cancer vaccine comprises a phagocytosis stimulating agent, an immunostimulatory adjuvant, and attenuated cancer cells.

19. The method of claim 18, wherein the attenuated cancer cells are obtained from a tumor of the subject.

20. The method of claim 18, wherein the immunostimulatory adjuvant comprises a Toll like receptor (TLR) agonist.

21. The method of claim 19, wherein the immunostimulatory adjuvant comprises an anti-CD3 antibody.

22. The method of claim 19, wherein the immunostimulatory adjuvant comprises an anti-CD28 antibody.

23. A method of treating cancer in a subject in need thereof, the method comprising administering a therapeutically effective amount of ESK981 or apilimod to the subject in combination with a therapeutically effective amount of an immunotherapy, wherein the cancer is characterized as overexpressing PIKfyve.

24-27. (canceled)

28. The method of claim 1, wherein the cancer is adrenal cancer, acinic cell carcinoma, acoustic neuroma, acral lentigious melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatocellular carcinoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogeous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, preimary peritoneal cancer, prostate cancer, pancreatic cancer, pharyngeal cancer, pseudomyxoma periotonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, or Wilms' tumor.

29. (canceled)

30. The method of claim 1, wherein a therapeutically effective amount of ESK981 is administered to the subject.

31. (canceled)
